(19) United States
(12) Patent Application Publication
(10) Pub. No.: US 2003/0092646 A1
(43) Pub. Date: May 15, 2003

(54) Method and reagent for the inhibition of CD20

(76) Inventors: Lawrence Blatt, Boulder, CO (US); James McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/780,164

(22) Filed: Feb. 9, 2001

Related U.S. Application Data
(60) Provisional application No. 60/185,516, filed on Feb. 28, 2000.

(51) Int. Cl. A61K 48/00; C07H 21/02; C07H 21/04

(52) U.S. Cl. 514/44; 536/23.1

(57) ABSTRACT

The present invention relates to nucleic acid molecules, including antisense and enzymatic nucleic acid molecules, such as hammerhead ribozymes, DNAzymes, and antisense, which modulate the expression of the CD20 gene.

### BACKGROUND OF THE INVENTION

This invention claims priority from Blatt, U.S. S. No. (60/185,516), filed Feb. 28, 2000, entitled “METHOD AND REAGENT FOR THE INHIBITION OF CD20”. This application is hereby incorporated by reference herein in its entirety including the drawings.

The present invention concerns compounds, compositions, and methods for the study, diagnosis, and treatment of conditions and diseases that respond to the modulation of CD20 antigen. Specifically, the instant invention provides for compositions and methods for the treatment of diseases associated with the level of CD20.

The following is a brief description of the current understanding of CD20, its biological function, and therapeutic relevance. The discussion is not meant to be complete and is provided only for understanding the invention that follows. The summary is not an admission that any of the work described below is prior art to the claimed invention.

The vertebrate immune system has evolved to include a number of organs and cell types which specifically recognize foreign antigens (e.g., antibody generators) from invading pathogens. The immune response, which is mediated by lymphocytes, seeks out and destroys the invading foreign bodies through specific recognition of antibodies and subsequent destruction of foreign bodies. Lymphocytes, which represent about 30% of the total number of white blood cells in the adult human circulatory system, are produced in the primary lymphoid organs, the thymus, spleen, and bone marrow. The two major sub-types of lymphocytes are B-cells and T-cells.

T-cells, which develop in the thymus, are responsible for cell-mediated immunity. B-cells, which develop in the adult bone marrow (or fetal liver), produce antibodies and are responsible for humoral immunity. T-cells are activated by the binding of major histocompatability complex (MHC) glycoproteins on the surface of an antigenic cell to T-cell receptors. Activated T-cells release regulatory molecules, such as interleukins, that can stimulate B-cell differentiation. Activated B-cells develop into antibody secreting cells which are filled with an extensive rough endoplasmic reticulum for the production of immunoglobulins against an antigen. B-cell diversity is central to the effective functioning to the immune system. An activated B-cell can produce large quantities of antibody in response to a given antigen. Normally, this antibody production is modulated in response to the neutralization of the antigen. However, when the production of B-cells is dysregulated, such proliferation can result in B-cell lymphoma.

CD20 is a 35 kDa cell surface phosphoprotein expressed exclusively in mature B lymphocytes (Rosenthal et al., 1983, J. Immunol., 131, 232-237; Stashenko et al., 1980, J. Immunol. 125, 1678-1685). This B-cell lineage specific antigen is found on all tumor cells within most B-cell lymphomas. The increased expression of CD20 appears to be associated with tumor cell proliferation, although the magnitude of expression varies among different types of lymphoid tumors. CD20 is a transmembrane protein with four transmembrane domains with both C- and N-terminals located in the cytoplasm. The primary structure of CD20 has been determined by molecular cloning (Einfeld et al., 1986, EMBO J., 7, 711-717; Tedder et al., 1988, PNAS USA, 85, 208-212) and resembles those of ion channel and ion transporter proteins. When expressed in fibroblasts, CD20 functions as a calcium-permeable cation channel which is activated by the insulin-like growth factor-I (IGF-I) receptor (Kanzaki et al., 1997, J. Biol. Chem., 272, 4964-69). Modulation of cell growth is observed in fibroblasts expressing CD20. In CD20 expressing Balb/c 3T3 fibroblasts, CD20 expression accelerates cell cycle progression through the G, phase and enables cells to enter S phase in cell culture medium containing low extracellular calcium (Kanzaki et al., 1995, J. Biol. Chem., 270, 13099-04). In B-lymphocytes, CD20 appears to function directly in the regulation of transmembrane Ca²⁺ conductance (Bubien et al., 1993, J. Cell. Biol., 121, 1121-1132). In lymphocytes, CD20 has been shown to be associated with src family tyrosine kinases, and is phosphorylated by protein kinases such as calmodulin-dependant protein kinase. Monoclonal antibody (mAB) binding to CD20 alters cell cycle progression and differentiation in B-lymphocytes, thus indicating that CD20 plays an essential role in B-cell function (for a review of CD20 function, see Tedder and Engel, 1994, Immunol. Today, 15(9), 450-4).

As such, CD20 has the potential for providing a molecular target for the treatment of diseases such as B-cell lymphomas. The use of monoclonal antibodies targeting CD20 has been extensively described (for a review, see Weiner, 1999, Semin. Oncol., 26, 43-51; Gopal and Press, 1999, J. Lab. Clin. Med., 134, 445-450; White et al., 1999, Pharm. Sci. Technol. Today, 2, 95-101). Rituxan™ is an chimeric anti-CD20 monoclonal antibody which has been used widely both as a single agent and together with chemotherapy in patients with newly diagnosed and relapsed lymphomas (Davis et al, 1999, J. Clin. Oncol., 17, 1851-1857; Solal-Celigny et al., 1999, Blood, 94, abstract 2802; Foran et al., 2000, J. Clin. Oncol., 18, 317-324). In addition, the use of radiolabeled antibody conjugates has been described. Bexxar™ is an I-131 conjugated antibody which is believed to work through a dual mechanism of action resulting from the immune system activity of the mAB and the therapeutic effects of the iodine (1-131) radioisotope. The use of Bexxar in patients with transformed low-grade lymphoma is described by Zelenetz et al., 1999, Blood, 94, abstract 2806. Zevalin™ is an anti-CD20 murine IgG1 kappa monoclonal antibody, conjugated to tiuxetan, which can be conjugated with either In-111 for imaging/dosimetry or yttrium-90 for therapeutic use. A controlled study of Zevalin compared to Rituxan for patients with B-cell lymphoma is reported by Witzig et al., 1999, Blood, 94, abstract 2805.

Although the use of monoclonal antibodies and conjugates has provided therapeutic value in the treatment of lymphomas, their efficacy and safety are by no means ideal. The use of monoclonal antibodies can be limiting due to factors including but not limited to toxicity, immunogenicity, and tumor resistance. In addition, radioisotope conjugated mABs can potentially damage non-pathogenic tissues, resulting in malignancy outside the scope of the original pathology. The route of administration of many of these compounds is intravenous infusion. Infusion related side effects can be problematic. Winkler et al., 1999, Blood, 94(7), 2217-2224, describe Cytokine-release syndrome and poor overall efficacy in patients with B-cell chronic lymphocytic leukemia and high lymphocyte counts after treatment with an anti-CD20 monoclonal antibody (rituximab). As such, there exists a need for safe and effective therapeutics in order to replace or compliment existing lymphoma treatment strategies.

### SUMMARY OF THE INVENTION

The invention features novel nucleic acid-based techniques [e.g., enzymatic nucleic acid molecules (ribozymes), antisense nucleic acids, 2-5A antisense chimeras, triplex DNA, antisense nucleic acids containing RNA cleaving chemical groups] and methods for their use to modulate the expression of CD20.

The description below of the various aspects and embodiments is provided with reference to the exemplary gene CD20. However, the various aspects and embodiments are also directed to other genes, including those which express CD20-like proteins involved in B-cell proliferation. Those additional genes can be analyzed for target sites using the methods described for CD20. Thus, the inhibition and the effects of such inhibition of the other genes can be performed as described herein.

In a preferred embodiment, the invention features the use of one or more of the nucleic acid-based techniques independently or in combination to inhibit the expression of the genes encoding CD20. Specifically, the invention features the use of nucleic acid-based techniques to specifically inhibit the expression of CD20 gene (an exemplary CD20 sequence is found at GenBank Accession No. X07203).

In another preferred embodiment, the invention features the use of an enzymatic nucleic acid molecule, preferably in the hammerhead, NCH (Inozyme), G-cleaver, amberzyme, zinzyme and/or DNAzyme motif, to inhibit the expression of CD20 gene.

By “inhibit” it is meant that the activity of CD20 or level of RNAs or equivalent RNAs encoding one or more protein subunits of CD20 is reduced below that observed in the absence of the nucleic acid molecules of the invention. In one embodiment, inhibition with enzymatic nucleic acid molecule preferably is below that level observed in the presence of an enzymatically inactive or attenuated molecule that is able to bind to the same site on the target RNA, but is unable to cleave that RNA. In another embodiment, inhibition with antisense oligonucleotides is preferably below that level observed in the presence of, for example, an oligonucleotide with scrambled sequence or with mismatches. In another embodiment, inhibition of CD20 genes with the nucleic acid molecule of the instant invention is greater than in the presence of the nucleic acid molecule than in its absence.

By “enzymatic nucleic acid molecule” it is meant a nucleic acid molecule which has complementarity in a substrate-binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% may also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

By “nucleic acid molecule” as used herein is meant a molecule having nucleotides. The nucleic acid can be single, double, or multiple stranded and may comprise modified or unmodified nucleotides or non-nucleotides or various mixtures and combinations thereof.

By “enzymatic portion” or “catalytic domain” is meant that portion/region of the enzymatic nucleic acid molecule essential for cleavage of a nucleic acid substrate (for example, see FIGS. 1-5).

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-5. That is, these arms contain sequences within a enzymatic nucleic acid which are intended to bring enzymatic nucleic acid and target RNA together through complementary base-pairing interactions. The enzymatic nucleic acid of the invention can have binding arms that are contiguous or non-contiguous and may be of varying lengths. The length of the binding arm(s) are preferably greater than or equal to four nucleotides and of sufficient length to stably interact with the target RNA; preferably 12-100 nucleotides; more preferably 14-24 nucleotides long (see for example Werner and Uhlenbeck, supra; Hamman et al., supra; Hampel et al., EP0360257; Berzal-Herrance et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “Inozyme” or “NCH” motif is meant, an enzymatic nucleic acid molecule comprising a motif as is generally described as NCH Rz in FIG. 2. Inozymes possess endonuclease activity to cleave RNA substrates having a cleavage triplet NCH/, where N is a nucleotide, C is cytidine and H is adenosine, uridine or cytidine, and/represents the cleavage site. H is used interchangeably with X. Inozymes can also possess endonuclease activity to cleave RNA substrates having a cleavage triplet NCH/, where N is a nucleotide, C is cytidine, and / represents the cleavage site. “I” in FIG. 2 represents an Inosine nucleotide, preferably a ribo-Inosine or xylo-Inosine nucleoside.

By “G-cleaver” motif is meant, an enzymatic nucleic acid molecule comprising a motif as is generally described as G-cleaver in FIG. 2. G-cleavers possess endonuclease activity to cleave RNA substrates having a cleavage triplet NYN/, where N is a nucleotide, Y is uridine or cytidine and / represents the cleavage site. G-cleavers may be chemically modified as is generally shown in FIG. 2.

By “amberzyme” motif is meant, an enzymatic nucleic acid molecule comprising a motif as is generally described in FIG. 3. Amberzymes possess endonuclease activity to cleave RNA substrates having a cleavage triplet NG/N, where N is a nucleotide, G is guanosine, and / represents the cleavage site. Amberzymes can be chemically modified to increase nuclease stability through substitutions as are generally shown in FIG. 3. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

By “zinzyme” motif is meant, an enzymatic nucleic acid molecule comprising a motif as is generally described in FIG. 4. Zinzymes possess endonuclease activity to cleave RNA substrates having a cleavage triplet including but not limited to YG/Y, where Y is uridine or cytidine, and G is guanosine and/represents the cleavage site. Zinzymes may be chemically modified to increase nuclease stability through substitutions as are generally shown in FIG. 4, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group for its activity, In particular embodiments the enzymatic nucleic acid molecule can have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 5 and is generally reviewed in Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. Additional DNAzyme motifs can be selected for using techniques similar to those described in these references, and hence, are within the scope of the present invention.

By “sufficient length” is meant an oligonucleotide of greater than or equal to 3 nucleotides that is of a length great enough to provide the intended function under the expected condition.

For example, for binding arms of enzymatic nucleic acid “sufficient length” means that the binding arm sequence is long enough to provide stable binding to a target site under the expected binding conditions. Preferably, the binding arms are not so long as to prevent useful turnover.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “equivalent” RNA to CD20 is meant to include those naturally occurring RNA molecules having homology (partial or complete) to CD20 proteins or encoding for proteins with similar function as CD20 in various organisms, including but not limited to parasites, human, rodent, primate, rabbit, and pig. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule may be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNAse H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense chimera” is meant an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

By “triplex forming oligonucleotides” is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

By “gene” it is meant a nucleic acid that encodes an RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2position of a β-D-ribo-furanose moiety.

By “decoy RNA” is meant a RNA molecule that mimics the natural binding domain for a ligand. The decoy RNA therefore competes with natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art.

Several varieties of naturally occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecules that cleave the specified sites in CD20-specific RNAs represent a novel therapeutic approach to treat a variety of pathologic indications, including but not limited to lymphoma, leukemia, and inflammatory arthropathy. Specifically, the enzymatic nucleic acid molecules of the instant invention can be used to treat lymphoma, leukemia, and arthropathy, including but not limited to B-cell lymphoma, low-grade or follicular non-Hodgkin's lymphoma (NHL), bulky low-grade or follicular NUL, lypmphocytic leukemia, HIV associated NHL, mantle-cell lymphoma (MCL), immunocytoma (IMC), small B-cell lymphocytic lymphoma, immune thrombocytopenia, and inflammatory arthropathy.

In one of the preferred embodiments of the inventions described herein, the enzymatic nucleic acid molecule is formed in a hammerhead or hairpin motif, but may also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183. Examples of hairpin motifs are described by Hampel et al., EP0360257, Hampel and Tritz, 1989 Biochemistry 28, 4929, Feldstein et al., 1989, Gene 82, 53, Haseloff and Gerlach, 1989, Gene, 82, 43, Hampel et al., 1990 Nuicleic Acids Res. 18, 299; and Chowrira & McSwiggen, U.S. Pat. No. 5,631,359. The hepatitis delta virus motif is described by Perrotta and Been, 1992 Biochemistry 31, 16. The RNase P motif is described by Guerrier-Takada et al., 1983 Cell 35, 849; Forster and Altman, 1990, Science 249, 783; and Li and Altman, 1996, Nucleic Acids Res. 24, 835. The Neurospora VS RNA ribozyme motif is described by Collins (Saville and Collins, 1990 Cell 61, 685-696; Saville and Collins, 1991 Proc Natl. Acad. Sci. USA 88, 8826-8830; Collins and Olive, 1993 Biochemistry 32, 2795-2799; and Guo and Collins, 1995, EMBO. J. 14, 363). Group II introns are described by Griffin et al., 1995, Chem. Biol. 2, 761; Michels and Pyle, 1995, Biochemistry 34, 2965; and Pyle et al., International PCT Publication No. WO 96/22689. The Group I intron is described by Cech et al., U.S. Pat. No. 4,987,071. DNAzymes are described by Usman et al., International PCT Publication No. WO 95/11304; Chartrand et al., 1995, NAR 23, 4092; Breaker et al., 1995, Chem. Bio. 2, 655; and Santoro et al., 1997, PNAS 94, 4262. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs include the Aptazyme (Breaker et al., WO 98/43993), Amberzyme (Class I motif; FIG. 3; Beigelman et al., International PCT publication No. WO 99/55857) and Zinzyme (Beigelman et al., International PCT publication No. WO 99/55857), all these references are incorporated by reference herein in their totalities, including drawings and can also be used in the present invention. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In preferred embodiments of the present invention, a nucleic acid molecule of the instant invention can be between 13 and 100 nucleotides in length. Exemplary enzymatic nucleic acid molecules of the invention are shown in Tables III-XIII. For example, enzymatic nucleic acid molecules of the invention are preferably between 15 and 50 nucleotides in length, more preferably between 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between 15 and 40 nucleotides in length, more preferably between 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between 15 and 75 nucleotides in length, more preferably between 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between 10 and 40 nucleotides in length, more preferably between 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for the nucleic acid molecule are of length and conformation sufficient and suitable for the nucleic acid molecule to catalyze a reaction contemplated herein. The length of the nucleic acid molecules of the instant invention are not limiting within the general limits stated.

Preferably, a nucleic acid molecule that down regulates the replication of CD20 comprises between 12 and 100 bases complementary to a RNA molecule of CD20. Even more preferably, a nucleic acid molecule that down regulates the replication of CD20 comprises between 14 and 24 bases complementary to a RNA molecule of CD20.

In a preferred embodiment, the invention provides a method for producing a class of nucleic acid-based gene inhibiting agents which exhibit a high degree of specificity for the RNA of a desired target. For example, the enzymatic nucleic acid molecule is preferably targeted to a highly conserved sequence region of target RNAs encoding CD20 proteins such that specific treatment of a disease or condition can be provided with either one or several nucleic acid molecules of the invention. Such nucleic acid molecules can be delivered exogenously to specific tissues or cellular targets as required. Alternatively, the nucleic acid molecules (e.g., ribozymes and antisense) can be expressed from DNA and/or RNA vectors that are delivered to specific cells.

In a preferred embodiment, the invention features the use of nucleic acid-based inhibitors of the invention to specifically target genes that share homology with the CD20 gene.

As used in herein “cell” is used in its usual biological sense, and does not refer to an entire multicellular organism, e.g., specifically does not refer to a human. The cell may be present in an organism which may be a human but is preferably a non-human multicellular organism, e.g., birds, plants and mammals such as cows, sheep, apes, monkeys, swine, dogs, and cats. The cell may be prokaryotic (e.g., bacterial cell) or eukaryotic (e.g., mammalian or plant cell).

By “CD20 proteins” is meant, a protein or a mutant protein derivative thereof, comprising a cell surface phosphoprotein which is expressed, for example, in mature B lymphocytes.

By “highly conserved sequence region” is meant, a nucleotide sequence of one or more regions in a target gene does not vary significantly from one generation to the other or from one biological system to the other.

The nucleic acid-based inhibitors of CD20 expression are useful for the prevention and/or treatment of diseases and conditions such as lymphoma, leukemia, and arthropathy, including but not limited to B-cell lymphoma, low-grade or follicular non-Hodgkin's lymphoma (NHL), bulky low-grade or follicular NHL, lypmphocytic leukemia, HIV associated NHL, mantle-cell lymphoma (MCL), immunocytoma (IMC), small B-cell lymphocytic lymphoma, immune thrombocytopenia, inflammatory arthropathy, and any other diseases or conditions that are related to or will respond to the levels of CD20 in a cell or tissue, alone or in combination with other therapies.

By “related” is meant that the reduction of CD20 expression (specifically CD20 gene) RNA levels and thus reduction in the level of the respective protein will relieve, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In preferred embodiments, the enzymatic nucleic acid inhibitors comprise sequences, which are complementary to the substrate sequences in Tables III to VIII. Examples of such enzymatic nucleic acid molecules also are shown in Tables III to VIII. Examples of such enzymatic nucleic acid molecules consist essentially of sequences defined in these Tables.

In yet another embodiment, the invention features antisense nucleic acid molecules and 2-5A chimera including sequences complementary to the substrate sequences shown in Tables III to VIII. Such nucleic acid molecules can include sequences as shown for the binding arms of the enzymatic nucleic acid molecules in Tables III to VIII. Similarly, triplex molecules can be provided targeted to the corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule may bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active nucleic acid molecule of the invention, for example an enzymatic nucleic acid molecule, contains an enzymatic center or core equivalent to those in the examples, and binding arms able to bind RNA such that cleavage at the target site occurs. Other sequences can be present which do not interfere with such cleavage. Thus, a core region can, for example, include one or more loop, stem-loop structure or linker, which does not prevent enzymatic activity. Thus, the underlined regions in the sequences in Tables III and IV can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by a sequence “X”, where X=5′-GCCGUUAGGC-3′ (SEQ ID NO 3190) or any other stem II region known in the art, or a nucleotide and/or non-nucleotide linker.

Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers may be present that do not interfere with the function of the nucleic acid molecule.

Feature X may be a linker of ≧2 nucleotides in length, preferably 3, 4, 5, 6, 7, 8, 9, 10, 15, 20, 26, 30, where the nucleotides may preferably be internally base-paired to form a stem of preferably ≧2 base pairs. Alternatively or in addition, X may be a non-nucleotide linker. In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, the non-nucleotide linker X is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound which can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave an RNA or DNA molecule.

In another aspect of the invention, ribozymes or antisense molecules that interact with target RNA molecules and inhibit CD20 (specifically CD20 gene) activity are expressed from transcription units inserted into DNA or RNA vectors. The recombinant vectors are preferably DNA plasmids or viral vectors. Ribozyme or antisense expressing viral vectors can be constructed based on, but not limited to, adeno-associated virus, retrovirus, adenovirus, or alphavirus. Preferably, the recombinant vectors capable of expressing the ribozymes or antisense are delivered as described herein, and persist in target cells. Alternatively, viral vectors can be used that provide for transient expression of ribozymes or antisense. Such vectors can be repeatedly administered as necessary. Once expressed, the ribozymes or antisense bind to the target RNA and inhibit its function or expression. Delivery of ribozyme or antisense expressing vectors can be systemic, such as by intravenous or intramuscular administration, by administration to target cells ex-planted from the patient followed by reintroduction into the patient, or by any other means that would allow for introduction into the desired target cell. Antisense DNA can be expressed via the use of a single stranded DNA intracellular expression vector.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the activity or stability of the nucleic acid molecule can be decreased (i.e., less than ten-fold), but the overall activity of the nucleic acid molecule is enhanced, in vivo.

The nucleic acid molecules of the instant invention, individually, or in combination or in conjunction with other drugs, can be used to treat diseases or conditions discussed above. For example, to treat a disease or condition associated with the levels of CD20, the patient may be treated, or other appropriate cells may be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules could be used in combination with one or more known therapeutic agents to treat lymphoma, leukemia, and arthropathy, including but not limited to B-cell lymphoma, low-grade or follicular non-Hodgkin's lymphoma (NHL), bulky low-grade or follicular NHL, lypmphocytic leukemia, HIV associated NHL, mantle-cell lymphoma (MCL), immunocytoma (IMC), small B-cell lymphocytic lymphoma, and immune thrombocytopenia, inflammatory arthropathy, and/or other disease states or conditions which respond to the modulation of CD20 expression.

In another preferred embodiment, the invention features nucleic acid-based inhibitors (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of genes (e.g., CD20) capable of progression and/or maintenance of lymphoma, leukemia, and arthropathy, including but not limited to B-cell lymphoma, low-grade or follicular non-Hodgkin's lymphoma (NHL), bulky low-grade or follicular NHL, lypmphocytic leukemia, HIV associated NHL, mantle-cell lymphoma (MCL), immunocytoma (IMC), small B-cell lymphocytic lymphoma, and immune thrombocytopenia, inflammatory arthropathy, and/or other disease states or conditions which respond to the modulation of CD20 expression.

In another aspect, the invention provides mammalian cells containing one or more nucleic acid molecules and/or expression vectors of this invention. The one or more nucleic acid molecules may independently be targeted to the same or different sites.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

Other features and advantages of the invention will be apparent from the following description of the preferred embodiments thereof, and from the claims.

### DESCRIPTION OF THE PREFERRED EMBODIMENTS

First the drawings will be described briefly.

### DRAWINGS

FIG. 1 shows the secondary structure model for seven different classes of enzymatic nucleic acid molecules. Arrow indicates the site of cleavage. --------- indicate the target sequence. Lines interspersed with dots are meant to indicate tertiary interactions. - is meant to indicate base-paired interaction. Group I Intron: P1-P9.0 represent various stem-loop structures (Cech et al., 1994, Nature Struc. Bio., 1, 273). RNase P (MIRNA): EGS represents external guide sequence (Forster et al., 1990, Science, 249, 783; Pace et al., 1990, J. Biol. Chem., 265, 3587). Group II Intron: 5′SS means 5′ splice site; 3′SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme:: I-IV are meant to indicate four stem-loop structures (Been et al., U.S. Pat. No. 5,625,047). Hammerhead Ribozyme:: I-III are meant to indicate three stem-loop structures; stems I-III can be of any length and may be symmetrical or asymmetrical (Usman et al., 1996, Curr. Op. Strilct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 may be covalently linked by one or more bases (i.e., r is ≧1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q”≧is 2 bases. The connecting loop can also be replaced with a non-nucleotide linker molecule. H refers to bases A, U, or C. Y refers to pyrimidine bases. “______” refers to a covalent bond. (Burke et al., 1996, Nucleic Acids & Mol. Biol., 10, 129; Chowrira et al., U.S. Pat. No. 5,631,359).

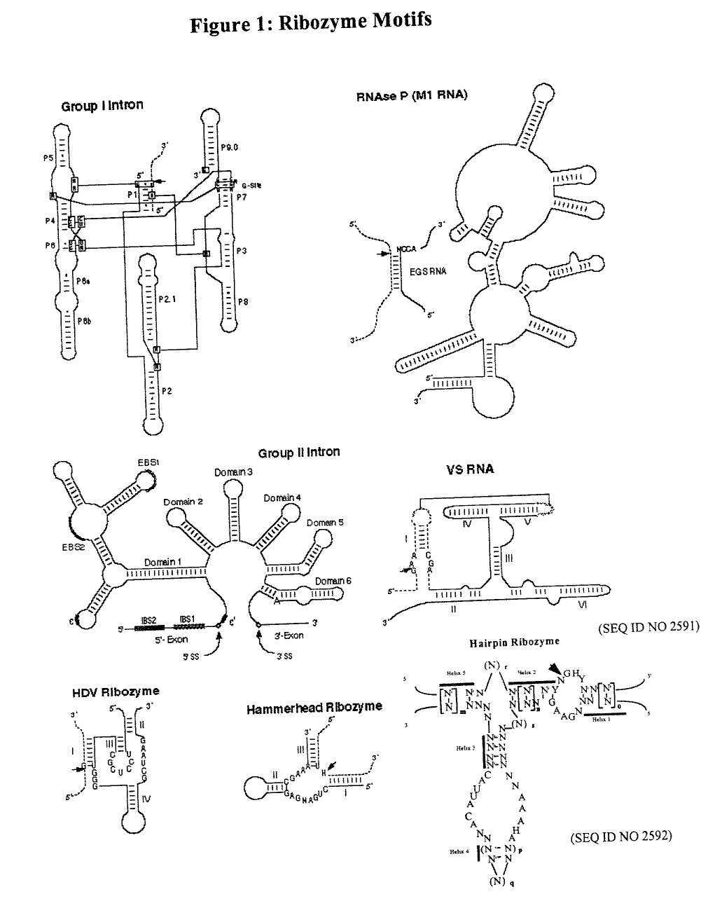

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120). N or n, represent independently a nucleotide which may be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

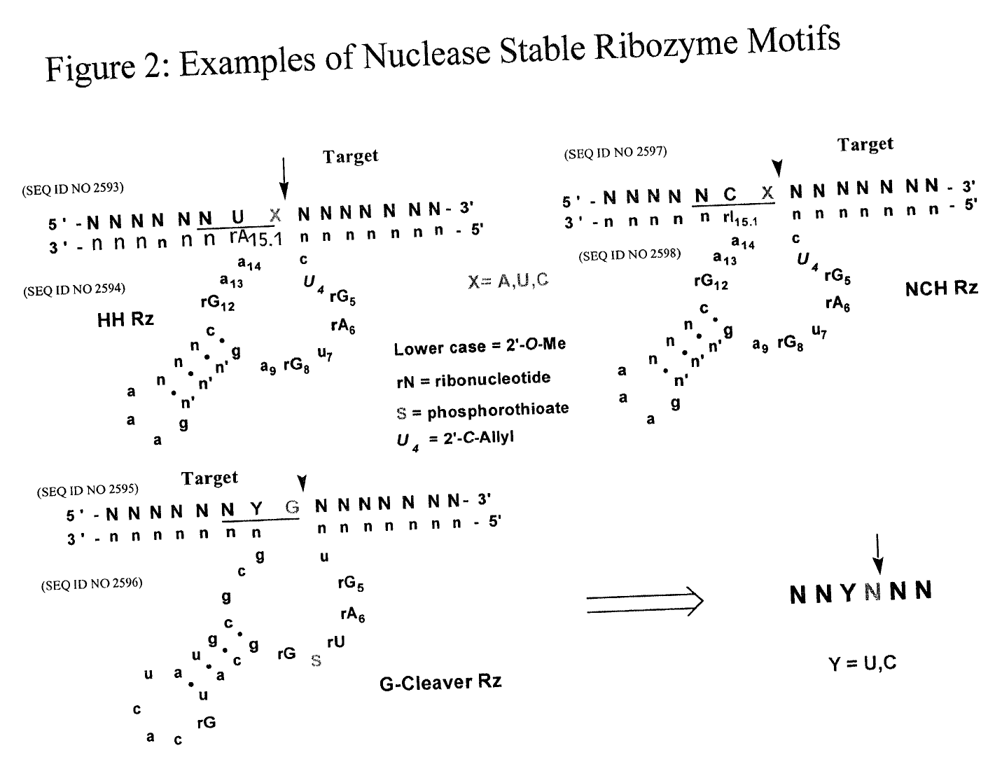

FIG. 3 shows an example of the Amberzyme ribozyme motif that is chemically stabilized (see, for example, Beigelman et al., International PCT publication No. WO 99/55857, incorporated by reference herein; also referred to as Class I Motif). The Amberzyme motif is a class of enzymatic nucleic molecules that do not require the presence of a ribonucleotide (2′-OH) group for its activity.

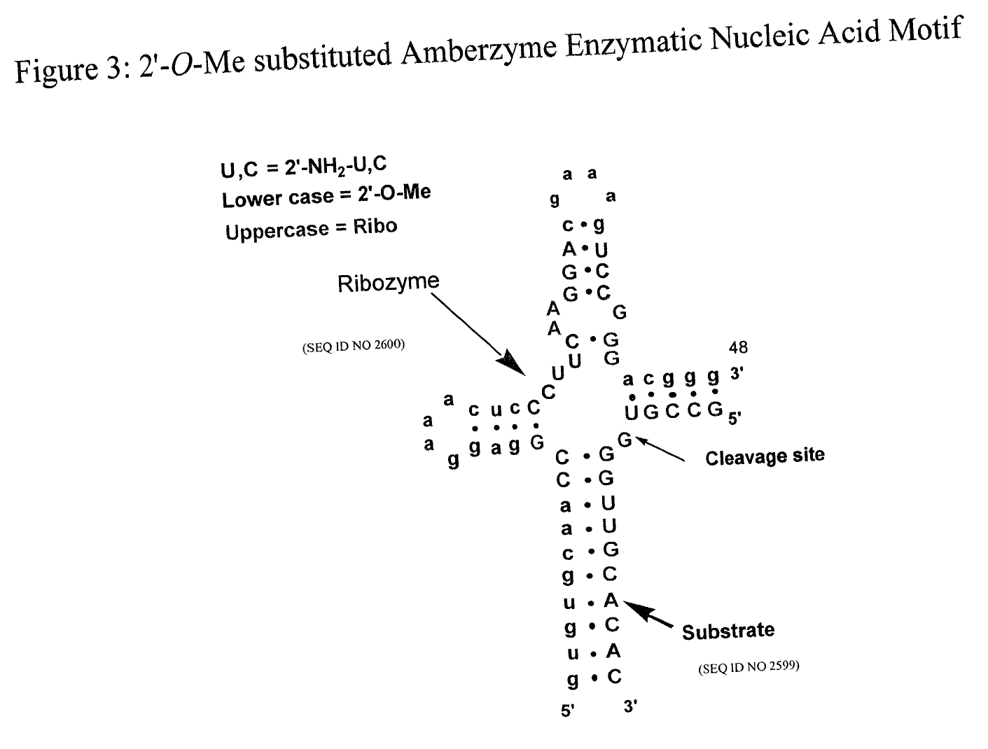

FIG. 4 shows an example of the Zinzyme A ribozyme motif that is chemically stabilized (Beigelman et al., International PCT publication No. WO 99/55857, incorporated by reference herein; also referred to as Class A or Class II Motif). The Zinzyme motif is a class of enzymatic nucleic molecules that do not require the presence of a ribonucleotide (2′-OH) group for its activity.

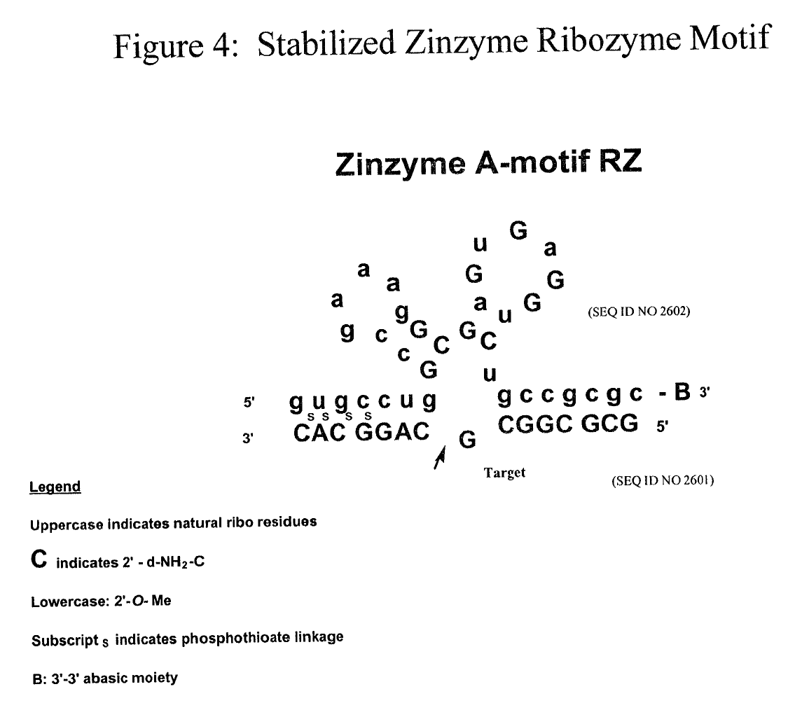

FIG. 5 shows an example of a DNAzyme motif described by Santoro et al., 1997, PNAS, 94, 4262.

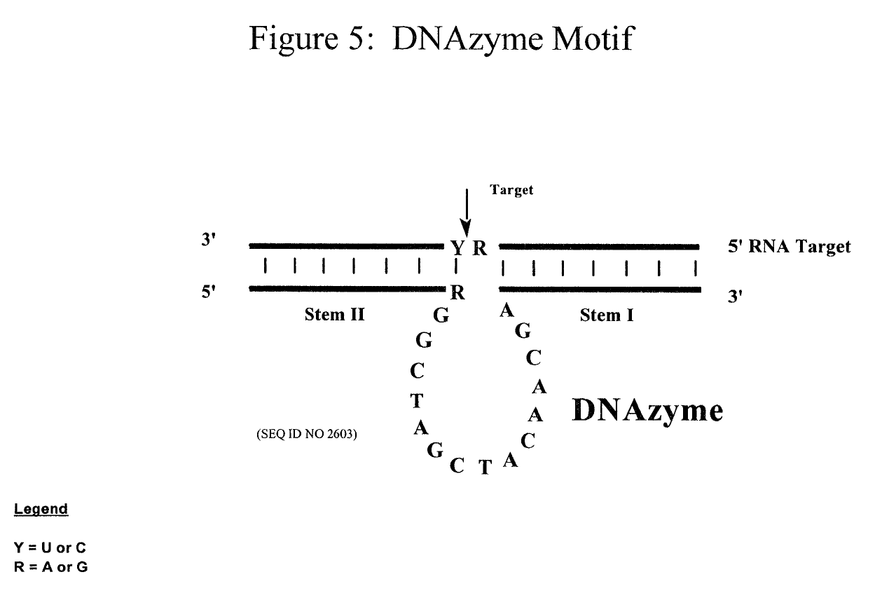

Mechanism of Action of Nucleic Acid Molecules of the Invention

Antisense: Antisense molecules can be modified or unmodified RNA, DNA, or mixed polymer oligonucleotides which primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. S. No. 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

Triplex Forming Oligonucleotides (TFO): Single stranded DNA may be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism may result in gene expression or cell death since binding may be irreversible (Mukhopadhyay & Roth, supra).

2-5A Antisense Chimera: The 2-5A system is an interferon mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures can be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

Enzymatic Nucleic Acid: Seven basic varieties of naturally occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al, 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent CD20 protein expression and can be used to treat disease or diagnose disease associated with the levels of CD20.

The enzymatic nature of a ribozyme has significant advantages, such as the concentration of ribozyme necessary to affect a therapeutic treatment is low. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a ribozyme.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; Jefferies et al., 17 Nucleic Acids Research 1371, 1989; and Santoro et al., 1997 supra).

Because of their sequence specificity, trans-cleaving ribozymes show promise as therapeutic agents for human disease (Usman & McSwiggen, 1995 Ann. Rep. Med. Chem. 30, 285-294; Christoffersen and Marr, 1995 J. Med. Chem. 38, 2023-2037). Ribozymes can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

The nucleic acid molecules of the instant invention are also referred to as GeneBloc™ reagents, which are essentially nucleic acid molecules (e.g.; ribozymes, antisense) capable of down-regulating gene expression.

GeneBlocs are modified oligonucleotides including ribozymes and modified antisense oligonucleotides that bind to and target specific mRNA molecules. Because GeneBlocs can be designed to target any specific mRNA, their potential applications are quite broad. Traditional antisense approaches have often relied heavily on the use of phosphorothioate modifications to enhance stability in biological samples, leading to a myriad of specificity problems stemming from non-specific protein binding and general cytotoxicity (Stein, 1995, Nature Medicine, 1, 1119). In contrast, GeneBlocs contain a number of modifications that confer nuclease resistance while making minimal use of phosphorothioate linkages, which reduces toxicity, increases binding affinity and minimizes non-specific effects compared with traditional antisense oligonucleotides. Similar reagents have recently been utilized successfully in various cell culture systems (Vassar, et al., 1999, Science, 286, 735) and in vivo (Jarvis et al., manuscript in preparation). In addition, novel cationic lipids can be utilized to enhance cellular uptake in the presence of serum. Since ribozymes and antisense oligonucleotides regulate gene expression at the RNA level, the ability to maintain a steady-state dose of GeneBloc over several days was important for target protein and phenotypic analysis. The advances in resistance to nuclease degradation and prolonged activity in vitro have supported the use of GeneBlocs in target validation applications.

Target Sites

Targets for useful ribozymes and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468. All of these publications are hereby incorporated by reference herein in their totality. Other examples include the following PCT applications, which concern inactivation of expression of disease-related genes: WO 95/23225, WO 95/13380, WO 94/02595, all of which are incorporated by reference herein. Rather than repeat the guidance provided in those documents here, specific examples of such methods are provided herein, not limiting to those in the art. Ribozymes and antisense to such targets are designed as described in those applications and synthesized to be tested in vitro and in vivo, as also described. The sequences of human CD20 RNAs were screened for optimal enzymatic nucleic acid and antisense target sites using a computer-folding algorithm. Antisense, hammerhead, DNAzyme, NCH, amberzyme, zinzyme, or G-Cleaver ribozyme binding/cleavage sites were identified. These sites are shown in Tables III to VIII (all sequences are 5′ to 3′ in the tables; underlined regions can be any sequence “X” or linker (X), the actual sequence is not relevant here). The nucleotide base position is noted in the Tables as that site to be cleaved by the designated type of enzymatic nucleic acid molecule. While human sequences can be screened and enzymatic nucleic acid molecule and/or antisense thereafter designed, as discussed in Stinchcomb et al., WO 95/23225, mouse targeted ribozymes can be useful to test efficacy of action of the enzymatic nucleic acid molecule and/or antisense prior to testing in humans.

Antisense, hammerhead, DNAzyme, NCH, amberzyme, zinzyme or G-Cleaver ribozyme binding/cleavage sites were identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core are eliminated from consideration. Varying binding arm lengths can be chosen to optimize activity.

Antisense, hammerhead, DNAzyme, NCH, amberzyme, zinzyme or G-Cleaver ribozyme binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; Wincott et al., 1995 Nucleic Acids Res. 23, 2677-2684; and Caruthers et al., 1992, Methods in Enzymology 211,3-19.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides (e.g.; antisense GeneBlocs) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methylimidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the antisense oligonucleotides is performed as follows. The polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the oligoribonucleotide, are dried to a white powder.

The method of synthesis used for normal RNA, including certain enzymatic nucleic acid molecules follows, the procedure as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684 and Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA.3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides) are synthesized by substituting a U for Gs and a U for A14 (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the examples described above including but not limited to 96-well format, all that is important is the ratio of chemicals used in the reaction.

Alternatively, the nucleic acid molecules of the present invention can be synthesized separately and joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; see Wincott et al., supra, the totality of which is hereby incorporated herein by reference) and are re-suspended in water.

The sequences of the ribozymes and antisense constructs that are chemically synthesized, useful in this study, are shown in Tables III to VIII. Those in the art will recognize that these sequences are representative only of many more such sequences where the enzymatic portion of the ribozyme (all but the binding arms) is altered to affect activity. The ribozyme and antisense construct sequences listed in Tables III to VIII may be formed of ribonucleotides or other nucleotides or non-nucleotides. Such ribozymes with enzymatic activity are equivalent to the ribozymes described specifically in the Tables.

Optimizing Activity of the Nucleic Acid Molecule of the Invention.

Chemically synthesizing nucleic acid molecules with modifications (base, sugar and/or phosphate) that prevent their degradation by serum ribonucleases may increase their potency (see e.g., Eckstein et al., International Publication No. WO 92/07065; Perrault et al, 1990 Nature 344, 565; Pieken et al., 1991, Science 253, 314; Usman and Cedergren, 1992, Trends in Biochem. Sci. 17, 334; Usman et al., International Publication No. WO 93/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules described herein. All these references are incorporated by reference herein. Modifications which enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modifications of nucleic acid molecules have been extensively described in the art (see Eckstein et al., International Publication PCT No. WO 92/07065; Perrault et al Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711, Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. S. No. 60/082,404 which was filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al, 1997, Bioorg. Med. Chem., 5, 1999-2010; all of these references are hereby incorporated by reference herein in their totalities). Such publications describe general methods and strategies to determine the location of incorporation of sugar, base and/or phosphate modifications and the like into ribozymes without inhibiting catalysis. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications may cause some toxicity. Therefore when designing nucleic acid molecules, the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid molecules are also generally more resistant to nucleases than unmodified nucleic acid molecules. Thus, in a cell and/or in vivo the activity may not be significantly lowered. Therapeutic nucleic acid molecules delivered exogenously must optimally be stable within cells until translation of the target RNA has been inhibited long enough to reduce the levels of the undesirable protein. This period of time varies between hours to days depending upon the disease state. Clearly, nucleic acid molecules must be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Use of these the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules can also include combinations of different types of nucleic acid molecules.

Therapeutic nucleic acid molecules (e.g., enzymatic nucleic acid molecules and antisense nucleic acid molecules) delivered exogenously should optimally be stable within cells until translation of the target RNA has been inhibited long enough to reduce the levels of the undesirable protein. This period of time varies between hours to days depending upon the disease state. In particular, these nucleic acid molecules should be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In yet another preferred embodiment, nucleic acid catalysts having chemical modifications which maintain or enhance enzymatic activity are provided. Such nucleic acid catalysts are also generally more resistant to nucleases than unmodified nucleic acid. Thus, in a cell and/or in vivo the activity may not be significantly lowered. As exemplified herein, such ribozymes are useful in a cell and/or in vivo even if activity over all is reduced 10 fold (Burgin et al, 1996, Biochemistry, 35, 14090). Such ribozymes herein are said to “maintain” the enzymatic activity of an all RNA ribozyme.

In another aspect, the nucleic acid molecules comprise a 5′ and/or a 3′-cap structure.

By “cap structure” is meant chemical modifications, which have been incorporated at either terminus of the oligonucleotide (see, for example, Wincott et al., WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminus (3′-cap) or may be present on both termini. In non-limiting examples, the 5′-cap is selected from the group consisting of inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moieiy; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details, see Wincott et al., International PCT publication No. WO 97/26270, incorporated by reference herein).

In yet another preferred embodiment, the 3′-cap is selected from a group consisting of 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details, see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group may be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO₂ or N(CH₃)₂, amino or SH.

Such alkyl groups can also include aryl, alkylaryl, carbocyclic aryl, heterocyclic aryl, amide and ester groups. An “aryl” group refers to an aromatic group which has at least one ring having a conjugated T electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In a preferred embodiment, the invention features modified ribozymes with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, (for more details, see Wincott et al., International PCT publication No. WO 97/26270).

By “unmodified nucleoside” is meant one of the bases adenine, cytosine, guanine, thymine, uracil joined to the 1′ carbon of beta-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′—NH₂ or 2′-O—NH₂, which may be modified or unmodified. Such modified groups are described, for example, in Eckstein et al., U.S. Pat. No. 5,672,695 and Matulic-Adamic et al., WO 98/28317, respectively, which are both incorporated by reference herein in their entireties.

Various modifications to nucleic acid (e.g., antisense and ribozyme) structure can be made to enhance the utility of these molecules. For example, such modifications enhance shelf-life, half-life in vitro, stability, and ease of introduction of such oligonucleotides to the target site, e.g., to enhance penetration of cellular membranes, and confer the ability to recognize and bind to targeted cells.

Use of these molecules can lead to better treatment of the disease progression by affording the possibility of combination therapies (e.g., multiple ribozymes targeted to different genes, ribozymes coupled with known small molecule inhibitors, or intermittent treatment with combinations of ribozymes (including different ribozyme motifs and/or other chemical or biological molecules). The treatment of patients with nucleic acid molecules can also include combinations of different types of nucleic acid molecules. For example, therapies can be devised which include a mixture of ribozymes (including different ribozyme motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid molecules can be directly delivered ex vivo to cells or tissues with or without the aforementioned vehicles. Alternatively, the nucleic acid/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions; suspensions for injectable administration, and other compositions known in the art.

The present invention also includes pharmaceutically acceptable formulations of the compounds described. These formulations include salts of the above compounds, e.g., acid addition salts, including salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example, oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes that lead to systemic absorption include, without limitations: intravenous, subcutaneous, intraperitoneal, inhalation, oral, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85) which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). All incorporated by reference herein. Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). All incorporated by reference herein. The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be added to the compositions. Suitable examples include sodium benzoate, sorbic acid and esters of p-hydroxybenzoic acid. In addition, antioxidants and suspending agents can be added.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention can also be administered to a patient in combination with other therapeutic compounds to increase the overall therapeutic effect. The use of multiple compounds to treat an indication may increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein).

In another aspect of the invention, RNA molecules of the present invention are preferably expressed from transcription units (see, for example, Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient followed by reintroduction into the patient, or by any other means that would allow for introduction into the desired target cell (for a review, see Couture et al., 1996, TIG., 12, 510).

In one aspect, the invention features an expression vector comprising a nucleic acid sequence encoding at least one of the nucleic acid molecules disclosed in the instant invention. The nucleic acid sequence encoding the nucleic acid molecule of the instant invention is operable linked in a manner which allows expression of that nucleic acid molecule.

In another aspect, the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector may optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depend on the nature of the gene regulatory sequences (enhancers, silencers, etc.) present nearby. Prokaryotic RNA polymerase promoters are also used, provided that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al, 1990, Mol. Cell. Biol., 10, 4529-37). All of these references are incorporated by reference herein.

Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al, 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; and Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; and Beigelman et al., International PCT Publication No. WO 96/18736; all of these publications are incorporated by reference herein. The above ribozyme transcription units can be incorporated into a variety of vectors for introduction into . mammalian cells, including but not restricted to, plasmid DNA vectors, viral DNA vectors (such as adenovirus or adeno-associated virus vectors), or viral RNA vectors (such as retroviral or alphavirus vectors) (for a review, see Couture and Stinchcomb, 1996, supra).

In yet another aspect, the invention features an expression vector comprising a nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another preferred embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule.

In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, hammerhead, DNAzyme, Inozyme, Amberzyme, Zinzyme, or G-Cleaver ribozyme molecules and binding/cleavage sites within CD20 RNA.

### Example 1

### Identification of Potential Target Sites in Human CD20 RNA

The sequence of human CD20 is screened for accessible sites using a computer-folding algorithm. Regions of the RNA are identified that do not form secondary folding structures. These regions contain potential ribozyme and/or antisense binding/cleavage sites. The sequences of these binding/cleavage sites are shown in Tables III-VIII.

### Example 2

### Selection of Enzymatic Nucleic Acid Cleavage Sites in Human CD20 RNA

Ribozyme target sites are chosen by analyzing sequences of Human CD20 (GenBank accession number: X07203) and prioritizing the sites on the basis of folding. Ribozymes are designed that could bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the ribozyme sequences fold into the appropriate secondary structure. Those ribozymes with unfavorable intramolecular interactions between the binding arms and the catalytic core are eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Ribozymes and Antisense for Efficient Cleavage and/or Blocking of CD20 RNA

Ribozymes and antisense constructs are designed to anneal to various sites in the RNA message. The binding arms of the ribozymes are complementary to the target site sequences described above, while the antisense constructs are fully complimentary to the target site sequences described above. The ribozymes and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al, supra, and made use of common nucleic acid protecting and coupling groups, such as dimethoxytrityl at the 5′-end, and phosphoramidites at the 3′-end. The average stepwise coupling yields were typically >98%.

Ribozymes and antisense constructs are also synthesized from DNA templates using bacteriophage T7 RNA polymerase (Milligan and Uhlenbeck, 1989, Methods Enzymol. 180, 51). Ribozymes and antisense constructs are purified by gel electrophoresis using general methods or are purified by high pressure liquid chromatography (HPLC; see Wincott et al., supra; the totality of which is hereby incorporated herein by reference) and are resuspended in water. The sequences of the chemically synthesized ribozymes and antisense constructs used in this study are shown below in Tables III-VIII.

### Example 4

### Ribozyme Cleavage of CD20 RNA Target in vitro

Ribozymes targeted to the human CD20 RNA are designed and synthesized as described above. These ribozymes can be tested for cleavage activity in vitro, for example, using the following procedure. The target sequences and the nucleotide location within the CD20 RNA are given in Tables III-VIII.

Cleavage Reactions: Full-length or partially full-length, internally-labeled target RNA for ribozyme cleavage assay is prepared by in vitro transcription in the presence of [a-³²P] CTP, passed over a G 50 Sephadex® column by spin chromatography and used as substrate RNA without further purification. Alternately, substrates are 5′-³²P-end labeled using T4 polynucleotide kinase enzyme. Assays are performed by pre-warming a 2× concentration of purified ribozyme in ribozyme cleavage buffer (50 mM Tris-HCl, pH 7.5 at 37° C., 10 mM MgCl₂) and the cleavage reaction was initiated by adding the 2× ribozyme mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM ribozyme, i.e., ribozyme excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imager® quantitation of bands representing the intact substrate and the cleavage products.

Cell Culture

Stacchini et al., 1999, Leuk. Res., 23(2), 127-126, describe the establishment of MEC1 and MEC2 cell lines derived from B-chronic lymphocytic leukemia in prolymphocytoid transformation. Matsuo et al., 1999, Leuk. Res., 23(6), 559-568, describe the establishment and characterization of a novel ALL-L3 cell line (BALM-18) in the study of apoptotic induction by anti-IgM and the inhibtion of apoptosis by bone marrow stroma cells. Schmetzer et al., 1998, Haematologia, 29(3), 195-205, describes the cloning and characterization of bone marrow cells from patients with acute lymphoid leukemia (ALL) in agar cultures. These cell lines express mature B cell markers including CD20, and can be used to study the modulation of CD20 expression using nucleic acid molecules of the instant invention.

Brandl et al., 1999, Exp. Hematol. (N.Y.), 27(8), 1264-1270, describe the use of bispecific antibody fragments with CD20×CD28 specificity to allow effective autologous and allogeneic T-cell activation against malignant cells in peripheral blood and bone marrow cultures from patients with B-cell lineage leukemia and lymphoma. A similar study using the nucleic acid molecules of the instant invention in place of antibody fragments can be used to evaluate the efficacy of nucleic acid molecules targeting CD20.

Animal Models

In order to evaluate the therapeutic potential of anti-CD20 ribozymes, several oncology models in rodent, rabbits and non-human primates may be utilized.

Human Xenograft models in Immunocomnromised Mice and/or Rats: The primary goal of these studies is to evaluate the effectiveness of anti-CD20 ribozyme therapy at reducing tumor burden and/or improving survival in animals with B-cell derived lymphoma. A variety of human lymphoma cell lines grow well as a subcutaneous solid tumor in unmanipulated immunocompromised mice or in nude mice subjected to sublethal irradiation. This allows for ease in measurement of tumor volumes. Cell lines that may be utilized include, but are not limited to: JeKo-1 (mantle cell lymphoma), Hs455 (Hodgkin's lymphoma), Hs 602 (cervical lymphoma) or CD 20+cells obtained from human patients. Human B lymphoid cells (BL2) may also be used to induce primary central nervous system lymphoma in nude rats (Jeon et al., 1998, Br. J. Haematol., 102(5), 1323-1326; Saini et al., 1999, J. Neurooncol., 43(2), 143-160).

Viral Induction of Lymphoma: These studies will evaluate the effectiveness of anti-CD20 ribozyme therapy at reducing tumor burden and/or improving survival in animals malignant lymphoma. Two animal models are available for inducing Epstein-Barr virus (EBV) related lymphomas. Rabbits can be inoculated orally with cell free pellets from cultured Si-IIA cells. These cells are a HTLV-II-transformed leukocyte cell line producing EBV. Malignant lymphomas developed after many weeks. Balb/c mice receiving subcutaneous transplants of human fetal nasopharyngeal mucosa infected with EBV can develop solid tumors provided that tumor promoters are administered concurrently. Subpopulations of tumor cells derived from such animals are CD20+. Tumor growth can be followed for up to 15 weeks post-inoculation (Koirala et al., 1997, Pathol. Int., 47(7), 442-448; Liu et al., 1998, J. Cancer. Res. Clin. Oncol., 124(10), 541-548).

Synyeneic Lymphoma Models in Mice: A variety of syngeneic murine lymphoma cell lines are available and can be grown in immunocompetent mice. Cell lines that may be utilized include, but are not limited to: V 38C13(B cell lymphoma), WEHI-279 or 231 (Non-secreting B-cell lymphomas) or P388D1 (lymphoma). Tumor burden and survival will be endpoints.

A genetically engineered mouse that spontaneously develops lymphoblastic lymphoma may also be utilized to verify activity of the anti-CD20 ribozyme. N:NIH(S)-bg-nu-xid mice develop a diffuse lymphoproliferative disorder by the age of 8 months. Lymph nodes are engorged with neoplastic lymphoblasts of B-cell origin (Weiner, 1992, Int. J. Cancer Suppl., 7, 63-66; Waggie et al., 1992, Lab Anim. Sci., 42(2), 375-377).

Indications

Particular conditions and disease states that can be associated with CD20 expression modulation include but are not limited to lymphoma, leukemia, and arthropathy. In particular, the nucleic acid molecules of the instant invention can be used to treat lymphoma, leukemia, and arthropathy including but not limited to B-cell lymphoma, low-grade or follicular non-Hodgkin's lymphoma (NHL), bulky low-grade or follicular NHL, lypmphocytic leukemia, HIV associated NHL, mantle-cell lymphoma (MCL), immunocytoma (IMC), small B-cell lymphocytic lymphoma, immune thrombocytopenia, and inflammatory arthropathy.

The present body of knowledge in CD20 research indicates the need for methods to assay CD20 activity and for compounds that can regulate CD20 expression for research, diagnostic, and therapeutic use.

Monoclonal antibodies and conjugates such as Bexxar, Rituxan, and Zevalin, chemotherapeutic agents such as CHOP (cyclophosphamide, doxorubicin, vincristine, and prednisone), immunomodulators, and radiation treatments are non-limiting examples of compounds and/or methods that can be combined with or used in conjunction with the nucleic acid molecules (e.g. ribozymes and antisense molecules) of the instant invention. Those skilled in the art will recognize that other drug compounds and therapies can be similarly and readily combined with the nucleic acid molecules of the instant invention (e.g. ribozymes and antisense molecules) and are, therefore, within the scope of the instant invention.

Diagnostic Uses

The nucleic acid molecules of this invention (e.g., ribozymes) can be used as diagnostic tools to examine genetic drift and mutations within diseased cells or to detect the presence of CD20 RNA in a cell. The close relationship between ribozyme activity and the structure of the target RNA allows the detection of mutations in any region of the molecule which alters the base-pairing and three-dimensional structure of the target RNA. By using multiple ribozymes described in this invention, one can map nucleotide changes which are important to RNA structure and function in vitro, as well as in cells and tissues. Cleavage of target RNAs with ribozymes can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention are well known in the art, and include detection of the presence of mRNAs associated with CD20-related condition. Such RNA is detected by determining the presence of a cleavage product after treatment with a ribozyme using standard methodology.

In a specific example, ribozymes which cleave only wild-type or mutant forms of the target RNA are used for the assay. The first ribozyme is used to identify wild-type RNA present in the sample and the second ribozyme is used to identify mutant RNA in the sample. As reaction controls, synthetic substrates of both wild-type and mutant RNA are cleaved by both ribozymes to demonstrate the relative ribozyme efficiencies in the reactions and the absence of cleavage of the “non-targeted” RNA species. The cleavage products from the synthetic substrates also serve to generate size markers for the analysis of wild-type and mutant RNAs in the sample population. Thus, each analysis requires two ribozymes, two substrates and one unknown sample, which are combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., CD20) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

Additional Uses

Potential usefulness of sequence-specific enzymatic nucleic acid molecules of the instant invention can have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs could be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the claims that follow.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate cleavage
products with 3′-OH and 5′-guanosine.
Additional protein cofactors required in some cases to help folding and
maintainance of the active structure.
Over 300 known members of this class. Found as an intervening sequence in
Tetrahymena thermophila rRNA, fungal mitochondria, chloroplasts, phage T4, blue-
green algae, and others.
Major structural features largely established through phylogenetic comparisons,
mutagenesis, and biochemical studies [ⁱ,ⁱⁱ].
Complete kinetic framework established for one ribozyme [ⁱⁱⁱ,^{iv},^{v},^{vi}].
Studies of ribozyme folding and substrate docking underway [^{vii},^{Viii},^{ix}].
Chemical modification investigation of important residues well established [^{x},^{xi}].
The small (4-6 nt) binding site may make this ribozyme too non-specific for
targeted RNA cleavage, however, the Tetrahymena group I intron has been used
to repair a “defective” □-galactosidase message by the ligation of new □-
galactosidase sequences onto the defective message [^{xii}].
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii]}.
Reaction mechanism: possible attack by M²⁺-OH to generate cleavage products
with 3′-OH and 5′-phosphate.
RNAse P is found throughout the prokaryotes and eukaryotes. The RNA subunit has been
sequenced from bacteria, yeast, rodents, and primates.
Recruitment of endogenous RNAse P for therapeutic applications is possible
through hybridization of an External Guide Sequence (EGS) to the target RNA
[^{xiv},^{xv}]
Important phosphate and 2′ OH contacts recently identified [^{xvi},^{xvii}]
Group II Introns
Size: >1000 nucleotides.
Trans cleavage of target RNAs recently demonstrated [^{xviii},^{xix}].
Sequence requirements not fully determined.
Reaction mechanism: 2′-OH of an internal adenosine generates cleavage products
with 3′-OH and a “lariat” RNA containing a 3′-5′ and a 2′-5′ branch point.
Only natural ribozyme with demonstrated participation in DNA cleavage [^{xx},^{xxi}] in
addition to RNA cleavage and ligation.
Major structural features largely established through phylogenetic comparisons
[^{xxii}]
Important 2′ OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′ to the scissile bond to generate cleavage
products with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate cleavage
products with 2′,3′-cyclic phosphate and 5′-OH ends.
14 known members of this class. Found in a number of plant pathogens
(virusoids) that use RNA as the infectious agent.
Essential structural features largely defined, including 2 crystal structures [^{xxvi},^{xxvii}]
Minimal ligation activity demonstrated (for engineering through in vitro selection)
[^{xxviii}]
Complete kinetic framework established for two or more ribozymes [^{xxix}].
Chemical modification investigation of important residues well established [^{xxx}].
Hairpin Ribozyme
Size: ˜50 nucleotides.
Requires the target sequence GUC immediately 3′ of the cleavage site.
Binds 4-6 nucleotides at the 5′-side of the cleavage site and a variable number to
the 3′-side of the cleavage site.
Reaction mechanism: attack by 2′-OH 5′ to the scissile bond to generate cleavage
products with 2′,3′-cyclic phosphate and 5′-OH ends.
3 known members of this class. Found in three plant pathogen (satellite RNAs of
the tobacco ringspot virus, arabis mosaic virus and chicory yellow mottle virus)
which uses RNA as the infectious agent.
Essential structural features largely defined [^{xxxi},^{xxxii},^{xxxiii},^{xxxiv}]
Ligation activity (in addition to cleavage activity) makes ribozyme amenable to
engineering through in vitro selection [^{xxxv}]
Complete kinetic framework established for one ribozyme [^{xxxvi}].
Chemical modification investigation of important residues begun [^{xxxvii},^{xxxviii}].
Hepatitis Delta Virus (HDV) Ribozyme
Size: ˜60 nucleotides.
Trans cleavage of target RNAs demonstrated [^{xxxix}].
Binding sites and structural requirements not fully determined, although no
sequences 5′ of cleavage site are required. Folded ribozyme contains a pseudoknot
structure [^{xl}]
Reaction mechanism: attack by 2′-OH 5′ to the scissile bond to generate cleavage
products with 2′,3′-cyclic phosphate and 5′-OH ends.
Only 2 known members of this class. Found in human HDV.
^{xli}Circular form of HDV is active and shows increased nuclease stability [^{xlii}]
ⁱMichel, Francois; Westhof, Eric. Slippery substrates. Nat. Struct. Biol. (1994), 1(1), 5-7.
ⁱⁱLisacek, Frederique; Diaz, Yolande; Michel, Francois. Automatic identification of group I intron cores in genomic DNA sequences. J. Mol. Biol. (1994), 235(4), 1206-17.
ⁱⁱⁱHerschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.
^{iv}Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
^{v}Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
^{vi}Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.
^{vii}Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
^{viii}Banerjee, Aloke Raj; Turner, Douglas H.. The time dependence of chemical modification reveals slow steps in the folding of a group I ribozyme. Biochemistry (1995), 34(19), 6504-12.
^{ix}Zarrinkar, Patrick P.; Williamson, James R.. The P9.1-P9.2 peripheral extension helps guide folding of the Tetrahymena ribozyme. Nucleic Acids Res. (1996), 24(5), 854-8.
^{x}Strobel, Scott A.; Cech, Thomas R.. Minor groove recognition of the conserved G.cntdot.U pair at the Tetrahymena ribozyme reaction site. Science (Washington, D.C.) (1995), 267(5198), 675-9.
^{xi}Strobel, Scott A.; Cech, Thomas R.. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
^{xii}Sullenger, Bruce A.; Cech, Thomas R.. Ribozyme-mediated repair of defective mRNA by targeted trans-splicing. Nature (London) (1994), 371 (6498), 619-22.
^{xiii}Robertson, H.D.; Altman, S.; Smith, J.D. J. Biol. Chem., 247, 5243-5251 (1972).
^{xiv}Forster, Anthony C.; Altman, Sidney. External guide sequences for an RNA enzyme. Science (Washington, D.C., 1883-) (1990), 249(4970), 783-6.
^{xv}Yuan, Y.; Hwang, E. S.; Altman, S. Targeted cleavage of mRNA by human RNase P. Proc. Natl. Acad. Sci. USA (1992) 89, 8006-10.
^{xvi}Harris, Michael E.; Pace, Norman R.. Identification of phosphates involved in catalysis by the ribozyme RNase P RNA. RNA (1995), 1(2), 210-18.
^{xvii}Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U.S.A. (1995), 92(26), 12510-14.
^{xviii}Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
^{xix}Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
^{xx}Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83(4), 529-38.
^{xxi}Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
^{xxii}Michel, Francois; Ferat, Jean Luc. Structure and activities of group II introns. Annu. Rev. Biochem. (1995), 64, 435-61.
^{xxiii}Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.
^{xxiv}Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
^{xxv}Guo, Hans C.T.; Collins, Richard A.. Efficient trans-cleavage of a stem-loop RNA substrate by a ribozyme derived from Neurospora VS RNA. EMBO J. (1995), 14(2), 368-76.
^{xxvi}Scott, W.G., Finch, J.T., Aaron, K. The crystal structure of an all RNA hammerhead ribozyme: Aproposed mechanism for RNA catalytic cleavage. Cell, (1995), 81, 991-1002.
^{xxvii}McKay, Structure and function of the hammerhead ribozyme: an unfinished story. RNA, (1996), 2, 395-403.
^{xxviii}Long, D., Uhlenbeck, O., Hertel, K. Ligation with hammerhead ribozymes. U.S. Pat. No. 5,633,133.
^{xxix}Hertel, K.J., Herschlag, D., Uhlenbeck, O. A kinetic and thermodynamic framework for the hammerhead ribozyme reaction. Biochemistry, (1994) 33, 3374-3385. Beigelman, L., et aL, Chemical modifications of hammerhead ribozymes. J. Biol. Chem., (1995) 270, 25702-25708.
^{xxx}Beigelman, L., et al, Chemical modifications of hammerhead ribozymes. J. Biol. Chem., (1995) 270, 25702-25708.
^{xxxi}Hampel, Arnold; Tritz, Richard; Hicks, Margaret; Cruz, Philip. ‘Hairpin’ catalytic RNA model: evidence for helixes and sequence requirement for substrate RNA. Nucleic Acids Res. (1990), 18(2), 299-304.
^{xxxii}Chowrira, Bharat M.; Berzal-Herranz, Alfredo; Burke, John M.. Novel guanosine requirement for catalysis by the hairpin ribozyme. Nature (London) (1991), 354(6351), 320-2.
^{xxxiii}Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J. (1993), 12(6), 2567-73.
^{xxxiv}Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.
^{xxxv}Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.
^{xxxvi}Hegg, Lisa A.; Fedor, Martha J.. Kinetics and Thermodynamics of Intermolecular Catalysis by Hairpin Ribozymes. Biochemistry (1995), 34(48), 15813-28.
^{xxxvii}Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34(12), 4068-76.
^{xxxviii}Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.
^{xxxix}Perrotta, Anne T.; Been, Michael D.. Cleavage of oligoribonucleotides by a ribozyme derived from the hepatitis .delta. virus RNA sequence. Biochemistry (1992), 31(1), 16-21.
^{xl}Perrotta, Anne T.; Been, Michael D.. A pseudoknot-like structure required for efficient self-cleavage of hepatitis delta virus RNA. Nature (London) (1991), 350(6317), 434-6.
^{xli}
^{xlii}Puttaraju, M.; Perrotta, Anne T.; Been, Michael D.. A circular trans-acting hepatitis delta virus ribozyme. Nucleic Acids Res. (1993), 21(18), 4253-8.

TABLE II
A. 2.5 μmol Synthesis Cycle ABI 394 Instrument

| | | | | | Wait Time* 2′-O- | |
|---|---|---|---|---|---|---|
| Reagent | Equivalents | Amount | | Wait Time* DNA | methyl | Wait Time* RNA |
| Phosphoramidites | 6.5 | 163 | μL | 45 sec | 2.5 min | 7.5 min |
| S-Ethyl Tetrazole | 23.8 | 238 | μL | 45 sec | 2.5 min | 7.5 min |
| Acetic Anhydride | 100 | 233 | μL | 5 sec | 5 sec | 5 sec |
| N-Methyl | 186 | 233 | μL | 5 sec | 5 sec | 5 sec |
| Imidazole | | | | | | |
| TCA | 176 | 2.3 | mL | 21 sec | 21 sec | 21 sec |
| Iodine | 11.2 | 1.7 | mL | 45 sec | 45 sec | 45 sec |
| Beaucage | 12.9 | 645 | μL | 100 sec | 300 sec | 300 sec |
| Acetonitrile | NA | 6.67 | mL | NA | NA | NA |

B. 0.2 μmol Synthesis Cycle ABI 394 Instrument

| | | | | | Wait Time* 2′-O- | |
|---|---|---|---|---|---|---|
| Reagent | Equivalents | Amount | | Wait Time* DNA | methyl | Wait Time* RNA |
| Phosphoramidites | 15 | 31 | μL | 45 sec | 233 sec | 465 sec |
| S-Ethyl Tetrazole | 38.7 | 31 | μL | 45 sec | 233 min | 465 sec |
| Acetic Anhydride | 655 | 124 | μL | 5 sec | 5 sec | 5 sec |
| N-Methyl | 1245 | 124 | μL | 5 sec | 5 sec | 5 sec |
| Imidazole | | | | | | |
| TCA | 700 | 732 | μL | 10 sec | 10 sec | 10 sec |
| Iodine | 20.6 | 244 | μL | 15 sec | 15 sec | 15 sec |
| Beaucage | 7.7 | 232 | μL | 100 sec | 300 sec | 300 sec |
| Acetonitrile | NA | 2.64 | mL | NA | NA | NA |

C. 0.2 μmol Synthesis Cycle 96 well Instrument

| | Equivalents: | | | | | |
|---|---|---|---|---|---|---|
| | DNA/ | | | | | |
| | 2′-O-methyl/ | Amount: DNA/2′-O- | | | Wait Time* 2′-O- | |
| Reagent | Ribo | methyl/Ribo | | Wait Time* DNA | methyl | Wait Time* Ribo |
| Phosphoramidites | 22/33/66 | 40/60/120 | μL | 60 sec | 180 sec | 360 sec |
| S-Ethyl Tetrazole | 70/105/210 | 40/60/120 | μL | 60 sec | 180 min | 360 sec |
| Acetic Anhydride | 265/265/265 | 50/50/50 | μL | 10 sec | 10 sec | 10 sec |
| N-Methyl | 502/502/502 | 50/50/50 | μL | 10 sec | 10 sec | 10 sec |
| Imidazole | | | | | | |
| TCA | 238/475/475 | 250/500/500 | μL | 15 sec | 15 sec | 15 sec |
| Iodine | 6.8/6.8/6.8 | 80/80/80 | μL | 30 sec | 30 sec | 30 sec |
| Beaucage | 34/51/51 | 80/120/120 | | 100 sec | 200 sec | 200 sec |
| Acetonitrile | NA | 1150/1150/1150 | μL | NA | NA | NA |

*Wait time does not include contact time during delivery.

TABLE III
Human CD20 Hammerhead Ribozyme and Substrate Sequence

| | | | | Rz Seq |
|---|---|---|---|---|
| Pos | Substrate | Seq ID | Ribozyme | ID |
| 23 | ACUGAACU C CGCAGCUA | 1 | UAGCUGCG CUGAUGAG GCCGUUAGGC CGAA AGUUCAGU | 1093 |
| 31 | CCGCAGCU A GCAUCCAA | 2 | UUGGAUGC CUGAUGAG GCCGUUAGGC CGAA AGCUGCGG | 1094 |
| 36 | GCUAGCAU C CAAAUCAG | 3 | CUGAUUUG CUGAUGAG GCCGUUAGGC CGAA AUGCUAGC | 1095 |
| 42 | AUCCAAAU C AGCCCUUG | 4 | CAAGGGCU CUGAUGAG GCCGUUAGGC CGAA AUUUGGAU | 1096 |
| 49 | UCAGCCCU U GAGAUUUG | 5 | CAAAUCUC CUGAUGAG GCCGUUAGGC CGAA AGGGCUGA | 1097 |
| 55 | CUUGAGAU U UGAGGCCU | 6 | AGGCCUCA CUGAUGAG GCCGUUAGGC CGAA AUCUCAAG | 1098 |
| 56 | UUGAGAUU U GAGGCCUU | 7 | AAGGCCUC CUGAUGAG GCCGUUAGGC CGAA AAUCUCAA | 1099 |
| 64 | UGAGGCCU U GGAGACUC | 8 | GAGUCUCC CUGAUGAG GCCGUUAGGC CGAA AGGCCUCA | 1100 |
| 72 | UGGAGACU C AGGAGUUU | 9 | AAACUCCU CUGAUGAG GCCGUUAGGC CGAA AGUCUCCA | 1101 |
| 79 | UCAGGAGU U UUGAGAGC | 10 | GCUCUCAA CUGAUGAG GCCGUUAGGC CGAA ACUCCUGA | 1102 |
| 80 | CAGGAGUU U UGAGAGCA | 11 | UGCUCUCA CUGAUGAG GCCGUUAGGC CGAA AACUCCUG | 1103 |
| 81 | AGGAGUUU U GAGAGCAA | 12 | UUGCUCUC CUGAUGAG GCCGUUAGGC CGAA AAACUCCU | 1104 |
| 109 | CCAGAAAU U CAGUAAAU | 13 | AUUUACUG CUGAUGAG GCCGUUAGGC CGAA AUUUCUGG | 1105 |
| 110 | CAGAAAUU C AGUAAAUG | 14 | CAUUUACU CUGAUGAG GCCGUUAGGC CGAA AAUUUCUG | 1106 |
| 114 | AAUUCAGU A AAUGGGAC | 15 | GUCCCAUU CUGAUGAG GCCGUUAGGC CGAA ACUGAAUU | 1107 |
| 124 | AUGGGACU U UCCUGGCA | 16 | UGCCAGGA CUGAUGAG GCCGUUAGGC CGAA AGUCCCAU | 1108 |
| 125 | UGGGACUU U CCUGGCAG | 17 | CUGCCAGG CUGAUGAG GCCGUUAGGC CGAA AAGUCCCA | 1109 |
| 126 | GGGACUUU C CUGGCAGA | 18 | UCUGCCAG CUGAUGAG GCCGUUAGGC CGAA AAAGUCCC | 1110 |
| 151 | AAGGCCCU A UUGCUAUG | 19 | CAUAGCAA CUGAUGAG GCCGUUAGGC CGAA AGGGCCUU | 1111 |
| 153 | GGCCCUAU U GCUAUGCA | 20 | UGCAUAGC CUGAUGAG GCCGUUAGGC CGAA AUAGGGCC | 1112 |
| 157 | CUAUUGCU A UGCAAUCU | 21 | AGAUUGCA CUGAUGAG GCCGUUAGGC CGAA AGCAAUAG | 1113 |
| 164 | UAUGCAAU C UGGUCCAA | 22 | UUGGACCA CUGAUGAG GCCGUUAGGC CGAA AUUGCAUA | 1114 |
| 169 | AAUCUGGU C CAAAACCA | 23 | UGGUUUUG CUGAUGAG GCCGUUAGGC CGAA ACCAGAUG | 1115 |
| 180 | AAACCACU C UUCAGGAG | 24 | CUCCUGAA CUGAUGAG GCCGUUAGGC CGAA AGUGGUUU | 1116 |
| 182 | ACCACUCU U CAGGAGGA | 25 | UCCUCCUG CUGAUGAG GCCGUUAGGC CGAA AGAGUGGU | 1117 |
| 183 | CCACUCUU C AGGAGGAU | 26 | AUCCUCCU CUGAUGAG GCCGUUAGGC CGAA AAGAGUGG | 1118 |
| 194 | GAGGAUGU C UUCACUGG | 27 | CCAGUGAA CUGAUGAG GCCGUUAGGC CGAA ACAUCCUC | 1119 |
| 196 | GGAUGUCU U CACUGGUG | 28 | CACCAGUG CUGAUGAG GCCGUUAGGC CGAA AGACAUCC | 1120 |
| 197 | GAUGUCUU C ACUGGUGG | 29 | CCACCAGU CUGAUGAG GCCGUUAGGC CGAA AAGACAUC | 1121 |
| 221 | GCAAAGCU U CUUCAUGA | 30 | UCAUGAAG CUGAUGAG GCCGUUAGGC CGAA AGCUUUGC | 1122 |
| 222 | CAAAGCUU C UUCAUGAG | 31 | CUCAUGAA CUGAUGAG GCCGUUAGGC CGAA AAGCUUUG | 1123 |
| 224 | AAGCUUCU U CAUGAGGG | 32 | CCCUCAUG CUGAUGAG GCCGUUAGGC CGAA AGAAGCUU | 1124 |
| 225 | AGCUUCUU C AUGAGGGA | 33 | UCCCUCAU CUGAUGAG GCCGUUAGGC CGAA AAGAAGCU | 1125 |
| 236 | GAGGGAAU C UAAGACUU | 34 | AAGUCUUA CUGAUGAG GCCGUUAGGC CGAA AUUCCCUC | 1126 |
| 238 | GGGAAUCU A AGACUUUG | 35 | CAAAGUCU CUGAUGAG GCCGUUAGGC CGAA AGAUUCCC | 1127 |
| 244 | CUAAGACU U UGGGGGCU | 36 | AGCCCCCA CUGAUGAG GCCGUUAGGC CGAA AGUCUUAG | 1128 |
| 245 | UAAGACUU U GGGGGCUG | 37 | CAGCCCCC CUGAUGAG GCCGUUAGGC CGAA AAGUCUUA | 1129 |
| 255 | GGGGCUGU C CAGAUUAU | 38 | AUAAUCUG CUGAUGAG GCCGUUAGGC CGAA ACAGCCCC | 1130 |
| 261 | GUCCAGAU U AUGAAUGG | 39 | CCAUUCAU CUGAUGAG GCCGUUAGGC CGAA AUCUGGAC | 1131 |
| 262 | UCCAGAUU A UGAAUGGG | 40 | CCCAUUCA CUGAUGAG GCCGUUAGGC CGAA AAUCUGGA | 1132 |
| 273 | AAUGGGCU C UUCCACAU | 41 | AUGUGGAA CUGAUGAG GCCGUUAGGC CGAA AGCCCAUU | 1133 |
| 275 | UGGGCUCU U CCACAUUG | 42 | CAAUGUGG CUGAUGAG GCCGUUAGGC CGAA AGAGCCCA | 1134 |
| 276 | GGGCUCUU C CACAUUGC | 43 | GCAAUGUG CUGAUGAG GCCGUUAGGC CGAA AAGAGCCC | 1135 |
| 282 | UUCCACAU U GCCCUGGG | 44 | CCCAGGGC CUGAUGAG GCCGUUAGGC CGAA AUGUGGAA | 1136 |
| 295 | UGGGGGGU C UUCUGAUG | 45 | CAUCAGAA CUGAUGAG GCCGUUAGGC CGAA ACCCCCCA | 1137 |
| 297 | GGGGGUCU U CUGAUGAU | 46 | AUCAUCAG CUGAUGAG GCCGUUAGGC CGAA AGACCCCC | 1138 |
| 298 | GGGGUCUU C UGAUGAUC | 47 | GAUCAUCA CUGAUGAG GCCGUUAGGC CGAA AAGACCCC | 1139 |
| 306 | CUGAUGAU C CCAGCAGG | 48 | CCUGCUGG CUGAUGAG GCCGUUAGGC CGAA AUCAUCAG | 1140 |
| 318 | GCAGGGAU C UAUGCACC | 49 | GGUGCAUA CUGAUGAG GCCGUUAGGC CGAA AUCCCUGC | 1141 |
| 320 | AGGGAUCU A UGCACCCA | 50 | UGGGUGCA CUGAUGAG GCCGUUAGGC CGAA AGAUCCCU | 1142 |
| 330 | GCACCCAU C UGUGUGAC | 51 | GUCACACA CUGAUGAG GCCGUUAGGC CGAA AUGGGUGC | 1143 |
| 347 | UGUGUGGU A CCCUCUCU | 52 | AGAGAGGG CUGAUGAG GCCGUUAGGC CGAA ACCACACA | 1144 |
| 352 | GGUACCCU C UCUGGGGA | 53 | UCCCCAGA CUGAUGAG GCCGUUAGGC CGAA AGGGUACC | 1145 |
| 354 | UACCCUCU C UGGGGAGG | 54 | CCUCCCCA CUGAUGAG GCCGUUAGGC CGAA AGAGGGUA | 1146 |
| 366 | GGAGGCAU U AUGUAUAU | 55 | AUAUACAU CUGAUGAG GCCGUUAGGC CGAA AUGCCUCC | 1147 |
| 367 | GAGGCAUU A UGUAUAUU | 56 | AAUAUACA CUGAUGAG GCCGUUAGGC CGAA AAUGCCUC | 1148 |
| 371 | CAUUAUGU A UAUUAUUU | 57 | AAAUAAUA CUGAUGAG GCCGUUAGGC CGAA ACAUAAUG | 1149 |
| 373 | UUAUGUAU A UUAUUUCC | 58 | GGAAAUAA CUGAUGAG GCCGUUAGGC CGAA AUACAUAA | 1150 |
| 375 | AUGUAUAU U AUUUCCGG | 59 | CCGGAAAU CUGAUGAG GCCGUUAGGC CGAA AUAUACAU | 1151 |
| 376 | UGUAUAUU A UUUCCGGA | 60 | UCCGGAAA CUGAUGAG GCCGUUAGGC CGAA PAUAUACA | 1152 |
| 378 | UAUAUUAU U UCCGGAUC | 61 | GAUCCGGA CUGAUGAG GCCGUUAGGC CGAA AUAAUAUA | 1153 |
| 379 | AUAUUAUU U CCGGAUCA | 62 | UGAUCCGG CUGAUGAG GCCGUUAGGC CGAA AAUAAUAU | 1154 |
| 380 | UAUUAUUU C CGGAUCAC | 63 | GUGAUCCG CUGAUGAG GCCGUUAGGC CGAA AAAUAAUA | 1155 |
| 386 | UUCCGGAU C ACUCCUGG | 64 | CCAGGAGU CUGAUGAG GCCGUUAGGC CGAA AUCCGGAA | 1156 |
| 390 | GGAUCACU C CUGGCAGC | 65 | GCUGCCAG CUGAUGAG GCCGUUAGGC CGAA AGUGAUCC | 1157 |
| 413 | GAAAAACU C CAGGAAGU | 66 | ACUUCCUG CUGAUGAG GCCGUUAGGC CGAA AGUUUUUC | 1158 |
| 424 | GGAAGUGU U UGGUCAAA | 67 | UUUGACCA CUGAUGAG GCCGUUAGGC CGAA ACACUUCC | 1159 |
| 425 | GAAGUGUU U GGUCAAAG | 68 | CUUUGACC CUGAUGAG GCCGUUAGGC CGAA AACACUUC | 1160 |
| 429 | UGUUUGGU C AAAGGAAA | 69 | UUUCCUUU CUGAUGAG GCCGUUAGGC CGAA ACCAAACA | 1161 |
| 444 | AAAAUGAU A AUGAAUUC | 70 | GAAUUCAU CUGAUGAG GCCGUUAGGC CGAA AUCAUUUU | 1162 |
| 451 | UAAUGAAU U CAUUGAGC | 71 | GCUCAAUG CUGAUGAG GCCGUUAGGC CGAA AUUCAUUA | 1163 |
| 452 | AAUGAAUU C AUUGAGCC | 72 | GGCUCAAU CUGAUGAG GCCGUUAGGC CGAA AAUUCAUU | 1164 |
| 455 | GAAUUCAU U GAGCCUCU | 73 | AGAGGCUC CUGAUGAG GCCGUUAGGC CGAA AUGAAUUC | 1165 |
| 462 | UUGAGCCU C UUUGCUGC | 74 | GCAGCAAA CUGAUGAG GCCGUUAGGC CGAA AGGCUCAA | 1166 |
| 464 | GAGCCUCU U UGCUGCCA | 75 | UGGCAGCA CUGAUGAG GCCGUUAGGC CGAA AGAGGCUC | 1167 |
| 465 | AGCCUCUU U GCUGCCAU | 76 | AUGGCAGC CUGAUGAG GCCGUUAGGC CGAA AAGAGGCU | 1168 |
| 474 | GCUGCCAU U UCUGGAAU | 77 | AUUCCAGA CUGAUGAG GCCGUUAGGC CGAA AUGGCAGC | 1169 |
| 475 | CUGCCAUU U CUGGAAUG | 78 | CAUUCCAG CUGAUGAG GCCGUUAGGC CGAA AAUGGCAG | 1170 |
| 476 | UGCCAUUU C UGGAAUGA | 79 | UCAUUCCA CUGAUGAG GCCGUUAGGC CGAA AAAUGGCA | 1171 |
| 486 | GGAAUGAU U CUUUCAAU | 80 | AUUGAAAG CUGAUGAG GCCGUUAGGC CGAA AUCAUUCC | 1172 |
| 487 | GAAUGAUU C UUUCAAUC | 81 | GAUUGAAA CUGAUGAG GCCGUUAGGC CGAA AAUCAUUC | 1173 |
| 489 | AUGAUUCU U UCAAUCAU | 82 | AUGAUUGA CUGAUGAG GCCGUUAGGC CGAA AGAAUCAU | 1174 |
| 490 | UGAUUCUU U CAAUCAUG | 83 | CAUGAUUG CUGAUGAG GCCGUUAGGC CGAA AAGAAUCA | 1175 |
| 491 | GAUUCUUU C AAUCAUGG | 84 | CCAUGAUU CUGAUGAG GCCGUUAGGC CGAA AAAGAAUC | 1176 |
| 495 | CUUUCAAU C AUGGACAU | 85 | AUGUCCAU CUGAUGAG GCCGUUAGGC CGAA AUUGAAAG | 1177 |
| 504 | AUGGACAU A CUUAAUAU | 86 | AUAUUAAG CUGAUGAG GCCGUUAGGC CGAA AUGUCCAU | 1178 |
| 507 | GACAUACU U AAUAUUAA | 87 | UUAAUAUU CUGAUGAG GCCGUUAGGC CGAA AGUAUGUC | 1179 |
| 508 | ACAUACUU A AUAUUAAA | 88 | UUUAAUAU CUGAUGAG GCCGUUAGGC CGAA AAGUAUGU | 1180 |
| 511 | UACUUAAU A UUAAAAUU | 89 | AAUUUUAA CUGAUGAG GCCGUUAGGC CGAA AUUAAGUA | 1181 |
| 513 | CUUAAUAU U AAAAUUUC | 90 | GAAAUUUU CUGAUGAG GCCGUUAGGC CGAA AUAUUAAG | 1182 |
| 514 | UUAAUAUU A AAAUUUCC | 91 | GGAAAUUU CUGAUGAG GCCGUUAGGC CGAA AAUAUUAA | 1183 |
| 519 | AUUAAAAU U UCCCAUUU | 92 | AAAUGGGA CUGAUGAG GCCGUUAGGC CGAA AUUUUAAU | 1184 |
| 520 | UUAAAAUU U CCCAUUUU | 93 | AAAAUGGG CUGAUGAG GCCGUUAGGC CGAA AAUUUUAA | 1185 |
| 521 | UAAAAUUU C CCAUUUUU | 94 | AAAAAUGG CUGAUGAG GCCGUUAGGC CGAA AAAUUUUA | 1186 |
| 526 | UUUCCCAU U UUUUAAAA | 95 | UUUUAAAA CUGAUGAG GCCGUUAGGC CGAA AUGGGAAA | 1187 |
| 527 | UUCCCAUU U UUUAAAAA | 96 | UUUUUAAA CUGAUGAG GCCGUUAGGC CGAA AAUGGGAA | 1188 |
| 528 | UCCCAUUU U UUAAAAAU | 97 | AUUUUUAA CUGAUGAG GCCGUUAGGC CGAA AAAUGGGA | 1189 |
| 529 | CCCAUUUU U UAAAAAUG | 98 | CAUUUUUA CUGAUGAG GCCGUUAGGC CGAA AAAAUGGG | 1190 |
| 530 | CCAUUUUU U AAAAAUGG | 99 | CCAUUUUU CUGAUGAG GCCGUUAGGC CGAA AAAAAUGG | 1191 |
| 531 | CAUUUUUU A AAAAUGGA | 100 | UCCAUUUU CUGAUGAG GCCGUUAGGC CGAA AAAAAAUG | 1192 |
| 544 | UGGAGAGU C UGAAUUUU | 101 | AAAAUUCA CUGAUGAG GCCGUUAGGC CGAA ACUCUCCA | 1193 |
| 550 | GUCUGAAU U UUAUUAGA | 102 | UCUAAUAA CUGAUGAG GCCGUUAGGC CGAA AUUCAGAC | 1194 |
| 551 | UCUGAAUU U UAUUAGAG | 103 | CUCUAAUA CUGAUGAG GCCGUUAGGC CGAA AAUUCAGA | 1195 |
| 552 | CUGAAUUU U AUUAGAGC | 104 | GCUCUAAU CUGAUGAG GCCGUUAGGC CGAA AAAUUCAG | 1196 |
| 553 | UGAAUUUU A UUAGAGCU | 105 | AGCUCUAA CUGAUGAG GCCGUUAGGC CGAA AAAAUUCA | 1197 |
| 555 | AAUUUUAU U AGACCUCA | 106 | UGAGCUCU CUGAUGAG GCCGUUAGGC CGAA AUAAAAUU | 1198 |
| 556 | AUUUUAUU A GAGCUCAC | 107 | GUGAGCUC CUGAUGAG GCCGUUAGGC CGAA AAUAAAAU | 1199 |
| 562 | UUAGAGCU C ACACACCA | 108 | UGGUGUGU CUGAUGAG GCCGUUAGGC CGAA AGCUCUAA | 1200 |
| 572 | CACACCAU A UAUUAACA | 109 | UGUUAAUA CUGAUGAG GCCGUUAGGC CGAA AUGGUGUG | 1201 |
| 574 | CACCAUAU A UUAACAUA | 110 | UAUGUUAA CUGAUGAG GCCGUUAGGC CGAA AUAUGGUG | 1202 |
| 576 | CCAUAUAU U AACAUAUA | 111 | UAUAUGUU CUGAUGAG GCCGUUAGGC CGAA AUAUAUGG | 1203 |
| 577 | CAUAUAUU A ACAUAUAC | 112 | GUAUAUGU CUGAUGAG GCCGUUAGGC CGAA AAUAUAUG | 1204 |
| 582 | AUUAACAU A UACAACUG | 113 | CAGUUGUA CUGAUGAG GCCGUUAGGC CGAA AUGUUAAU | 1205 |
| 584 | UAACAUAU A CAACUGUG | 114 | CACAGUUG CUGAUGAG GCCGUUAGGC CGAA AUAUGUUA | 1206 |
| 601 | AACCAGCU A AUCCCUCU | 115 | AGAGGGAU CUGAUGAG GCCGUUAGGC CGAA AGCUGGUU | 1207 |
| 604 | CAGCUAAU C CCUCUGAG | 116 | CUCAGAGG CUGAUGAG GCCGUUAGGC CGAA AUUAGCUG | 1208 |
| 608 | UAAUCCCU C UGAGAAAA | 117 | UUUUCUCA CUGAUGAG GCCGUUAGGC CGAA AGGGAUUA | 1209 |
| 620 | GAAAAACU C CCCAUCUA | 118 | UAGAUGGG CUGAUGAG GCCGUUAGGC CGAA AGUUUUUC | 1210 |
| 626 | CUCCCCAU C UACCCAAU | 119 | AUUGGGUA CUGAUGAG GCCGUUAGGC CGAA AUGGGGAG | 1211 |
| 628 | CCCCAUCU A CCCAAUAC | 120 | GUAUUGGG CUGAUGAG GCCGUUAGGC CGAA AGAUGGGG | 1212 |
| 635 | UACCCAAU A CUGUUACA | 121 | UGUAACAG CUGAUGAG GCCGUUAGGC CGAA AUUGGGUA | 1213 |
| 640 | AAUACUGU U ACAGCAUA | 122 | UAUGCUGU CUGAUGAG GCCGUUAGGC CGAA ACAGUAUU | 1214 |
| 641 | AUACUGUU A CAGCAUAC | 123 | GUAUGCUG CUGAUGAG GCCGUUAGGC CGAA AACAGUAU | 1215 |
| 648 | UACAGCAU A CAAUCUCU | 124 | AGAGAUUG CUGAUGAG GCCGUUAGGC CGAA AUGCUGUA | 1216 |
| 653 | CAUACAAU C UCUGUUCU | 125 | AGAACAGA CUGAUGAG GCCGUUAGGC CGAA AUUGUAUG | 1217 |
| 655 | UACAAUCU C UGUUCUUG | 126 | CAAGAACA CUGAUGAG GCCGUUAGGC CGAA AGAUUGUA | 1218 |
| 659 | AUCUCUGU U CUUGGGCA | 127 | UGCCCAAG CUGAUGAG GCCGUUAGGC CGAA ACAGAGAU | 1219 |
| 660 | UCUCUGUU C UUGGGCAU | 128 | AUGCCCAA CUGAUGAG GCCGUUAGGC CGAA AACAGAGA | 1220 |
| 662 | UCUGUUCU U GGGCAUUU | 129 | AAAUGCCC CUGAUGAG GCCGUUAGGC CGAA AGAACAGA | 1221 |
| 669 | UUGGGCAU U UUGUCAGU | 130 | ACUGACAA CUGAUGAG GCCGUUAGGC CGAA AUGCCCAA | 1222 |
| 670 | UGGGCAUU U UGUCAGUG | 131 | CACUGACA CUGAUGAG GCCGUUAGGC CGAA AAUGCCCA | 1223 |
| 671 | GGGCAUUU U GUCAGUGA | 132 | UCACUGAC CUGAUGAG GCCGUUAGGC CGAA AAAUGCCC | 1224 |
| 674 | CAUUUUGU C AGUGAUGC | 133 | GCAUCACU CUGAUGAG GCCGUUAGGC CGAA ACAAAAUG | 1225 |
| 687 | AUGCUGAU C UUUGCCUU | 134 | AAGGCAAA CUGAUGAG GCCGUUAGGC CGAA AUCAGCAU | 1226 |
| 689 | GCUGAUCU U UGCCUUCU | 135 | AGAAGGCA CUGAUGAG GCCGUUAGGC CGAA AGAUCAGC | 1227 |
| 690 | CUGAUCUU U GCCUUCUU | 136 | AAGAAGGC CUGAUGAG GCCGUUAGGC CGAA AAGAUCAG | 1228 |
| 695 | CUUUGCCU U CUUCCAGG | 137 | CCUGGAAG CUGAUGAG GCCGUUAGGC CGAA AGGCAAAG | 1229 |
| 696 | UUUGCCUU C UUCCAGGA | 138 | UCCUGGAA CUGAUGAG GCCGUUAGGC CGAA AAGGCAAA | 1230 |
| 698 | UGCCUUCU U CCAGGAAC | 139 | GUUCCUGG CUGAUGAG GCCGUUAGGC CGAA AGAAGGCA | 1231 |
| 699 | GCCUUCUU C CAGGAACU | 140 | AGUUCCUG CUGAUGAG GCCGUUAGGC CGAA AAGAAGGC | 1232 |
| 708 | CAGGAACU U GUAAUAGC | 141 | GCUAUUAC CUGAUGAG GCCGUUAGGC CGAA AGUUCCUG | 1233 |
| 711 | GAACUUGU A AUAGCUGG | 142 | CCAGCUAU CUGAUGAG GCCGUUAGGC CGAA ACAAGUUC | 1234 |
| 714 | CUUGUAAU A GCUGGCAU | 143 | AUGCCAGC CUGAUGAG GCCGUUAGGC CGAA AUUACAAG | 1235 |
| 723 | GCUGGCAU C GUUGAGAA | 144 | UUCUCAAC CUGAUGAG GCCGUUAGGC CGAA AUGCCAGC | 1236 |
| 726 | GGCAUCGU U GAGAAUGA | 145 | UCAUUCUC CUGAUGAG GCCGUUAGGC CGAA ACGAUGCC | 1237 |
| 752 | AACGUGCU C CAGACCCA | 146 | UCGGUCUG CUGAUGAG GCCGUUAGGC CGAA AGCACGUU | 1238 |
| 764 | ACCCAAAU C UAACAUAG | 147 | CUAUGUUA CUGAUGAG GCCGUUAGGC CGAA AUUUGGGU | 1239 |
| 766 | CCAAAUCU A ACAUAGUU | 148 | AACUAUGU CUGAUGAG GCCGUUAGGC CGAA AGAUUUGG | 1240 |
| 771 | UCUAACAU A GUUCUCUU | 149 | AGGAGAAC CUGAUGAG GCCGUUAGGC CGAA AUGUUAGA | 1241 |
| 774 | AACAUAGU U CUCCUGUC | 150 | GACAGGAG CUGAUGAG GCCGUUAGGC CGAA ACUAUGUU | 1242 |
| 775 | ACAUAGUU C UCCUGUCA | 151 | UGACAGGA CUGAUGAG GCCGUUAGGC CGAA AACUAUGU | 1243 |
| 777 | AUAGUUCU C CUGUCAGC | 152 | GCUGACAG CUGAUGAG GCCGUUAGGC CGAA AGAACUAU | 1244 |
| 782 | UCUCCUGU C AGCAGAAG | 153 | CUUCUGCU CUGAUGAG GCCGUUAGGC CGAA ACAGGAGA | 1245 |
| 808 | AACAGACU A UUGAAAUA | 154 | UAUUUCAA CUGAUGAG GCCGUUAGGC CGAA AGUCUGUU | 1246 |
| 810 | CAGACUAU U GAAAUAAA | 155 | UUUAUUUC CUGAUGAG GCCGUUAGGC CGAA AUAGUCUG | 1247 |
| 816 | AUUGAAAU A AAAGAAGA | 156 | UCUUCUUU CUGAUGAG GCCGUUAGGC CGAA AUUUCAAU | 1248 |
| 831 | GAAGUGGU U GGGCUAAC | 157 | GUUAGCCC CUGAUGAG GCCGUUAGGC CGAA ACCACUUC | 1249 |
| 837 | GUUGGGCU A ACUGAAAC | 158 | GUUUCAGU CUGAUGAG GCCGUUAGGC CGAA AGCCCAAC | 1250 |
| 848 | UGAAACAU C UUCCCAAC | 159 | GUUGGGAA CUGAUGAG GCCGUUAGGC CGAA AUGUOUCA | 1251 |
| 850 | AAACAUCU U CCCAACCA | 160 | UGGUUGGG CUGAUGAG GCCGUUAGGC CGAA AGAUGUUU | 1252 |
| 851 | AACAUCUU C CCAACCAA | 161 | UUGGUUGG CUGAUGAG GCCGUUAGGC CGAA AAGAUGUU | 1253 |
| 876 | GAAGACAU U GAAAUUAU | 162 | AUAAUUUC CUGAUGAG GCCGUUAGGC CGAA AUGUCUUC | 1254 |
| 882 | AUUGAAAU U AUUCCAAU | 163 | AUUGGAAU CUGAUGAG GCCGUUAGGC CGAA AUUUCAAU | 1255 |
| 883 | UUGAAAUU A UUCCAAUC | 164 | GAUUGGAA CUGAUGAG GCCGUUAGGC CGAA AAUUUCAA | 1256 |
| 885 | GAAAUUAU U CCAAUCCA | 165 | UGGAUUGG CUGAUGAG GCCGUUAGGC CGAA AUAAUUUC | 1257 |
| 886 | AAAUUAUU C CAAUCCAA | 186 | UUGGAUUG CUGAUGAG GCCGUUAGGC CGAA AAUAAUUU | 1258 |
| 891 | AUUCCAAU C CAAGAAGA | 167 | UCUUCUUG CUGAUGAG GCCGUUAGGC CGAA AUUGGAAU | 1259 |
| 926 | GACGAACU U UCCAGAAC | 168 | GUCCUGGA CUGAUGAG GCCGUUAGGC CGAA AGUUCGUC | 1260 |
| 927 | ACGAACUU U CCAGAACC | 169 | GGUUCUGG CUGAUGAG GCCGUUAGGC CGAA AAGUUCGU | 1261 |
| 928 | CGAACUUU C CAGAACCU | 170 | AGGUUCUG CUGAUGAG GCCGUUAGGC CGAA AAAGUUCG | 1262 |
| 937 | CAGAACCU C CCCAAGAU | 171 | AUCUUGGG CUGAUGAG GCCGUUAGGC CGAA AGGUUCUG | 1263 |
| 946 | CCCAAGAU C AGGAAUCC | 172 | GGAUUCCU CUGAUGAG GCCGUUAGGC CGAA AUCUUGGG | 1264 |
| 953 | UCAGGAAU C CUCACCAA | 173 | UUGGUGAG CUGAUGAG GCCGUUAGGC CGAA AUUCCUGA | 1265 |
| 956 | GGAAUCCU C ACCAAUAG | 174 | CUAUUGGU CUGAUGAG GCCGUUAGGC CGAA AGGAUUCC | 1266 |
| 963 | UCACCAAU A GAAAAUGA | 175 | UCAUUUUC CUGAUGAG GCCGUUAGGC CGAA AUUGGUGA | 1267 |
| 977 | UGACAGCU C UCCUUAAG | 176 | CUUAAGGA CUGAUGAG GCCGUUAGGC CGAA AGCUGUCA | 1268 |
| 979 | ACAGCUCU C CUUAAGUG | 177 | CACUUAAG CUGAUGAG GCCGUUAGGC CGAA AGAGCUGU | 1269 |
| 982 | GCUCUCCU U AAGUGAUU | 178 | AAUCACUU CUGAUGAG GCCGUUAGGC CGAA AGGAGAGC | 1270 |
| 983 | CUCUCCUU A AGUGAUUU | 179 | AAAUCACU CUGAUGAG GCCGUUAGGC CGAA AAGGAGAG | 1271 |
| 990 | UAAGUGAU U UCUUCUGU | 180 | ACAGAAGA CUGAUGAG GCCGUUAGGC CGAA AUCACUUA | 1272 |
| 991 | AAGUGAUU U CUUCUGUU | 181 | AACAGAAG CUGAUGAG GCCGUUAGGC CGAA AAUCACUU | 1273 |
| 992 | AGUGAUUU C UUCUGUUU | 182 | AAACAGAA CUGAUGAG GCCGUUAGGC CGAA AAAUCACU | 1274 |
| 994 | UGAUUUCU U CUGUUUUC | 183 | GAAAACAG CUGAUGAG GCCGUUAGGC CGAA AGAAAUCA | 1275 |
| 995 | GAUUUCUU C UGUUUUCU | 184 | AGAAAACA CUGAUGAG GCCGUUAGGC CGAA AAGAAAUC | 1276 |
| 999 | UCUUCUGU U UUCUGUUU | 185 | AAACAGAA CUGAUGAG GCCGUUAGGC CGAA ACAGAAGA | 1277 |
| 1000 | CUUCUGUU U UCUGUUUC | 186 | GAAACAGA CUGAUGAG GCCGUUAGGC CGAA AACAGAAG | 1278 |
| 1001 | UUCUGUUU U CUGUUUCC | 187 | GGAAACAG CUGAUGAG GCCGUUAGGC CGAA AAACAGAA | 1279 |
| 1002 | UCUGUUUU C UGUUUCCU | 188 | AGGAAACA CUGAUGAG GCCGUUAGGC CGAA AAAACAGA | 1280 |
| 1006 | UUUUCUGU U UCCUUUUU | 189 | AAAAAGGA CUGAUGAG GCCGUUAGGC CGAA ACAGAAAA | 1281 |
| 1007 | UUUCUGCU U CCUUUUUU | 190 | AAAAAAGG CUGAUGAG GCCGUUAGGC CGAA AACAGAAA | 1282 |
| 1008 | UUCUGUUU C CUUUUUUA | 191 | UAAAAAAG CUGAUGAG GCCGUUAGGC CGAA AAACAGAA | 1283 |
| 1011 | UGUUUCCU U UUUUAAAC | 192 | GUUUAAAA CUGAUGAG GCCGUUAGGC CGAA AGGAAACA | 1284 |
| 1012 | GUUUCCUU U UUUAAACA | 193 | UGUUUAAA CUGAUGAG GCCGUUAGGC CGAA AAGGAAAC | 1285 |
| 1013 | UUUCCUUU U UUAAACAU | 194 | AUGUUUAA CUGAUGAG GCCGUUAGGC CGAA AAAGGAAA | 1286 |
| 1014 | UUCCUUUU U UAAACAUU | 195 | AAUGUUUA CUGAUGAG GCCGUUAGGC CGAA AAAAGGAA | 1287 |
| 1015 | UCCUUUUU A AAACAUUA | 196 | UAAUGUUU CUGAUGAG GCCGUUAGGC CGAA AAAAAGGA | 1288 |
| 1016 | CCUUUUUU A AACAUUAG | 197 | CUAAUGUU CUGAUGAG GCCGUUAGGC CGAA AAAAAAGG | 1289 |
| 1022 | UUAAACAU U AGUGUUCA | 198 | UGAACACU CUGAUGAG GCCGUUAGGC CGAA AUGUUUAA | 1290 |
| 1023 | UAAACAUU A GUGUUCAU | 199 | AUGAACAC CUGAUGAG GCCGUUAGGC CGAA AAUGUUUA | 1291 |
| 1028 | AUUAGUGU U CAUAGCUU | 200 | AAGCUAUG CUGAUGAG GCCGUUAGGC CGAA ACACUAAU | 1292 |
| 1029 | UUAGUGUU C AUAGCUUC | 201 | CAAGCUAU CUGAUGAG GCCGUUAGGC CGAA AACACUAA | 1293 |
| 1032 | GUGUUCAU A GCUUCCAA | 202 | UUGGAAGC CUGAUGAG GCCGUUAGGC CGAA AUGAACAC | 1294 |
| 1036 | UCAUAGCU U CCAAGAGA | 203 | UCUCUUGG CUGAUGAG GCCGUUAGGC CGAA AGCUAUGA | 1295 |
| 1037 | CAUAGCUU C CAAGAGAC | 204 | GUCUCUUG CUGAUGAG GCCGUUAGGC CGAA AAGCUAUG | 1296 |
| 1055 | UGCUGACU U UCAUUUCU | 205 | AGAAAUGA CUGAUGAG GCCGUUAGGC CGAA AGUCAGCA | 1297 |
| 1056 | GCUGACUU U CAUUUCUU | 206 | AAGAAAUG CUGAUGAG GCCGUUAGGC CGAA AAGUCAGC | 1298 |
| 1057 | CUGACUUU C AUUUCUUG | 207 | CAAGAAAU CUGAUGAG GCCGUUAGGC CGAA AAAGUCAG | 1299 |
| 1060 | ACUUUCAU U UCUUGAGG | 208 | CCUCAAGA CUGAUGAG GCCGUUAGGC CGAA AUGAAAGU | 1300 |
| 1061 | CUUUCAUU U CUUGAGGU | 209 | ACCUCAAG CUGAUGAG GCCGUUAGGC CGAA AAUGAAAG | 1301 |
| 1062 | UUUCAUUU C UUGAGGUA | 210 | UACCUCAA CUGAUGAG GCCGUUAGGC CGAA AAAUGAAA | 1302 |
| 1064 | UCAUUUCU U GAGGUACU | 211 | AGUACCUC CUGAUGAG GCCGUUAGGC CGAA AGAAAUGA | 1303 |
| 1070 | CUUGAGGU A CUCUGCAC | 212 | GUGCAGAG CUGAUGAG GCCGUUAGGC CGAA ACCUCAAG | 1304 |
| 1073 | GAGGUACU C UGCACAUA | 213 | UAUGUGCA CUGAUGAG GCCGUUAGGC CGAA AGUACCUC | 1305 |
| 1081 | CUGCACAU A CGCACCAC | 214 | GUGGUGCG CUGAUGAG GCCGUUAGGC CGAA AUGUGCAG | 1306 |
| 1092 | CACCACAU C UCUAUCUG | 215 | CAGAUAGA CUGAUGAG GCCGUUAGGC CGAA AUGUGGUG | 1307 |
| 1094 | CCACAUCU C UAUCUGGC | 216 | GCCAGAUA CUGAUGAG GCCGUUAGGC CGAA AGAUGUGG | 1308 |
| 1096 | ACAUCUCU A UCUGGCCU | 217 | AGGCCAGA CUGAUGAG GCCGUUAGGC CGAA AGAGAUGU | 1309 |
| 1098 | AUCUCUAU C UGGCCUUU | 218 | AAAGGCCA CUGAUGAG GCCGUUAGGC CGAA AUAGAGAU | 1310 |
| 1105 | UCUGGCCU U UGCAUGGA | 219 | UCCAUGCA CUGAUGAG GCCGUUAGGC CGAA AGGCCAGA | 1311 |
| 1105 | CUGGCCUU U GCAUGGAG | 220 | CUCCAUGC CUGAUGAG GCCGUUAGGC CGAA AAGGCCAG | 1312 |
| 1122 | GUGACCAU A GCUCCUUC | 221 | GAAGGAGC CUGAUGAG GCCGUUAGGC CGAA AUGGUCAC | 1313 |
| 1126 | CCAUAGCU C CUUCUCUC | 222 | GAGAGAAG CUGAUGAG GCCGUUAGGC CGAA AGCUAUGG | 1314 |
| 1129 | UAGCUCCU U CUCUCUUA | 223 | UAAGAGAG CUGAUGAG GCCGUUAGGC CGAA AGGAGCUA | 1315 |
| 1130 | AGCUCCUU C UCUCUUAC | 224 | GUAAGAGA CUGAUGAG GCCGUUAGGC CGAA AAGGAGCU | 1316 |
| 1132 | CUCCUUCU C UCUUACAU | 225 | AUGUAAGA CUGAUGAG GCCGUUAGGC CGAA AGAUGGAG | 1317 |
| 1134 | CCUUCUCU C UUACAUUG | 226 | CAAUGUAA CUGAUGAG GCCGUUAGGC CGAA AGAGAAGG | 1318 |
| 1136 | UUCUCUCU U ACAUUGAA | 227 | UUCAAUGU CUGAUGAG GCCGUUAGGC CGAA AGAGAGAA | 1319 |
| 1137 | UCUCUCUU A CAUUGAAU | 228 | AUUCAAUG CUGAUGAG GCCGUUAGGC CGAA AAGAGAGA | 1320 |
| 1141 | UCUUACAU U GAAUGUAG | 229 | CUACAUUC CUGAUGAG GCCGUUAGGC CGAA AUGUAAGA | 1321 |
| 1148 | UUGAAUGU A GAGAAUGU | 230 | ACAUUCUC CUGAUGAG GCCGUUAGGC CGAA ACAUUCAA | 1322 |
| 1157 | GAGAAUGU A GCCAUUGU | 231 | ACAAUGGC CUGAUGAG GCCGUUAGGC CGAA ACAUUCUC | 1323 |
| 1163 | GUAGCCAU U GUAGCAGC | 232 | GCUGCUAC CUGAUGAG GCCGUUAGGC CGAA AUGGCUAC | 1324 |
| 1166 | GCCAUUGU A GCAGCUUG | 233 | CAAGCUGC CUGAUGAG GCCGUUAGGC CGAA ACAAUGGC | 1325 |
| 1173 | UAGCAGCU U GUGUUGUC | 234 | GACAACAC CUGAUGAG GCCGUUAGGC CGAA AGCUGCUA | 1326 |
| 1178 | GCUUGUGU U GUCACGCU | 235 | AGCGUGAC CUGAUGAG GCCGUUAGGC CGAA ACACAAGC | 1327 |
| 1181 | UGUGUUGU C ACGCUUGU | 236 | AGAAGCGU CUGAUGAG GCCGUUAGGC CGAA ACAACACA | 1328 |
| 1187 | GUCACGCU U CUUCUUUU | 237 | AAAAGAAG CUGAUGAG GCCGUUAGGC CGAA AGCGUGAC | 1329 |
| 1188 | UCACGCUU C UUCUUUUG | 238 | CAAAAGAA CUGAUGAG GCCGUUAGGC CGAA AAGCGUGA | 1330 |
| 1190 | ACGCUUCU U CUUUUGAG | 239 | CUCAAAAG CUGAUGAG GCCGUUAGGC CGAA AGAAGCGU | 1331 |
| 1191 | CGCUUCUU C UUUUGAGC | 240 | GCUCAAAA CUGAUGAG GCCGUUAGGC CGAA AAGAAGCG | 1332 |
| 1193 | CUUCUUCU U UUGAGCAA | 241 | UUGCUCAA CUGAUGAG GCCGUUAGGC CGAA AGAAGAAG | 1333 |
| 1194 | UUCUUCUU U UGAGCAAC | 242 | GUUUCUCA CUGAUGAG GCCGUUAGGC CGAA AAGAAGAA | 1334 |
| 1195 | UCUUCUUU U GAGCAACU | 243 | AGUUGCUC CUGAUGAG GCCGUUAGGC CGAA AAAGAAGA | 1335 |
| 1204 | GAGCAACU U UCUUACAC | 244 | GUGUAAGA CUGAUGAG GCCGUUAGGC CGAA AGUUGCUC | 1336 |
| 1205 | AGCAACUU U CUUACACU | 245 | AGUGUAAG CUGAUGAG GCCGUUAGGC CGAA AAGUUGCU | 1337 |
| 1206 | GCAACUUU C UUACACUG | 246 | CAGUGUAA CUGAUGAG GCCGUUAGGC CGAA AAAGUUGC | 1338 |
| 1208 | AACUUUCU U ACACUGAA | 247 | UUCAGUGU CUGAUGAG GCCGUUAGGC CGAA AGAAAGUU | 1339 |
| 1209 | ACUUUUUU A CACUGAAG | 248 | CUUCAGUG CUGAUGAG GCCGUUAGGC CGAA AAGAAAGU | 1340 |
| 1236 | UGAGUGCU U CAGAAUGU | 249 | ACAUUCUG CUGAUGAG GCCGUUAGGC CGAA AGCACUCA | 1341 |
| 1237 | GAGUGCUU C AGAAUGUG | 250 | CACAUUCU CUGAUGAG GCCGUUAGGC CGAA AAGCACUC | 1342 |
| 1248 | AAUGUGAU U UCCUACUA | 251 | UAGUAGGA CUGAUGAG GCCGUUAGGC CGAA AUCACAUU | 1343 |
| 1249 | AUGUGAUU U CCUACUAA | 252 | UUAGUAGG CUGAUGAG GCCGUUAGGC CGAA AAUCACAU | 1344 |
| 1250 | UGUGAUUU C CUACUAAC | 253 | GUUAGUAG CUGAUGAG GCCGUUAGGC CGAA AAAUCACA | 1345 |
| 1253 | GAUUUCCU A CUAACCUG | 254 | CAGGUUAG CUGAUGAG GCCGUUAGGC CGAA AGGAAAUC | 1346 |
| 1256 | UUCCUACU A ACCUGUUC | 255 | GAACAGGU CUGAUGAG GCCGUUAGGC CGAA AGUAGGAA | 1347 |
| 1263 | UAACCUGU U CCUUGGAU | 256 | AUCCAAGG CUGAUGAG GCCGUUAGGC CGAA ACAGGUUA | 1348 |
| 1264 | AACCUGUU C CUUGGAUA | 257 | UAUCCAAG CUGAUGAG GCCGUUAGGC CGAA AACAGGUU | 1349 |
| 1267 | CUGUUCCU U GGAUAGGC | 258 | GCCUAUCC CUGAUGAG GCCGUUAGGC CGAA AGGAACAG | 1350 |
| 1272 | CCUUGGAU A GGCUUUUU | 259 | AAAAAGCC CUGAUGAG GCCGUUAGGC CGAA AUCCAAGG | 1351 |
| 1277 | GAUAGGCU U UUUAGUAU | 260 | AUACUAAA CUGAUGAG GCCGUUAGGC CGAA AGCCUAUC | 1352 |
| 1278 | AUAGGCUU U UUAGUAUA | 261 | UAUACUAA CUGAUGAG GCCGUUAGGC CGAA AAGCCUAU | 1353 |
| 1279 | UAGGCUUU U UAGUAUAG | 262 | CUAUACUA CUGAUGAG GCCGUUAGGC CGAA AAAGCCUA | 1354 |
| 1280 | AGGCUUUU U AGUAUAGU | 263 | ACUAUACU CUGAUGAG GCCGUUAGGC CGAA AAAAGCCU | 1355 |
| 1281 | GGCUUUUU A GUAUAGUA | 264 | UACUAUAC CUGAUGAG GCCGUUAGGC CGAA AAAAAGCC | 1356 |
| 1284 | UUUUUAGU A UAGUAUUU | 265 | AAAUACUA CUGAUGAG GCCGUUAGGC CGAA ACUAAAAA | 1357 |
| 1286 | UUUAGUAU A GUAUUUUU | 266 | AAAAAUAC CUGAUGAG GCCGUUAGGC CGAA AUACUAAA | 1358 |
| 1289 | AGUAGUAU A UUUUUUU | 267 | AAAAAAAA CUGAUGAG GCCGUUAGGC CGAA ACUAUACU | 1359 |
| 1291 | UAUAUUAU U UUUUUUUG | 268 | CAAAAAAA CUGAUGAG GCCGUUAGGC CGAA AUACUAUA | 1360 |
| 1292 | AUAGUAUU U UUUUUUGU | 269 | ACAAAAAA CUGAUGAG GCCGUUAGGC CGAA AAUACUAU | 1361 |
| 1293 | UAGUAUUU U UUUUUGUC | 270 | GACAAAAA CUGAUGAG GCCGUUAGGC CGAA AAAUACUA | 1362 |
| 1294 | AGUAUUUU U UUUUGUCA | 271 | UGACAAAA CUGAUGAG GCCGUUAGGC CGAA AAAAUACU | 1363 |
| 1295 | GUAUUUUU U UUUGUCAU | 272 | AUGACAAA CUGAUGAG GCCGUUAGGC CGAA AAAAAUAC | 1364 |
| 1296 | UAUUUUUU U UUGUCAUU | 273 | AAUGACAA CUGAUGAG GCCGUUAGGC CGAA AAAAAAUA | 1365 |
| 1297 | AUUUUUUU U UGUCAUUU | 274 | AAAUGACA CUGAUGAG GCCGUUAGGC CGAA AAAAAAAU | 1366 |
| 1298 | UUUUUUUU U GUCAUUUU | 275 | AAAAUGAC CUGAUGAG GCCGUUAGGC CGAA AAAAAAAA | 1367 |
| 1301 | UUUUUUGU C AUUUUCUC | 276 | GAGAAAAU CUGAUGAG GCCGUUAGGC CGAA ACAAAAAA | 1368 |
| 1304 | UUUGUCAU U UUCUCCAU | 277 | AUGGAGAA CUGAUGAG GCCGUUAGGC CGAA AUGACAAA | 1369 |
| 1305 | UUGUCAUU U UCUCCAUC | 278 | GAUGGAGA CUGAUGAG GCCGUUAGGC CGAA AAUGACAA | 1370 |
| 1306 | UGUCAUUU U CUCCAUCA | 279 | UGAUGGAG CUGAUGAG GCCGUUAGGC CGAA AAAUGACA | 1371 |
| 1307 | GUCAUUUU C UCCAUCAG | 280 | CUGAUGGA CUGAUGAG GCCGUUAGGC CGAA AAAAUGAC | 1372 |
| 1309 | CAUUUUCU C CAUCAGCA | 281 | UGCUGAUG CUGAUGAG GCCGUUAGGC CGAA AGAAAAUG | 1373 |
| 1313 | UUCUCCAU C AGCAACCA | 282 | UGGUUGCU CUGAUGAG GCCGUUAGGC CGAA AUGGAGAA | 1374 |
| 1348 | GAAAAGAU A UAUGACUG | 283 | CAGUCAUA CUGAUGAG GCCGUUAGGC CGAA AUCUUUUC | 1375 |
| 1350 | AAAGAUAU A UGACUGCU | 284 | AGCAGUCA CUGAUGAG GCCGUUAGGC CGAA AUAUCUUU | 1376 |
| 1359 | UGACUGCU U CAUGACAU | 285 | AUGUCAUG CUGAUGAG GCCGUUAGGC CGAA AGCAGUCA | 1377 |
| 1360 | GACUGCUU C AUGACAUU | 286 | AAUGUCAU CUGAUGAG GCCGUUAGGC CGAA AAGCAGUC | 1378 |
| 1368 | CAUGACAU U CCUAAACU | 287 | AGUUUAGG CUGAUGAG GCCGUUAGGC CGAA AUGUCAUG | 1379 |
| 1369 | AUGACAUU C CUAAACUA | 288 | UAGUUUAG CUGAUGAG GCCGUUAGGC CGAA AAUGUCAU | 1380 |
| 1372 | ACAUUCCU A AACUAUCU | 289 | AGAUAGUU CUGAUGAG GCCGUUAGGC CGAA AGGAAUGU | 1381 |
| 1377 | CCUAAACU A UCUUUUUU | 290 | AAAAAAGA CUGAUGAG GCCGUUAGGC CGAA AGUUUAGG | 1382 |
| 1379 | UAAACUAU C UUUUUUUU | 291 | AAAAAAAA CUGAUGAG GCCGUUAGGC CGAA AUAGUUUA | 1383 |
| 1381 | AACUAUCU U UUUUUUAU | 292 | AUAAAAAA CUGAUGAG GCCGUUAGGC CGAA AGAUAGUU | 1384 |
| 1382 | ACUAUCUU U UUUUUAUU | 293 | AAUAAAAA CUGAUGAG GCCGUUAGGC CGAA AAGAUAGU | 1385 |
| 1383 | CUAUCUUU U UUUUAUUC | 294 | GAAUAAAA CUGAUGAG GCCGUUAGGC CGAA AAAGAUAG | 1386 |
| 1384 | UAUCUUUU U UUUAUUCC | 295 | GGAAUAAA CUGAUGAG GCCGUUAGGC CGAA AAAAGAUA | 1387 |
| 1385 | AUCUUUUU U UUAUUCCA | 296 | UGGAAUAA CUGAUGAG GCCGUUAGGC CGAA AAAAAGAU | 1388 |
| 1386 | UCUUUUUU U UAUUCCAC | 297 | GUGGAAUA CUGAUGAG GCCGUUAGGC CGAA AAAAAAGA | 1389 |
| 1387 | UCUUUUUU U AUUCCACA | 298 | UGUGGAAU CUGAUGAG GCCGUUAGGC CGAA AAAAAAAG | 1390 |
| 1388 | UUUUUUUU A UUCCACAU | 299 | AUGUGGAA CUGAUGAG GCCGUUAGGC CGAA AAAAAAAA | 1391 |
| 1390 | UUUUUUAU U CCACAUCU | 300 | AGAUGUGG CUGAUGAG GCCGUUAGGC CGAA AUAAAAAA | 1392 |
| 1391 | UUUUUAUU C CACAUCUA | 301 | UAGAUGUG CUGAUGAG GCCGUUAGGC CGAA AAUAAAAA | 1393 |
| 1397 | UUCCACAU C UACGUUUU | 302 | AAAACGUA CUGAUGAG GCCGUUAGGC CGAA AUGUGGAA | 1394 |
| 1399 | CCACAUUU A CGUUUUUG | 303 | CAAAAACG CUGAUGAG GCCGUUAGGC CGAA AGAUGUGG | 1395 |
| 1403 | AUCUACGU U UUUGGUGG | 304 | CCACCAAA CUGAUGAG GCCGUUAGGC CGAA ACGUAGAU | 1396 |
| 1404 | UCUACGUU U UUGGUGGA | 305 | UCCACCAA CUGAUGAG GCCGUUAGGC CGAA AACGUAGA | 1397 |
| 1405 | CUACGUUU U UGGUGGAG | 306 | CUCCACCA CUGAUGAG GCCGUUAGGC CGAA AAACGUAG | 1398 |
| 1406 | UACGUUUU U UGUGGAGU | 307 | ACUCCACC CUGAUGAG GCCGUUAGGC CGAA AAAACGUA | 1399 |
| 1415 | GGUGGAGU C CCUUUUGC | 308 | GCAAAAGG CUGAUGAG GCCGUUAGGC CGAA ACUCCACC | 1400 |
| 1419 | GAGUCCCU U UUGCAUCA | 309 | UGAUGCAA CUGAUGAG GCCGUUAGGC CGAA AGGGACUC | 1401 |
| 1420 | AGUOCCUU U UGCAUCAU | 310 | AUGAUGCA CUGAUGAG GCCGUUAGGC CGAA AAGGGACU | 1402 |
| 1421 | GUCCCUUU U GCAUCAUU | 311 | AAUGAUGC CUGAUGAG GCCGUUAGGC CGAA AAAGGGAC | 1403 |
| 1426 | UUUUGCAU C AUUGUUUU | 312 | AAAACAAU CUGAUGAG GCCGUUAGGC CGAA AUGCAAAA | 1404 |
| 1429 | UGCAUCAU U GUUUUAAG | 313 | CUUAAAAC CUGAUGAG GCCGUUAGGC CGAA AUGAUGCA | 1405 |
| 1432 | AUCAUUGU U UUAAGGAU | 314 | AUCCUUAA CUGAUGAG GCCGUUAGGC CGAA ACAAUGAU | 1406 |
| 1433 | UCAUUGUU U UAAGGAUG | 315 | CAUCCUUA CUGAUGAG GCCGUUAGGC CGAA AACAAUGA | 1407 |
| 1434 | CAUUGUUU U AAGGAUGA | 316 | UCAUCCUU CUGAUGAG GCCGUUAGGC CGAA AAACAAUG | 1408 |
| 1435 | AUUGUUUU A AGGAUGAU | 317 | AUCAUCCU CUGAUGAG GCCGUUAGGC CGAA AAAACAAU | 1409 |
| 1444 | AGGAUGAU A AAAAAAAA | 318 | UUUUUUUU CUGAUGAG GCCGUUAGGC CGAA AUCAUCCU | 1410 |
| 1455 | AAAAAAAU A ACAACUAG | 319 | CUAGUUGU CUGAUGAG GCCGUUAGGC CGAA AUUUUUUU | 1411 |
| 1462 | UAACAACU A GGGACAAU | 320 | AUUGUCCC CUGAUGAG GCCGUUAGGC CGAA AGUUGUUA | 1412 |
| 1471 | GGGACAAU A CAGAACCC | 321 | UGGUUCUG CUGAUGAG GCCGUUAGGC CGAA AUUGUCCC | 1413 |
| 1482 | GAACCCAU U CCAUUUAU | 322 | AUAAAUGG CUGAUGAG GCCGUUAGGC CGAA AUGGGUUC | 1414 |
| 1483 | AACCCAUU C CAUUUAUC | 323 | GAUAAAUG CUGAUGAG GCCGUUAGGC CGAA AAUGGGUU | 1415 |
| 1487 | CAUUCCAU U UAUCUUUC | 324 | GAAAGAUA CUGAUGAG GCCGUUAGGC CGAA AUGGAAUG | 1416 |
| 1488 | AUUCCAUU U AUCUUUCU | 325 | AGAAAGAU CUGAUGAG GCCGUUAGGC CGAA AAUGGAAU | 1417 |
| 1489 | UUCCAUUU A UCUUUCUA | 326 | UAGAAAGA CUGAUGAG GCCGUUAGGC CGAA AAAUGGAA | 1418 |
| 1491 | CCAUUUAU C UUUCUACA | 327 | UGUAGAAA CUGAUGAG GCCGUUAGGC CGAA AUAAAUGG | 1419 |
| 1493 | AUUUAUCU U UCUACAGG | 328 | CCUGUAGA CUGAUGAG GCCGUUAGGC CGAA AGAUAAAU | 1420 |
| 1494 | UUUAUCUU U CUACAGGG | 329 | CCCUGUAG CUGAUGAG GCCGUUAGGC CGAA AAGAUAAA | 1421 |
| 1495 | UUAUCUUU C UACAGGGC | 330 | GCCCUGUA CUGAUGAG GCCGUUAGGC CGAA AAAGAUAA | 1422 |
| 1497 | AUCUUUCU A CAGGGCUG | 331 | CAGCCCUG CUGAUGAG GCCGUUAGGC CGAA AGAAAGAU | 1423 |
| 1510 | GCUGACAU U GUGGCACA | 332 | UGUGCCAC CUGAUGAG GCCGUUAGGC CGAA AUGUCAGC | 1424 |
| 1520 | UGGCACAU U CUUAGAGU | 333 | ACUCUAAG CUGAUGAG GCCGUUAGGC CGAA AUGUGCCA | 1425 |
| 1521 | GGCACAUU C UUAGAGUU | 334 | AACUCUAA CUGAUGAG GCCGUUAGGC CGAP AAUGUGCC | 1426 |
| 1523 | CACAUUCU U AGAGUUAC | 335 | GUAACUCU CUGAUGAG GCCGUUAGGC CGAA AGAAUGUG | 1427 |
| 1524 | ACAUUCUU A GAGUUACC | 336 | GGUAACUC CUGAUGAG GCCGUUAGGC CGAA AAGAAUGU | 1428 |
| 1529 | CUUAGAGU U ACCACACC | 337 | GGUGUGGU CUGAUGAG GCCGUUAGGC CGAA ACUCUAAG | 1429 |
| 1530 | UUAGAGUU A CCACACCC | 338 | GGGUGUGG CUGAUGAG GCCGUUAGGC CGAA AACUCUAA | 1430 |
| 1552 | GGGAAGCU C UAAAUAGC | 339 | GCUAUUUA CUGAUGAG GCCGUUAGGC CGAA AGCUUCCC | 1431 |
| 1554 | GAAGCUCU A AAUAGCCA | 340 | UGGCUAUU CUGAUGAG GCCGUUAGGC CGPA AGAGCUUC | 1432 |
| 1558 | CUCUAAAU A GCCAACAC | 341 | GUGUUGGC CUGAUGAG GCCGUUAGGC CGAA AUQUAGAG | 1433 |
| 1571 | ACACCCAU C UGUUUUUU | 342 | AAAAAACA CUGAUGAG GCCGUUAGGC CGAA AUGGGUGU | 1434 |
| 1575 | CCAUCUGU U UUUUGUAA | 343 | UUACAAAA CUGAUGAG GCCGUUAGGC CGAA ACAGAUGG | 1435 |
| 1576 | CAUCUGUU U UUUGUAAA | 344 | UUUACAAA CUGAUGAG GCCGUUAGGC CGAA AACAGAUG | 1436 |
| 1577 | AUCUGUUU U UUGUAAAA | 345 | UUUUACAA CUGAUGAG GCCGUUAGGC CGAA AAACAGAU | 1437 |
| 1578 | UCUGUUUU U UGUAAAAA | 346 | UUUUUACA CUGAUGAG GCCGUUAGGC CGAA AAAACAGA | 1438 |
| 1579 | UCUGUUUU U GUAAAAAC | 347 | GUUUUUAC CUGAUGAG GCCGUUAGGC CGAA AAAAACAG | 1439 |
| 1582 | UUUUUUGU A AAAACAGC | 348 | GCUGUUUU CUGAUGAG GCCGUUAGGC CGAA ACAAAAAA | 1440 |

InputSequence = HSCD20A. Cut Site = UH/.
Stem Length = 8. Core Sequence = CUGAUGAG GCCGUUAGGCCGAA
HSCD20A (Human mRNA for CD20 receptor (87); 1597 bp)

Underlined region can be any X sequence or linker, as previously defined herein.

TABLE IV
Human CD20 Inozyme Ribozyme and Substrate Sequence

| | | | | Rz Seq |
|---|---|---|---|---|
| Pos | Substrate | Seq ID | Ribozyme | ID |
| 11 | CAAACUGC A CCCACUGA | 349 | UCAGUGGG CUGAUGAG GCCGUUAGGC CGAA ICAGUUUG | 1441 |
| 13 | AACUGCAC C CACUGAAC | 350 | GUUCAGUG CUGAUGAG GCCGUUAGGC CGAA IUGCAGUU | 1442 |
| 14 | ACUGCACC C ACUGAACU | 351 | AGUUCAGU CUGAUGAG GCCGUUAGGC CGAA IGUGCAGU | 1443 |
| 15 | CUGCACCC A CUGAACUC | 352 | GAGUUCAG CUGAUGAG GCCGUUAGGC CGAA IGGUCCAG | 1444 |
| 17 | GCACCCAC U GAACUCCG | 353 | CGGAGUUC CUGAUGAG GCCGUUAGGC CGAA IUGGGUGC | 1445 |
| 22 | CACUGAAC U CCGCAGCU | 354 | AGCUGCGG CUGAUGAG GCCGUUAGGC CGAA IUUCAGUG | 1446 |
| 24 | CUGAACUC C GCAGCUAG | 355 | CUAGCUGC CUGAUGAG GCCGUUAGGC CGAA IAGUUCAG | 1447 |
| 27 | AACUCCGC A GCUAGCAU | 356 | AUGCUAGC CUGAUGAG GCCGUUAGGC CGAA ICGGAGUU | 1448 |
| 30 | UCCGCAGC U AGCAUCCA | 357 | UGGAUGCU CUGAUGAG GCCGUUAGGC CGAA ICUGCGGA | 1449 |
| 34 | CAGCUAGC A UCCAAAUC | 358 | GAUUUGGA CUGAUGAG GCCGUUAGGC CGAA ICUAGCUG | 1450 |
| 37 | CUACCAUC C AAAUCAGC | 359 | GCUGAUUU CUGAUGAG GCCGUUAGGC CGAA IAUGCUAG | 1451 |
| 38 | UAGCAUCC A AAUCAGCC | 360 | GGCUGAUU CUGAUGAG GCCGUUAGGC CGAA IGAUGCUA | 1452 |
| 43 | UCCAAAUC A GCCCUUGA | 361 | UCAAGGGC CUGAUGAG GCCGUUAGGC CGAA IAUUUGGA | 1453 |
| 46 | AAAUCAGC C CUUGAGAU | 362 | AUCUCAAG CUGAUGAG GCCGUUAGGC CGAA ICUGAUUU | 1454 |
| 47 | AAUCAGCC C UUGAGAUU | 363 | AAUCUCAA CUGAUGAG GCCGUUAGGC CGAA IGCUGAUU | 1455 |
| 48 | AUCAGCCC U UGAGAUUU | 364 | AAAUCUCA CUGAUGAG GCCGUUAGGC CGAA IGGCUGAU | 1456 |
| 62 | UUUGAGGC C UUGGAGAC | 365 | GUCUCCAA CUGAUGAG GCCGUUAGGC CGAA ICCUCAAA | 1457 |
| 63 | UUGAGGCC U UGGAGACU | 366 | AGUCUCCA CUGAUGAG GCCGUUAGGC CGAA IGCCUCAA | 1458 |
| 71 | UUGGAGAC U CAGGAGUU | 367 | AACUCCUG CUGAUGAG GCCGUUAGGC CGAA IUCUCCAA | 1459 |
| 73 | GGAGACUC A GGAGUUUU | 368 | AAAACUCC CUGAUGAG GCCGUUAGGC CGAA IAGUCUCC | 1460 |
| 88 | UUGAGAGC A AAAUGACA | 369 | UGUCAUUU CUGAUGAG GCCGUUAGGC CGAA ICUCUCAA | 1461 |
| 96 | AAAAUCAC A ACACCCAG | 370 | CUGGGUGU CUGAUGAG GCCGUUAGGC CGAA IUCAUUUU | 1462 |
| 99 | AUGACAAC A CCCAGAAA | 371 | UUUCUGGG CUGAUGAG GCCGUUAGGC CGAA IUUGUCAU | 1463 |
| 101 | GACAACAC C CAGAAAUU | 372 | AAUUUCUG CUGAUGAG GCCGUUAGGC CGAA IUGUUGUC | 1464 |
| 102 | ACAACACC C AGAAAUUC | 373 | GAAUUUCU CUGAUGAG GCCGUUAGGC CGAA IGUGUUGU | 1465 |
| 103 | CAACACCC A GAAAUUCA | 374 | UGAAUUUC CUGAUGAG GCCGUUAGGC CGAA IGGUGUUG | 1466 |
| 111 | AGAAAUUC A GUAAAUGG | 375 | CCAUUUAC CUGAUGAG GCCGUUAGGC CGAA IAAUUUCU | 1467 |
| 123 | AAUGGGAC U UUCCUGGC | 376 | GCCAGGAA CUGAUGAG GCCGUUAGGC CGAA IUCCCAUU | 1468 |
| 127 | GGACUUUC C UGGCAGAG | 377 | CUCUGCCA CUGAUGAG GCCGUUAGGC CGAA IAAAGUCC | 1469 |
| 128 | GACUUUCC U GGCAGAGC | 378 | GCUCUGCC CUGAUGAG GCCGUUAGGC CGAA IGAAAGUC | 1470 |
| 132 | UUCCUGGC A GAGCCAAU | 379 | AUUGGCUC CUGAUGAG GCCGUUAGGC CGAA ICCAGGAA | 1471 |
| 137 | GGCAGAGC C AAUGAAAG | 380 | CUUUCAUU CUGAUGAG GCCGUUAGGC CGAA ICUCUGCC | 1472 |
| 138 | GCAGAGCC A AUGAAAGG | 381 | CCUUUCAU CUGAUGAG GCCGUUAGGC CGAA IGCUCUGC | 1473 |
| 148 | UGAAAGGC C CUAUUGCU | 382 | AGCAAUAG CUGAUGAG GCCGUUAGGC CGAA ICCUUUCA | 1474 |
| 149 | GAAAGGCC C UAUUGCUA | 383 | UAGCAAUA CUGAUGAG GCCGUUAGGC CGAA IGCCUUUC | 1475 |
| 150 | AAAGGCCC U AUUGCUAU | 384 | AUAGCAAU CUGAUGAG GCCGUUAGGC CGAA IGGCCUUU | 1476 |
| 156 | CCUAUUGC U AUGCAAUC | 385 | GAUUGCAU CUGAUGAG GCCGUUAGGC CGAA ICAAUAGG | 1477 |
| 161 | UGCUAUGC A AUCUGGUC | 386 | GACCAGAU CUGAUGAG GCCGUUAGGC CGAA ICAUAGCA | 1478 |
| 165 | AUGCAAUC U GGUCCAAA | 387 | UUUGGACC CUGAUGAG GCCGUUAGGC CGAA IAUUGCAU | 1479 |
| 170 | AUCUGGUC C AAAACCAC | 388 | GUGGUUUU CUGAUGAG GCCGUUAGGC CGAA IACCAGAU | 1480 |
| 171 | UCUGGUCC A AAACCACU | 389 | AGUGGUUU CUGAUGAG GCCGUUAGGC CGAA IGACCAGA | 1481 |
| 176 | UCCAAAAC C ACUCUUCA | 390 | UGAAGAGU CUGAUGAG GCCGUUAGGC CGAA IUUUUGGA | 1482 |
| 177 | CCAAAACC A CUCUUCAG | 391 | CUGAAGAG CUGAUGAG GCCGUUAGGC CGAA IGUUUUGG | 1483 |
| 179 | AAAACCAC U CUUCAGGA | 392 | UCCUGAAG CUGAUGAG GCCGUUAGGC CGAA IUGGUUUU | 1484 |
| 181 | AACCACUC U UCAGGAGG | 393 | CCUCCUGA CUGAUGAG GCCGUUAGGC CGAA IAGUGGUU | 1485 |
| 184 | CACUCUUC A GGAGGAUG | 394 | CAUCCUCC CUGAUGAG GCCGUUAGGC CGAA IAAGAGUG | 1486 |
| 195 | AGGAUGUC U UCACUGGU | 395 | ACCAGUGA CUGAUGAG GCCGUUAGGC CGAA IACAUCCU | 1487 |
| 198 | AUGUCUUC A CUGGUGGG | 396 | CCCACCAG CUGAUGAG GCCGUUAGGC CGAA IAAGACAU | 1488 |
| 200 | GUCUUCAC U GGUGGGCC | 397 | GGCCCACC CUGAUGAG GCCGUUAGGC CGAA IUGAAGAC | 1489 |
| 208 | UGGUGGGC C CCACGCAA | 398 | UUGCGUGG CUGAUGAG GCCGUUAGGC CGAA ICCCACCA | 1490 |
| 209 | GGUGGGCC C CACGCAAA | 399 | UUUGCGUG CUGAUGAG GCCGUUAGGC CGAA IGCCCACC | 1491 |
| 210 | GUGGGCCC C ACGCAAAG | 400 | CUUUGCGU CUGAUGAG GCCGUUAGGC CGAA IGGCCCAC | 1492 |
| 211 | UGGGCCCC A CGCAAAGC | 401 | GCUUUGCG CUGAUGAG GCCGUUAGGC CGAA IGGGCCCA | 1493 |
| 215 | CCCCACGC A AAGCUUCU | 402 | AGAAGCUU CUGAUGAG GCCGUUAGGC CGAA ICGUGGGG | 1494 |
| 220 | CGCAAAGC U UCUUCAUG | 403 | CAUGAAGA CUGAUGAG GCCGUUAGGC CGAA ICUUUGCG | 1495 |
| 223 | AAAGCUUC U UCAUGAGG | 404 | CCUCAUGA CUGAUGAG GCCGUUAGGC CGAA IAAGCUUU | 1496 |
| 226 | GCUUCUUC A UGAGGGAA | 405 | UUCCCUCA CUGAUGAG GCCGUUAGGC CGAA IAAGAAGC | 1497 |
| 237 | AGGGAAUC U AAGACUUU | 406 | AAAGUCUU CUGAUGAG GCCGUUAGGC CGAA IAUUCCCU | 1498 |
| 243 | UCUAAGAC U UUGGGGGC | 407 | GCCCCCAA CUGAUGAG GCCGUUAGGC CGAA IUCUGAGA | 1499 |
| 252 | UUGGGGGC U GUCCAGAU | 408 | AUCUGGAC CUGAUGAG GCCGUUAGGC CGAA ICCCCCAA | 1500 |
| 256 | GGGCUGUC C AGAUUAUG | 409 | CAUAAUCU CUGAUGAG GCCGUUAGGC CGAA IACAGCCC | 1501 |
| 257 | GGCUGUCC A GAUUAUGA | 410 | UCAUAAUC CUGAUGAG GCCGUUAGGC CGAA IGACAGCC | 1502 |
| 272 | GAAUGGGC U CUUCCACA | 411 | UGUGGAAG CUGAUGAG GCCGUUAGGC CGAA ICCCAUUC | 1503 |
| 274 | AUGGGCUC U UCCACAUG | 412 | AAUGUGGA CUGAUGAG GCCGUUAGGC CGAA IAGCCCAU | 1504 |
| 277 | GGCUCUUC C ACAUUGCC | 413 | GGCAAUGU CUGAUGAG GCCGUUAGGC CGAA IAAGAGCC | 1505 |
| 278 | GCUCUUCC A CAUUGCCC | 414 | GGGCAAUG CUGAUGAG GCCGUUAGGC CGAA IGAAGAGC | 1506 |
| 280 | UCUUCCAC A UUGCCCUG | 415 | CAGGGCAA CUGAUGAG GCCGUUAGGC CGAA IUGGAAGA | 1507 |
| 285 | CACAUUGC C CUGGGGGG | 416 | CCCCCCAG CUGAUGAG GCCGUUAGGC CGAA ICAAUGUG | 1508 |
| 286 | ACAUUGCC C UGGGGGGU | 417 | ACCCCCCA CUGAUGAG GCCGUUAGGC CGAA IGCAAUGU | 1509 |
| 287 | CAUUGCCC U GGGGGGUC | 418 | GACCCCCC CUGAUGAG GCCGUUAGGC CGAA IGGCAAUG | 1510 |
| 296 | GGGGGGUC U UCUGAUGA | 419 | UCAUCAGA CUGAUGAG GCCGUUAGGC CGAA IACCCCCC | 1511 |
| 299 | GGGUCUUC U GAUGAUCC | 420 | GGAUCAUC CUGAUGAG GCCGUUAGGC CGAA IAAGACCC | 1512 |
| 307 | UGAUGAUC C CAGCAGGG | 421 | CCCUGCUG CUGAUGAG GCCGUUAGGC CGAA IAUCAUCA | 1513 |
| 308 | GAUGAUCC C AGCAGGGA | 422 | UCCCUGCU CUGAUGAG GCCGUUAGGC CGAA IGAUCAUC | 1514 |
| 309 | AUGAUCCC A GCAGGGAU | 423 | AUCCCUGC CUGAUGAG GCCGUUAGGC CGAA IGGAUCAU | 1515 |
| 312 | AUCCCAGC A GGGAUCUA | 424 | UAGAUCCC CUGAUGAG GCCGUUAGGC CGAA ICUGGGAU | 1516 |
| 319 | CAGGGAUC U AUGCACCC | 425 | GGGUGCAU CUGAUGAG GCCGUUAGGC CGAA IAUCCCUG | 1517 |
| 324 | AUCUAUGC A CCCAUCUG | 426 | CAGAUGGG CUGAUGAG GCCGUUAGGC CGAA ICAUAGAU | 1518 |
| 326 | CUAUGCAC C CAUCUGUG | 427 | CACAGAUG CUGAUGAG GCCGUUAGGC CGAA IUGCAUAG | 1519 |
| 327 | UAUGCACC C AUCUGUGU | 428 | ACACAGAU CUGAUGAG GCCGUUAGGC CGAA IGUGCAUA | 1520 |
| 328 | AUGCACCC A UCUGUGUG | 429 | CACACAGA CUGAUGAG GCCGUUAGGC CGAA IGGUGCAU | 1521 |
| 332 | CACCCAUC U GUGUGACU | 430 | AGUCACAC CUGAUGAG GCCGUUAGGC CGAA IAUGGGUG | 1522 |
| 339 | UGUGUGAC U GUGUGGUA | 431 | UACCACAC CUGAUGAG GCCGUUAGGC CGAA IUCACACA | 1523 |
| 349 | UGUGGUAC C CUCUCUGG | 432 | CCAGAGAG CUGAUGAG GCCGUUAGGC CGAA IUACCACA | 1524 |
| 350 | GUGGUACC C UCUCUGGG | 433 | CCCAGAGA CUGAUGAG GCCGUUAGGC CGAA IGUACCAC | 1525 |
| 352 | UGGUACCC U CUCUGGGG | 434 | CCCCAGAG CUGAUGAG GCCGUUAGGC CGAA IGGUACCA | 1526 |
| 353 | GUACCCUC U CUGGGGAG | 435 | CUCCCCAG CUGAUGAG GCCGUUAGGC CGAA IAGGGUAC | 1527 |
| 355 | ACCCUCUC U GGGGAGGC | 436 | GCCUCCCC CUGAUGAG GCCGUUAGGC CGAA IAGAGGGU | 1528 |
| 364 | GGGGAGGC A UUAUGUAU | 437 | AUACAUAA CUGAUGAG GCCGUUAGGC CGAA ICCUCCCC | 1529 |
| 381 | AUUAUUUC C GGAUCACU | 438 | AGUGAUCC CUGAUGAG GCCGUUAGGC CGAA IAAAUAAU | 1530 |
| 387 | UCCGGAUC A CUCCUGGC | 439 | CCCAGGAG CUGAUGAG GCCGUUAGGC CGAA IAUCCGGA | 1531 |
| 389 | CGGAUCAC U CCUGGCAG | 440 | CUGCCAGG CUGAUGAG GCCGUUAGGC CGAA IUGAUCCG | 1532 |
| 391 | GAUCACUC C UGGCAGCA | 441 | UGCUGCCA CUGAUGAG GCCGUUAGGC CGAA IAGUGAUC | 1533 |
| 392 | AUCACUCC U GGCAGCAA | 442 | UUGCUGCC CUGAUGAG GCCGUUAGGC CGAA IGAGUGAU | 1534 |
| 396 | CUCCUGGC A GCAACGGA | 443 | UCCGUUGC CUGAUGAG GCCGUUAGGC CGAA ICCAGGAG | 1535 |
| 399 | CUGGCAGC A ACGGAGAA | 444 | UUCUCCGU CUGAUGAG GCCGUUAGGC CGAA ICUGCCAG | 1536 |
| 412 | AGAAAAAC U CCAGGAAG | 445 | CUUCCUGG CUGAUGAG GCCGUUAGGC CGAA IUUUUUCU | 1537 |
| 414 | AAAAACUC C AGGAAGUG | 446 | CACUUCCU CUGAUGAG GCCGUUAGGC CGAA IAGUUUUU | 1538 |
| 415 | AAAACUCC A GGAAGUGU | 447 | ACACUUCC CUGAUGAG GCCGUUAGGC CGAA IGAGUUUU | 1539 |
| 430 | GUUUGGUC A AAGGAAAA | 448 | UUUUCCUU CUGAUGAG GCCGUUAGGC CGAA IACCAAAC | 1540 |
| 453 | AUGAAUUC A UUGAGCCU | 449 | AGGCUCAA CUGAUGAG GCCGUUAGGC CGAA IAAUUCAU | 1541 |
| 460 | CAUUGAGC C UCUUUGCU | 450 | AGCAAAGA CUGAUGAG GCCGUUAGGC CGAA ICUCAAUG | 1542 |
| 461 | AUUGAGCC U CUUUGCUG | 451 | CAGCAAAG CUGAUCAG GCCGUUAGGC CGAA IGCUCAAU | 1543 |
| 463 | UGAGCCUC U UUGCUGCC | 452 | GGCAGCAA CUGAUGAG GCCGUUAGGC CGAA IAGGCUCA | 1544 |
| 468 | CUCUUUGC U GCCAUUUC | 453 | GAAAUGGC CUGAUGAG GCCGUUAGGC CGAA ICAAAGAG | 1545 |
| 471 | UUUGCUGC C AUUUCUGG | 454 | CCAGAAAU CUGAUGAG GCCGUUAGGC CGAA ICAGCAAA | 1546 |
| 472 | UUGCUGCC A UUUCUGGA | 455 | UCCAGAAA CUGAUGAG GCCGUUAGGC CGAA IGCAGCAA | 1547 |
| 477 | GCCAUUUC U GGAAUGAU | 456 | AUCAUUCC CUGAUGAG GCCGUUAGGC CGAA IAAAUGGC | 1548 |
| 488 | AAUGAUUC U UUCAAUCA | 457 | UGAUUGAA CUGAUGAG GCCGUUAGGC CGAA IAAUCAUU | 1549 |
| 492 | AUUCUUUC A AUCAUGGA | 458 | UCCAUGAU CUGAUGAG GCCGUUAGGC CGAA IAAAGAAU | 1550 |
| 496 | UUUCAAUC A UGGACAUA | 459 | UAUGUCCA CUGAUGAG GCCGUUAGGC CGAA IAUUGAAA | 1551 |
| 502 | UCAUGGAC A UACUUAAU | 460 | AUUAAGUA CUGAUGAG GCCGUUAGGC CGAA IUCCAUGA | 1552 |
| 506 | GGACAUAC U UAAUAUUA | 461 | UAAUAUUA CUGAUGAG GCCGUUAGGC CGAA IUAUGUCC | 1553 |
| 522 | AAAAUUUC C CAUUUUUU | 462 | AAAAAAUG CUGAUGAG GCCGUUAGGC CGAA IAAAUUUU | 1554 |
| 523 | AAAUUUCC C AUUUUUUA | 463 | UAAAAAAU CUGAUGAG GCCGUUAGGC CGAA IGAAAUUU | 1555 |
| 524 | AAUUUCCC A UUUUUUAA | 464 | UUAAAAAA CUGAUGAG GCCGUUAGGC CGAA IGGAAAUU | 1556 |
| 545 | GGAGAGUC U GAAUUUUA | 465 | UAAAAUUC CUGAUGAG GCCGUUAGGC CGAA IACUCUCC | 1557 |
| 561 | AUUAGAGC U CACACACC | 466 | GGUGUGUG CUGAUGAG GCCGUUAGGC CGAA ICUCUAAU | 1558 |
| 563 | UAGAGCUC A CACACCAU | 467 | AUGGUGUG CUGAUGAG GCCGUUAGGC CGAA IAGCUCUA | 1559 |
| 565 | GAGCUCAC A CACCAUAU | 468 | AUAUGGUG CUGAUGAG GCCGUUAGGC CGAA IUGAGCUC | 1560 |
| 567 | GCUCACAC A CCAUAUAU | 469 | AUAUAUGG CUGAUGAG GCCGUUAGGC CGAA IUGUGAGC | 1561 |
| 569 | UCACACAC C AUAUAUUA | 470 | UAAUAUAU CUGAUGAG GCCGUUAGGC CGAA IUGUGUGA | 1562 |
| 570 | CACACACC A UAUAUUAA | 471 | UUAAUAUA CUGAUGAG GCCGUUAGGC CGAA IGUGUGUG | 1563 |
| 580 | AUAUUAAC A UAUACAAC | 472 | GUUGUAUA CUGAUGAG GCCGUUAGGC CGAA IUUAAUAU | 1564 |
| 586 | ACAUAUAC A ACUGUGAA | 473 | UUCACAGU CUGAUGAG GCCGUUAGGC CGAA IUAUAUGU | 1565 |
| 589 | UAUACAAC U GUGAACCA | 474 | UGGUUCAC CUGAUGAG GCCGUUAGGC CGAA IUUGUAUA | 1566 |
| 596 | CUGUGAAC C AGCUAAUC | 475 | GAUUAGCU CUGAUGAG GCCGUUAGGC CGAA IUUCACAG | 1567 |
| 597 | UGUGAACC A GCUAAUCC | 476 | GGAUUAGC CUGAUGAG GCCGUUAGGC CGAA IGUUCACA | 1568 |
| 600 | GAACCAGC U AAUCCCUC | 477 | GAGGGAUU CUGAUGAG GCCGUUAGGC CGAA ICUGGUUC | 1569 |
| 605 | AGCUAAUC C CUCUGAGA | 478 | UCUCAGAG CUGAUGAG GCCGUUAGGC CGAA IAUUAGCU | 1570 |
| 606 | GCUAAUCC C UCUGAGAA | 479 | UUCUCAGA CUGAUGAG GCCGUUAGGC CGAA IGAUUAGC | 1571 |
| 607 | CUAAUCCC U CUGAGAAA | 480 | UUUCUCAG CUGAUGAG GCCGUUAGGC CGAA IGGAUUAG | 1572 |
| 609 | AAUCCCUC U GAGAAAAA | 481 | UUUUUCUC CUGAUGAG GCCGUUAGGC CGAA IAGGGAUU | 1573 |
| 619 | AGAAAAAC U CCCCAUCU | 482 | AGAUGGGG CUGAUGAG GCCGUUAGGC CGAA IUUUUUCU | 1574 |
| 621 | AAAAACUC C CCAUCUAC | 483 | GUAGAUGG CUGAUGAG GCCGUUAGGC CGAA IAGUUUUU | 1575 |
| 622 | AAAACUCC C CAUCUACC | 484 | GGUAGAUG CUGAUGAG GCCGUUAGGC CGAA IGAGUUUU | 1576 |
| 623 | AAACUCCC C AUCUACCC | 485 | GGGUAGAU CUGAUGAG GCCGUUAGGC CGAA IGGAGUUU | 1577 |
| 624 | AACUCCCC A UCUACCCA | 486 | UGGGUAGA CUGAUGAG GCCGUUAGGC CGAA IGGGAGUU | 1578 |
| 627 | UCCCCAUC U ACCCAAUA | 487 | UAUUGGGU CUGAUGAG GCCGUUAGGC CGAA IAUGGGGA | 1579 |
| 630 | CCAUCUAC C CAAUACUG | 488 | CAGUAUUG CUGAUGAG GCCGUUAGGC CGAA IUAGAUGG | 1580 |
| 631 | CAUCUACC C AAUACUGU | 489 | ACAGUAUU CUGAUGAG GCCGUUAGGC CGAA IGUAGAUG | 1581 |
| 632 | AUCUACCC A AUACUGUU | 490 | AACAGUAU CUGAUGAG GCCGUUAGGC CGAA IGGUAGAU | 1582 |
| 637 | CCCAAUAC U GUUACAGC | 491 | GCUGUAAC CUGAUGAG GCCGUUAGGC CGAA IUAUUGGG | 1583 |
| 643 | ACUGUUAC A GCAUACAA | 492 | CUGUAUGC CUGAUGAG GCCGUUAGGC CGAA IUAACAGU | 1584 |
| 646 | GUUACAGC A UACAAUCU | 493 | AGAUUGUA CUGAUGAG GCCGUUAGGC CGAA ICUGUAAC | 1585 |
| 650 | CAGCAUAC A AUCUCUGU | 494 | ACAGAGAU CUGAUGAG GCCGUUAGGC CGAA IUAUGCUG | 1586 |
| 654 | AUACAAUC U CUGUUCUU | 495 | AAGAACAG CUGAUGAG GCCGUUAGGC CGAA IAUUGUAU | 1587 |
| 656 | ACAAUCUC U GUUCUUGG | 496 | CCAAGAAC CUGAUGAG GCCGUUAGGC CGAA IAGAUUGU | 1588 |
| 661 | CUCUGUUC U UGGGCAUU | 497 | AAUGCCCA CUGAUGAG GCCGUUAGGC CGAA IAACAGAG | 1589 |
| 667 | UCUUGGGC A UUUUGUCA | 498 | UGACAAAA CUGAUGAG GCCGUUAGGC CGAA ICCCAAGA | 1590 |
| 675 | AUUUUGUC A GUGAUGCU | 499 | AGCAUCAC CUGAUGAG GCCGUUAGGC CGAA IACAAAAU | 1591 |
| 683 | AGUGAUGC U GAUCUUUG | 500 | CAAAGAUC CUGAUGAG GCCGUUAGGC CGAA ICAUCACU | 1592 |
| 688 | UGCUGAUC U UUGCCUUC | 501 | GAAGGCAA CUGAUGAG GCCGUUAGGC CGAA IAUCAGCA | 1593 |
| 693 | AUCUUUGC C UUCUUCCA | 502 | UGGAAGAA CUGAUGAG GCCGUUAGGC CGAA ICAAAGAU | 1594 |
| 694 | UCUUUGCC U UCUUCCAG | 503 | CUGGAAGA CUGAUGAG GCCGUUAGGC CGAA IGCAAAGA | 1595 |
| 697 | UUGCCUUC U UCCAGGAA | 504 | UUCCUGGA CUGAUGAG GCCGUUAGGC CGAA IAAGGCAA | 1596 |
| 700 | CCUUCUUC C AGGAACUU | 505 | AAGUUCCU CUGAUGAG GCCGUUAGGC CGAA IAAGAAGG | 1597 |
| 701 | CUUCUUCC A GGAACUUG | 506 | CAAGUUCC CUGAUGAG GCCGUUAGGC CGAA IGAAGAAG | 1598 |
| 707 | CCAGGAAC U UGUAAUAG | 507 | CUAUUACA CUGAUGAG GCCGUUAGGC CGAA IUUCCUGG | 1599 |
| 717 | GUAAUAGC U GGCAUCGU | 508 | ACGAUGCC CUGAUGAG GCCGUUAGGC CGAA ICUAUUAC | 1600 |
| 721 | UAGCUGGC A UCGUUGAG | 509 | CUCAACGA CUGAUGAG GCCGUUAGGC CGAA TCCAGCUA | 1601 |
| 751 | GAACGUGC U CCAGACCC | 510 | GGGUCUGG CUGAUGAG GCCGUUAGGC CGAA ICACGUUC | 1602 |
| 753 | ACGUGCUC C AGACCCAA | 511 | UUGGGUCU CUGAUGAG GCCGUUAGGC CGAA IAGCACGU | 1603 |
| 754 | CGUGCUCC A GACCCAAA | 512 | UUUGGGUC CUGAUGAG GCCGUUAGGC CGAA IGAGCACG | 1604 |
| 758 | CUCCAGAC C CAAAUCUA | 513 | UAGAUUUG CUGAUGAG GCCGUUAGGC CGAA IUCUGGAG | 1605 |
| 759 | UCCAGACC C AAAUCUAA | 514 | UUAGAUUU CUGAUGAG GCCGUUAGGC CGAA IGUCUGGA | 1606 |
| 760 | CCAGACCC A AAUCUAAC | 515 | GUUAGAUU CUGAUGAG GCCGUUAGGC CGAA IGGUCUGG | 1607 |
| 765 | CCCAAAUC U AACAUAGU | 516 | ACUAUGUU CUGAUGAG GCCGUUAGGC CGAA IAUUUGGG | 1608 |
| 769 | AAUCUAAC A UAGUUCUC | 517 | GAGAACUA CUGAUGAG GCCGUUAGGC CGAA IUUAGAUU | 1609 |
| 776 | CAUAGUUC U CCUGUCAG | 518 | CUGACAGG CUGAUGAG GCCGUUAGGC CGAA IAACUAUG | 1610 |
| 778 | UAGUUCUC C UGUCAGCA | 519 | UGCUGACA CUGAUGAG GCCGUUAGGC CGAA IAGAACUA | 1611 |
| 779 | AGUUCUCC U GUCAGCAG | 520 | CUGCUGAC CUGAUGAG GCCGUUAGGC CGAA IGAGAACU | 1612 |
| 783 | CUCCUGUC A GCAGAAGA | 521 | UCUUCUGC CUGAUGAG GCCGUUAGGC CGAA IACAGGAG | 1613 |
| 786 | CUGUCAGC A GAAGAAAA | 522 | UUUUCUUC CUGAUGAG GCCGUUAGGC CGAA ICUGACAG | 1614 |
| 803 | AAAAGAAC A GACUAUUG | 523 | CAAUAGUC CUGAUGAG GCCGUUAGGC CGAA IUUCUUUU | 1615 |
| 807 | GAACAGAC U AUUGAAAU | 524 | AUUUCAAU CUGAUGAG GCCGUUAGGC CGAA IUCUGUUC | 1616 |
| 836 | GGUUGGGC U AACUGAAA | 525 | UUUCAGUU CUGAUGAG GCCGUUAGGC CGAA ICCCAACC | 1617 |
| 840 | GGGCUAAC U GAAACAUC | 526 | GAUGUUUC CUGAUGAG GCCGUUAGGC CGAA IUUAGCCC | 1618 |
| 846 | ACUGAAAC A UCUUCCCA | 527 | UGGGAAGA CUGAUGAG GCCGUUAGGC CGAA IUUUCAGU | 1619 |
| 849 | GAAACAUC U UCCCAACC | 528 | GGUUGGGA CUGAUGAG GCCGUUAGGC CGAA IAUGUUUC | 1620 |
| 852 | ACAUCUUC C CAACCAAA | 529 | UUUGGUUG CUGAUGAG GCCGUUAGGC CGAA IAAGAUGU | 1621 |
| 853 | CAUCUUCC C AACCAAAG | 530 | CUUUGGUU CUGAUGAG GCCGUUAGGC CGAA IGAAGAUG | 1622 |
| 854 | AUCUUCCC A ACCAAAGA | 531 | UCUUUGGU CUGAUGAG GCCGUUAGGC CGAA IGGAAGAU | 1623 |
| 857 | UUCCCAAC C AAAGAAUG | 532 | CAUCCUUG CUGAUGAG GCCGUUAGGC CGAA IUUGGGAA | 1624 |
| 858 | UCCCAACC A AAGAAUGA | 533 | UCAUUCUU CUGAUGAG GCCGUUAGGC CGAA IGUUGGGA | 1625 |
| 874 | AAGAAGAC A UUGAAAUU | 534 | AAUUUCAA CUGAUGAG GCCGUUAGGC CGAA IUCUUCUU | 1626 |
| 887 | AAUUAUUC C AAUCCAAG | 535 | CUUGGAUU CUGAUGAG GCCGUUAGGC CGAA IAAUAAUU | 1627 |
| 888 | AUUAUUCC A AUCCAAGA | 536 | UCUUGGAU CUGAUGAG GCCGUUAGGC CGAA IGAAUAAU | 1628 |
| 892 | UUCCAAUC C AAGAAGAG | 537 | CUCUUCUU CUGAUGAG GCCGUUAGGC CGAA IAUUGGAA | 1629 |
| 893 | UCCAAUCC A AGAAGAGG | 538 | CCUCUUCU CUGAUGAG GCCGUUAGGC CGAA IGAUUGGA | 1630 |
| 915 | GAAGAAAC A GAGACGAA | 539 | UUCGUCUC CUGAUGAG GCCGUUAGGC CGAA IUUUCUUC | 1631 |
| 925 | AGACGAAC U UUCCAGAA | 540 | UUCUGGAA CUGAUGAG GCCGUUAGGC CGAA IUUCGUCU | 1632 |
| 929 | GAACUUUC C AGAACCUC | 541 | GAGGUUCU CUGAUGAG GCCGUUAGGC CGAA IAAAGUUC | 1633 |
| 930 | AACUUUCC A GAACCUCC | 542 | GGAGGUUC CUGAUGAG GCCGUUAGGC CGAA IGAAAGUU | 1634 |
| 935 | UCCAGAAC C UCCCCAAG | 543 | CUUGGGGA CUGAUGAG GCCGUUAGGC CGAA IUUCUGGA | 1635 |
| 936 | CCAGAACC U CCCCAAGA | 544 | UCUUGGGG CUGAUGAG GCCGUUAGGC CGAA IGUUCUGG | 1636 |
| 938 | AGAACCUC C CCAAGAUC | 545 | GAUCUUGG CUGAUGAG GCCGUUAGGC CGAA IAGGUUCU | 1637 |
| 939 | GAACCUCC C CAAGAUCA | 546 | UGAUCUUG CUGAUGAG GCCGUUAGGC CGAA IGAGGUUC | 1638 |
| 940 | AACCUCCC C AAGAUCAG | 547 | CUGAUCUU CUGAUGAG GCCGUUAGGC CGAA IGGAGGUU | 1639 |
| 941 | ACCUCCCC A AGAUCAGC | 548 | CCUGAUCU CUGAUGAG GCCGUUAGGC CGAA IGGGAGGU | 1640 |
| 947 | CCAAGAUC A GGAAUCCU | 549 | AGGAUUCC CUGAUGAG GCCGUUAGGC CGAA IAUCUUGG | 1641 |
| 954 | CAGGAAUC C UCACCAAU | 550 | AUUGGUGA CUGAUCAG GCCGUUAGGC CGAA IAUUCCUG | 1642 |
| 955 | AGGAAUCC U CACCAAUA | 551 | UAUUCGUG CUGAUGAG GCCGUUAGGC CGAA IGAUUCCU | 1643 |
| 957 | GAAUCCUC A CCAAUAGA | 552 | UCUAUUGG CUGAUGAG GCCGUUAGGC CGAA IAGGAUUC | 1644 |
| 959 | AUCCUCAC C AAUAGAAA | 553 | UUUCUAUU CUGAUGAG GCCGUUAGGC CGAA IUGAGGAU | 1645 |
| 960 | UCCUCACC A AUAGAAAA | 554 | UUUUCUAU CUGAUGAG GCCGUUAGGC CGAA IGUGAGGA | 1646 |
| 973 | AAAAUGAC A GCUCUCCU | 555 | AGGAGAGC CUGAUGAG GCCGUUAGGC CGAA IUCAUUUU | 1647 |
| 976 | AUGACAUC U CUCCUUAA | 556 | UUAAGGAG CUGAUGAG GCCGUUAGGC CGAA ICUGUCAU | 1648 |
| 978 | GACACCUC U CCUUAAGU | 557 | ACUUAAGG CUGAUGAG GCCGUUAGGC CGAA IAGCUGUC | 1649 |
| 980 | CAGCUCUC C UUAAGUGA | 558 | UCACUUAA CUGAUGAG GCCGUUAGGC CGAA IAGAGCUG | 1650 |
| 981 | AGCUCUCC U UAAGUGAU | 559 | AUCACUUA CUGAUGAG GCCGUUAGGC CGAA IGAGAGCU | 1651 |
| 993 | GUGAUUUC U UCUGUUUU | 560 | AAAACAGA CUGAUGAG GCCGUUAGGC CGAA IAAAUCAC | 1652 |
| 996 | AUUUCUUC U GUUUUCUG | 561 | CAGAAAAC CUGAUGAG GCCGUUAGGC CGAA IAAGAAAU | 1653 |
| 1003 | CUGUUUUC U GUUUCCUU | 562 | AAGGAAAC CUGAUGAG GCCGUUAGGC CGAA IAAAACAG | 1654 |
| 1009 | UCUGUUUC C UUUUUUAA | 563 | UUAAAAAA CUGAUGAG GCCGUUAGGC CGAA IAAACAGA | 1655 |
| 1010 | CUGUUUCC U UUUUUAAA | 564 | UUUAAAAA CUGAUGAG GCCGUUAGGC CGAA IGAAACAG | 1656 |
| 1020 | UUUUAAAC A UUAGUGUU | 565 | AACACUAA CUGAUGAG GCCGUUAGGC CGAA IUUUAAAA | 1657 |
| 1030 | UAGUGUUC A UAGCUUCC | 566 | GGAAGCUA CUGAUGAG GCCGUUAGGC CGAA IAACACUA | 1658 |
| 1035 | UUCAUAGC U UCCAAGAG | 567 | CUCUUGGA CUGAUGAG GCCGUUAGGC CGAA ICUAUGAA | 1659 |
| 1038 | AUAGCUUC C AAGAGACA | 568 | UGUCUCUU CUGAUGAG GCCGUUAGGC CGAA IAAGCUAU | 1660 |
| 1039 | UAGCUUCC A AGAGACAU | 569 | AUGUCUCU CUGAUGAG GCCGUUAGGC CGAA IGAAGCUA | 1661 |
| 1046 | CAAGAGAC A UGCUGACU | 570 | AGUCAGCA CUGAUGAG GCCGUUAGGC CGAA IUCUCUUG | 1662 |
| 1050 | AGACAUGC U GACUUUCA | 571 | UGAAAGUC CUGAUGAG GCCGUUAGGC CGAA ICAUGUCU | 1663 |
| 1054 | AUGCUGAC U UUCAUUUC | 572 | GAAAUGAA CUGAUGAG GCCGUUAGGC CGAA IUCAGCAU | 1664 |
| 1058 | UGACUUUC A UUUCUUGA | 573 | UCAAGAAA CUGAUGAG GCCGUUAGGC CGAA IAAAGUCA | 1665 |
| 1063 | UUCAUUUC U UGAGGUAC | 574 | GUACCUCA CUGAUGAG GCCGUUAGGC CGAA IAAAUGAA | 1666 |
| 1072 | UGAGGUAC U CUGCACAU | 575 | AUGUGCAG CUGAUGAG GCCGUUAGGC CGAA IUACCUCA | 1667 |
| 1074 | AGGUACUC U GCACAUAC | 576 | GUAUGUGC CUGAUGAG GCCGUUAGGC CGAA IAGUACCU | 1668 |
| 1077 | UACUCUGC A CAUACGCA | 577 | UGCGUAUG CUGAUGAG GCCGUUAGGC CGAA ICAGAGUA | 1669 |
| 1079 | CUCUGCAC A UACGCACC | 578 | GGUGCGUA CUGAUGAG GCCGUUAGGC CGAA IUGCAGAG | 1670 |
| 1085 | ACAUACCC A CCACAUCU | 579 | AGAUGUGG CUGAUGAG GCCGUUAGGC CGAA ICGUAUGU | 1671 |
| 1087 | AUACGCAC C ACAUCUCU | 580 | AGAGAUGU CUGAUGAG GCCGUUAGGC CGAA IUGCGUAU | 1672 |
| 1088 | UACGCACC A CAUCUCUA | 581 | UAGAGAUG CUGAUGAG GCCGUUAGGC CGAA IGUGCGUA | 1673 |
| 1090 | CGCACCAC A UCUCUAUC | 582 | GAUAGAGA CUGAUGAG GCCGUUAGGC CGAA IUGGUGCG | 1674 |
| 1093 | ACCACAUC U CUAUCUGG | 583 | CCAGAUAG CUGAUGAG GCCGUUAGGC CGAA IAUGUGGU | 1675 |
| 1095 | CACAUCUC U AUCUGGCC | 584 | GGCCAGAU CUGAUGAG GCCGUUAGGC CGAA IAGAUGUG | 1676 |
| 1099 | UCUCUAUC U GGCCUUUG | 585 | CAAAGGCC CUGAUGAG GCCGUUAGGC CGAA IAUAGAGA | 1677 |
| 1103 | UAUCUGGC C UUUGCAUG | 586 | CAUGCAAA CUGAUGAG GCCGUUAGGC CGAA ICCAGAUA | 1678 |
| 1104 | AUCUGGCC U UUGCAUGG | 587 | CCAUGCAA CUGAUGAG GCCGUUAGGC CGAA IGCCAGAU | 1679 |
| 1109 | GCCUUUGC A UGGAGUGA | 588 | UCACUCCA CUGAUGAG GCCGUUAGGC CGAA ICAAAGGC | 1680 |
| 1119 | GGAGUGAC C AUAGCUCC | 589 | GGAGCUAU CUGAUGAG GCCGUUAGGC CGAA IUCACUCC | 1681 |
| 1120 | GAGUGACC A UAGCUCCU | 590 | AGGAGCUA CUGAUGAG GCCGUUAGGC CGAA IGUCACUC | 1682 |
| 1125 | ACCAUAGC U CCUUCUCU | 591 | AGAGAAGG CUGAUGAG GCCGUUAGGC CGAA ICUAUGGU | 1683 |
| 1127 | CAUAGCUC C UUCUCUCU | 592 | AGAGAGAA CUGAUGAG GCCGUUAGGC CGAA IAGCUAUG | 1684 |
| 1128 | AUAGCUCC U UCUCUCUU | 593 | AAGAGAGA CUGAUGAG GCCGUUAGGC CGAA IGAGCUAU | 1685 |
| 1131 | GCUCCUUC U CUCUUACA | 594 | UGUAAGAG CUGAUGAG GCCGUUAGGC CGAA IAAGGAGC | 1686 |
| 1133 | UCCUUCUC U CUUACAUU | 595 | AAUGUAAG CUGAUGAG GCCGUUAGGC CGAA IAGAAGGA | 1687 |
| 1135 | CUUCUCUC U UACAUUGA | 596 | UCAAUGUA CUGAUGAG GCCGUUAGGC CGAA IAGAGAAG | 1688 |
| 1139 | UCUCUUAC A UUGAAUGU | 597 | ACAUUCAA CUGAUGAG GCCGUUAGGC CGAA IUAAGAGA | 1689 |
| 1160 | AAUGUAGC C AUUGUAGC | 598 | GCUACAAU CUGAUGAG GCCGUUAGGC CGAA ICUACAUU | 1690 |
| 1161 | AUGUAGCC A UUGUAGCA | 599 | UGCUACAA CUGAUGAG GCCGUUAGGC CGAA IGCUACAU | 1691 |
| 1169 | AUUGUAGC A GCUUGUGU | 600 | ACACAAGC CUGAUGAG GCCGUUAGGC CGAA ICUACAAU | 1692 |
| 1172 | GUAGCAGC U UGUGUUGU | 601 | ACAACACA CUGAUGAG GCCGUUAGGC CGAA ICUGCUAC | 1693 |
| 1182 | GUGUUGUC A CGCUUCUU | 602 | AAGAAGCG CUGAUGAG GCCGUUAGGC CGAA IACAACAC | 1694 |
| 1186 | UGUCACGC U UCUUCUUU | 603 | AAGAAGAG CUGAUGAG GCCGUUAGGC CGAA ICGUGACA | 1695 |
| 1189 | CACGCUUC U CUTIUUGA | 604 | UCAAAAGA CUGAUGAG GCCGUUAGGC CGAA IAAGCGUG | 1696 |
| 1192 | GCUUCUUC U UUUGAGCA | 605 | UGCUCAAA CUGAUGAG GCCGUUAGGC CGAA IAAGAAGC | 1697 |
| 1200 | UUUGAGCA A CUUUUCUU | 606 | AAGAAAGU CUGAUGAG GCCGUUAGGC CGAA ICUCAAAA | 1698 |
| 1203 | UGAGCAAC U UUCUUACA | 607 | UGUAAGAA CUGAUGAG GCCGUUAGGC CGAA IUUGCUCA | 1699 |
| 1207 | CAACUUUC U UACACUGA | 608 | UCAGUGUA CUGAUGAG GCCGUUAGGC CGAA IAAAGUUG | 1700 |
| 1211 | UUUCUUAC A CUGAAGAA | 609 | UUCUUCAG CUGAUGAG GCCGUUAGGC CGAA IUAAGAAA | 1701 |
| 2213 | UCUUACAC U GAAGAAAG | 610 | CUUUCUUC CUGAUGAG GCCGUUAGGC CGAA IUGUAAGA | 1702 |
| 1224 | AGAAAGGC A GAAUGAGU | 611 | ACUCAUUC CUGAUGAG GCCGUUAGGC CGAA ICCUUUCU | 1703 |
| 1235 | AUGAGUGC U UCAGAAUG | 612 | CAUUCUGA CUGAUGAG GCCGUUAGGC CGAA ICACUCAU | 1704 |
| 1238 | AGUGCUUC A GAAUGUGA | 613 | UCACAUUC CUGAUGAG GCCGUUAGGC CGAA IAAGCACU | 1705 |
| 1251 | GUGAUUUC C UACUAACC | 614 | GGUUAGUA CUGAUGAG GCCGUUAGGC CGAA IAAAUCAC | 1706 |
| 1252 | UGAUUUCC U ACUAACCU | 615 | AGGUUAGU CUGAUGAG GCCGUUAGGC CGAA IGAAAUCA | 1707 |
| 1255 | UUUCCUAC U AACCUGUU | 616 | AACAGGUU CUGAUGAG GCCGUUAGGC CGAA IUAGGAAA | 1708 |
| 1259 | CUACUAAC C UGUUCCUU | 617 | AAGGAACA CUGAUGAG GCCGUUAGGC CGAA IUUAGUAG | 1709 |
| 1260 | UACUAACC U GUUCCUUG | 618 | CAAGGAAC CUGAUGAG GCCGUUAGGC CGAA IGUUAGUA | 1710 |
| 1265 | ACCUGUUC C UUGGAUAG | 619 | CUAUCCAA CUGAUGAG GCCGUUAGGC CGAA IAACAGGU | 1711 |
| 1266 | CCUGUUCC U UGGAUAGG | 620 | CCUAUCCA CUGAUGAG GCCGUUAGGC CGAA IGAACAGG | 1712 |
| 1276 | GGAUAGGC U ThIUAGUA | 621 | UACUAAAA CUGAUGAG GCCGUUAGGC CGAA ICCUAUCC | 1713 |
| 1302 | UUUUUGUC A UUUUCUCC | 622 | GGAGAAAA CUGAUGAG GCCGUUAGGC CGAA IACAAAAA | 1714 |
| 1308 | UCAUUUUC U CCAUCAGC | 623 | GCUGAUGG CUGAUGAG GCCGUUAGGC CGAA IAAAAUGA | 1715 |
| 1310 | AUUUUCUC C AUCAGCAA | 624 | UUGCUGAU CUGAUGAG GCCGUUAGGC CGAA IAGAAAAU | 1716 |
| 1311 | UUUCUCCA U UCAGCAAC | 625 | GUUGCUGA CUGAUGAG GCCGUUAGGC CGAA IGAGAAAA | 1717 |
| 1314 | UCUCCAUC A GCAACCAG | 626 | CUGGUUGC CUGAUGAG GCCGUUAGGC CGAA IAUGGAGA | 1718 |
| 1317 | CCAUCAGC A ACCAGGGA | 627 | UCCCUGGU CUGAUGAG GCCGUUAGGC CGAA ICUGAUGG | 1719 |
| 1320 | UCAGCAAC C AGGGAGAC | 628 | GUCUCCCU CUGAUGAG GCCGUUAGGC CGAA IUUGCUGA | 1720 |
| 1321 | CAGCAACC A GGGAGACU | 629 | AGUCUCCC CUGAUGAG GCCGUUAGGC CGAA IGUUGCUG | 1721 |
| 1329 | AGGGAGAC U GCACCUGA | 630 | UCAGGUGC CUGAUGAG GCCGUUAGGC CGAA IUCUCCCU | 1722 |
| 1332 | GAGACUGC A CCUGAUGG | 631 | CCAUCAGG CUGAUGAG GCCGUUAGGC CGAA ICAGUCUC | 1723 |
| 1334 | GACUGCAC C UGAUGGAA | 632 | UUCCAUCA CUGAUGAG GCCGUUAGGC CGAA IUGCAGUC | 1724 |
| 1335 | ACUGCACC U GAUGGAAA | 633 | UUUCCAUC CUGAUGAG GCCGUUAGGC CGAA IGUGCAGU | 1725 |
| 1355 | UAUAUGAC U GCUUCAUG | 634 | CAUGAAGC CUGAUGAG GCCGUUAGGC CGAA IUCAUAUA | 1726 |
| 1358 | AUGACUGC U UCAUGACA | 635 | UGUCAUGA CUGAUGAG GCCGUUAGGC CGAA ICAGUCAU | 1727 |
| 1361 | ACUGCUUC A UGACAUUC | 636 | GAAUGUCA CUGAUGAG GCCGUUAGGC CGAA IAAGCAGU | 1728 |
| 1366 | UUCAUGAC A UUCCUAAA | 637 | UUUAGGAA CUGAUGAG GCCGUUAGGC CGAA IUCAUGAA | 1729 |
| 1370 | UGACAUUC C UAAACUAU | 638 | AUAGUUUA CUGAUGAG GCCGUUAGGC CGAA IAAUGUCA | 1730 |
| 1371 | GACAUUCC U AAACUAUC | 639 | GAUAGUUU CUGAUGAG GCCGUUAGGC CGAA IGAAUGUC | 1731 |
| 1376 | UCCUAAAC U AUCUUUUU | 640 | AAAAAGAU CUGAUGAG GCCGUUAGGC CGAA IUUUAGGA | 1732 |
| 1380 | AAACUAUC U UUUUUUUA | 641 | UAAAAAAA CUGAUGAG GCCGUUAGGC CGAA IAUAGUUU | 1733 |
| 1392 | UUUUAUUC C ACAUCUAC | 642 | GUAGAUGU CUGAUGAG GCCGUUAGGC CGAA IAAUAAAA | 1734 |
| 1393 | UUAUUCCA C CAUCUACG | 643 | CGUAGAUG CUGAUGAG GCCGUUAGGC CGAA IGAAUAAA | 1735 |
| 1395 | UAUUCCAC A UCUACGUU | 644 | AACGUAGA CUGAUGAG GCCGUUAGGC CGAA IUGGAAUA | 1736 |
| 1398 | UCCACAUC U ACGUUUUU | 645 | AAAAACGU CUGAUGAG GCCGUUAGGC CGAA IAUGUGGA | 1737 |
| 1416 | GUGGAGUC C CUUUUGCA | 646 | UGCAAAAG CUGAUGAG GCCGUUAGGC CGAA IACUCCAC | 1738 |
| 1417 | UGGAGUCC C UUUUGCAU | 647 | AUGCAAAA CUGAUGAG GCCGUUAGGC CGAA IGACUCCA | 1739 |
| 1418 | GGAGUCCC U UUUGCAUC | 648 | GAUGCAAA CUGAUGAG GCCGUUAGGC CGAA IGGACUCC | 1740 |
| 1424 | CCUUUGCA U UCAUUGUU | 649 | AACAAUGA CUGAUGAG GCCGUUAGGC CGAA ICAAAAGG | 1741 |
| 1427 | UUUGCAUC A UUGUUUUA | 650 | UAAAACAA CUGAUGAG GCCGUUAGGC CGAA IAUGCAAA | 1742 |
| 1458 | AAAAUAAC A ACUAGGGA | 651 | UCCCUAGU CUGAUGAG GCCGUUAGGC CGAA IUUAUUUU | 1743 |
| 1461 | AUAACAAC U AGGGACAA | 652 | UUGUCCCU CUGAUGAG GCCGUUAGGC CGAA IUUGUUAU | 1744 |
| 1468 | CUAGGUAC A AUACAGAA | 653 | UUCUGUAU CUGAUGAG GCCGUUAGGC CGAA IUCCCUAG | 1745 |
| 1473 | GACAAUAC A GAACCCAU | 654 | AUGGGUUC CUGAUGAG GCCGUUAGGC CGAA IUAGUGUC | 1746 |
| 1478 | UACAGAAC C CAUUCCAU | 659 | AUGGAAUG CUGAUGAG GCCGUUAGGC CGAA IUUCUGUA | 1747 |
| 1479 | ACAGAACC C AUUCCAUU | 656 | AAUGGAAU CUGAUGAG GCCGUUAGGC CGAA IGUUCUGU | 1748 |
| 1480 | CAGAACCC A UUCCAUUU | 657 | AAAUGGAA CUGAUGAG GCCGUUAGGC CGAA IGGUUCUG | 1749 |
| 1484 | ACCCAUUC C AUUUAUCU | 658 | AGAUAAAU CUGAUGAG GCCGUUAGGC CGAA IAAUGGGU | 1750 |
| 1485 | CCCAUUCC A UUUAUCUU | 659 | AAGAUAAA CUGAUGAG GCCGUUAGGC CGAA IGAAUGGG | 1751 |
| 1492 | CAUUUAUC U UUCUACAG | 660 | CUGUAGAA CUGAUGAG GCCGUUAGGC CGAA IAUAAAUG | 1752 |
| 1496 | UAUCUUUC U ACAGGGCU | 661 | AGCCCUGU CUGAUGAG GCCGUUAGGC CGAA IAAAGAUA | 1753 |
| 1499 | CUUUCUAC A GGGCUGAC | 662 | GUCAGCCC CUGAUGAG GCCGUUAGGC CGAA IUAGAAAG | 1754 |
| 1504 | UACAGGGC U GACAUUGU | 663 | ACAAUGUC CUGAUGAG GCCGUUAGGC CGAA ICCCUGUA | 1755 |
| 1508 | GGGCUGAC A UUGUGGCA | 664 | UGCCACAA CUGAUGAG GCCGUUAGGC CGAA IUCAGCCC | 1756 |
| 1516 | AUUGUGGC A CAUUCUUA | 665 | UAAGAAUG CUGAUGAG GCCGUUAGGC CGAA ICCACAAU | 1757 |
| 1518 | UGUGGCAC A UUCUUAGA | 666 | UCUAAGAA CUGAUGAG GCCGUUAGGC CGAA IUGCCACA | 1758 |
| 1522 | GCACAUUC U UAGAGUUA | 667 | UAACUCUA CUGAUGAG GCCGUUAGGC CGAA IAAUGUGC | 1759 |
| 1532 | AGAGUUAC C ACACCCCA | 668 | UGGGGUGU CUGAUGAG GCCGUUAGGC CGAA IUAACUCU | 1760 |
| 1533 | GAGUUACC A CACCCCAU | 669 | AUGGGGUG CUGAUGAG GCCGUUAGGC CGAA IGUAACUC | 1761 |
| 1535 | GUUACCAC A CCCCAUGA | 670 | UCAUGGGG CUGAUGAG GCCGUUAGGC CGAA IUGGUAAC | 1762 |
| 1537 | UACCACAC C CCAUGAGG | 671 | CCUCAUGG CUGAUGAG GCCGUUAGGC CGAA IUGUGGUA | 1763 |
| 1538 | ACCACACC C CAUGAGGG | 672 | CCCUCAUG CUGAUGAG GCCGUUAGGC CGAA IGUGUGGU | 1764 |
| 1539 | CCACACCC C AUGAGGGA | 673 | UCCCUCAU CUGAUGAG GCCGUUAGGC CGAA IGGUGUGG | 1765 |
| 1540 | CACACCCC A UGAGGGAA | 674 | UUCCCUCA CUGAUGAG GCCGUUAGGC CGAA IGGGUGUG | 1766 |
| 1551 | AGGGAAGC U CUAAAUAG | 675 | CUAUUUAG CUGAUGAG GCCGUUAGGC CGAA ICUUCCCU | 1767 |
| 1553 | GGAAGCUC U AAAUAGCC | 676 | GGCUAUUU CUGAUGAG GCCGUUAGGC CGAA IAGCUUCC | 1768 |
| 1561 | UAAAUAGC C AACACCCA | 677 | UGGGUGUU CUGAUGAG GCCGUUAGGC CGAA ICUAUUUA | 1769 |
| 1562 | AAAUAGCC A ACACCCAU | 678 | AUGGGUGU CUGAUGAG GCCGUUAGGC CGAA IGCUAUUU | 1770 |
| 1565 | UAGCCAAC A CCCAUCUG | 679 | CAGAUGGG CUGAUGAG GCCGUUAGGC CGAA IUUGGCUA | 1771 |
| 1567 | GCCAACAC C CAUCUGUU | 680 | AACAGAUG CUGAUGAG GCCGUUAGGC CGAA IUGUUGGC | 1772 |
| 1568 | CCAACACC C AUCUGUUU | 681 | AAACAGAU CUGAUGAG GCCGUUAGGC CGAA IGUGUUGG | 1773 |
| 1569 | CAACACCC A UCUGUUUU | 682 | AAAACAGA CUGAUGAG GCCGUUAGGC CGAA IGGUGUUG | 1774 |
| 1572 | CACCCAUC U GUUUUUUG | 683 | CAAAAAAC CUGAUGAG GCCGUUAGGC CGAA IAUGGGUG | 1775 |
| 1588 | GUAAAAAC A GCAUAGCU | 684 | AGCUAUGC CUGAUGAG GCCGUUAGGC CGAA UIUUUUAC | 1776 |

Input Sequence = HSCD20A. Cut Site = CH/.
Stem Length = 8. Core Sequence = CUGAUGAG X CGAA (X = GCCGUUAGGC or other stem II)
HSCD20A (Human mRNA for CD20 receptor (57); 1597 bp)

Underlined region can be any X sequence or linker, as previously described herein. I=Inosine

TABLE V
Human CD20 G-cleaver Ribozyme and Substrate Sequence

| | | | | Rz Seq |
|---|---|---|---|---|
| Pos | Substrate | Seq ID | Ribozyme | ID |
| 9 | AACAAACU C CACCCACU | 685 | AGUCGGUC UGAUG GCAUGCACUAUGC GCG AGUUUGUU | 1777 |
| 18 | CACCCACU G AACUCCGC | 686 | GCGCACUU UGAUC GCAUGCACUAUGC GCG ACUGCGUC | 1778 |
| 25 | UGAACUCC C CACCUACC | 687 | CCUACCUG UGAUC GCAUGCACUAUGC GCG GGAGUUCA | 1779 |
| 50 | CACCCCUU C AGAUUUCA | 688 | UCAAAUCU UCAUG GCAUGCACUAUGC GCG AAGGGCUG | 1780 |
| 57 | UGACAUUU C ACGCCUUG | 689 | CAAGGCCU UGAUG GCAUGCACUAUGC GCG AAAUCUCA | 1781 |
| 82 | GGAGUUUU G AGAGCAAA | 680 | UUUGCUCU UGAUG GCAUGCACUAUGC GCG AAAACUCC | 1782 |
| 93 | AGCAAAAU G ACAACACC | 691 | GGUGUUCU UGAUG GCAUGCACUAUGC GCG AUUUUCCU | 1783 |
| 141 | GAGCCAAU C AAACGCCC | 692 | CGCCCUUU UGAUG GCAUGCACUAUGC GCG AUUGGCUC | 1784 |
| 154 | GCCCUAUU G CUAUGCAA | 693 | UUCCAUAC UCAUG GCAUCCACUAUGC GCG AAUACCCC | 1785 |
| 159 | AUUCCUAU G CAAUCUCC | 694 | CCAGAUUC UGAUG GCAUGCACUAUGC GCG AUAGCAAU | 1786 |
| 213 | GGCCCCAC G CAAAGCUU | 695 | AAGCUUUG UGAUG GCAUGCACUAUGC GCG GUGCCGCC | 1787 |
| 228 | UUCUUCAU C AGGGAAUC | 696 | GAUUCCCU UGAUG GCAUGCACUAUGC GCG AUGAACAA | 1788 |
| 264 | CAGAUUAU G AAUCCCCU | 697 | AGCCCAUU UCAUG GCAUGCACUAUGC GCG AUAAUCUC | 1789 |
| 283 | UCCACAUU C CCCUCGGG | 698 | CCCCACCC UCAUC CCAUCCACUAUGC GCG AAUGUGGA | 1790 |
| 300 | GCUCUUCU G AUGAUCCC | 699 | CGGAUCAU UCAUC GCAUGCACUAUGC GCG AGAAGACC | 1791 |
| 303 | CUUCUCAU C AUCCCACC | 700 | GCUCCCAU UCAUC GCAUGCACUAUGC GCG AUCACAAC | 1792 |
| 322 | CCAUCUAU C CACCCAUC | 701 | CAUCCCUC UCAUG GCAUGCACUAUGC GCG AUACAUCC | 1793 |
| 336 | AUCUCUCU C ACUCUCUC | 702 | CACACACU UGAUC GCAUGCACUAUGC GCG ACACACAU | 1794 |
| 441 | CCAAAAAU C AUAAUCAA | 703 | UUCAUUAU UGAUC GCAUGCACUAUGC GCG AUUUUUCC | 1795 |
| 447 | AUCAUAAU C AAUUCAUU | 704 | AAUGAAUU UCAUG GCAUGCACUAUGC GCG AUUAUCAU | 1796 |
| 456 | AAUUCAUU C ACCCUCUU | 705 | AACACCCU UCAUC GCAUGCACUAUGC GCG AAUCAAUU | 1797 |
| 466 | CCCUCUUU C CUCCCAUU | 706 | AAUCCCAC UCAUC GCAUGCACUAUGC GCG AAACACCC | 1798 |
| 469 | UCUUUGCU C CCAUUUCU | 707 | ACAAAUCC UCAUG GCAUGCACUAUGC GCG AGCAAAGA | 1799 |
| 483 | UCUGGAAU C AUUCUUUC | 708 | GAAAGAAU UGAUC GCAUGCACUAUGC GCG AUUCCAGA | 1800 |
| 546 | GAGAGUCU G AAUUUUAU | 709 | AUAAAAUU UCAUC GCAUGCACUAUGC GCG ACACUCUC | 1801 |
| 592 | ACAACUCU C AACCACCU | 710 | AGCUCCUU UCAUC GCAUGCACUAUGC GCG ACACUUCU | 1802 |
| 610 | AUCCCUCU C ACAAAAAC | 711 | GUUUUUCU UCAUC GCAUGCACUAUGC GCG ACACCCAU | 1803 |
| 678 | UUCUCACU C AUCCUCAU | 712 | AUCACCAU UCAUC GCAUGCACUAUGC GCG ACUCACAA | 1804 |
| 681 | UCAGUCAU G CUCAUCUU | 713 | AACAUCAC UCAUC GCAUGCACUAUGC GCG AUCACUGA | 1805 |
| 684 | CUCAUCCU C AUCUUUCC | 714 | CCAAACAU UGAUC GCAUGCACUAUGC GCG ACCAUCAC | 1806 |
| 691 | UCAUCUUU C CCUUCUUC | 715 | CAACAACC UCAUC GCAUGCACUAUGC GCG AAACAUCA | 1807 |
| 727 | CCAUCCUU C ACAAUCAA | 716 | UUCAUUCU UCAUC GCAUGCACUAUGC GCG AACCAUGC | 1808 |
| 733 | UUCACAAU C AAUCCAAA | 717 | UUUCCAUU UCAUC GCAUGCACUAUGC GCG AUUCUCAA | 1809 |
| 749 | AACAACCU C CUCCACAC | 718 | CUCUCCAC UCAUC GCAUGCACUAUGC GCG ACCUUCUU | 1810 |
| 811 | ACACUAUU C AAAUAAAA | 719 | UUUUAUUU UCAUC GCAUGCACUAUGC GCG AAUACUCU | 1811 |
| 841 | CCCUAACU C AAACAUCU | 720 | ACAUCUUU UCAUC GCAUGCACUAUGC GCG ACUUACCC | 1812 |
| 865 | CAAAGAAU C AACAACAC | 721 | CUCUUCUU UGAUC GCAUGCACUAUGC GCG AUUCUUUC | 1813 |
| 877 | AACACAUU C AAAUUAUU | 722 | AAUAAUUU UCAUC GCAUGCACUAUGC GCG AAUCUCUU | 1814 |
| 921 | ACACAGAC C AACUUUCC | 723 | CCAAAGUU UCAUC GCAUGCACUAUGC GCG CUCUCUCU | 1815 |
| 970 | UACAAAAU C ACACCUCU | 724 | ACACCUCU UCAUC GCAUGCACUAUGC GCG AUUUUCUA | 1816 |
| 987 | CCUUAACU C AUUUCUUC | 725 | CAACAAAU UCAUC GCAUGCACUAUGC GCG ACUUAACC | 1817 |
| 1048 | ACACACAU C CUCACUUU | 726 | AAACUCAC UCAUC GCAUGCACUAUGC GCG AUCUCUCU | 1818 |
| 1051 | CACAUCCU C ACUUUCAU | 727 | AUCAAACU UCAUC GCAUGCACUAUGC GCG ACCAUCUC | 1819 |
| 1065 | CAUUUCUU C ACCUACUC | 728 | CACUACCU UCAUC GCAUGCACUAUGC GCG AACAAAUC | 1820 |
| 1075 | CCUACUCU C CACAUACC | 729 | CCUAUCUC UCAUC GCAUGCACUAUGC GCG ACACUACC | 1821 |
| 1083 | CCACAUAC C CACCACAU | 730 | AUCUGGUC UCAUC GCAUGCACUAUGC GCG CUAUCUCC | 1822 |
| 1107 | UCCCCUUU C CAUCCACU | 731 | ACUCCAUC UCAUC GCAUGCACUAUGC GCG AAACCCCA | 1823 |
| 1116 | CAUGGAGU G ACCAUAGC | 732 | GCUAUGCU UGAUG GCAUGCACUAUGC GCG ACUCCAUG | 1824 |
| 1142 | CUUACAUU G AAUGUAGA | 733 | UCUACAUU UGAUG GCAUGCACUAUGC GCG AAUGUAAG | 1825 |
| 1184 | GUUGUCAC G CUUCUUCU | 734 | AGAAGAAG UGAUG GCAUGCACUAUGC GCG GUGACAAC | 1826 |
| 1196 | CUUCUUUU G AGCAACUU | 735 | AAGUUGCU UGAUG GCAUGCACUAUGC GCG AAAAGAAG | 1827 |
| 1214 | CUUACACU G AAGAAAGG | 736 | CCUUUCUU UGAUG GCAUGCACUAUGC GCG AGUGUAAG | 1828 |
| 1229 | GGCAGAAU G AGUGCUUC | 737 | GAAGCACU UGAUG GCAUGCACUAUGC GCG AUUCUGCC | 1829 |
| 1233 | GAAUGAGU G CUUCAGAA | 738 | UUCUGAAG UGAUG GCAUGCACUAUGC GCG ACUCAUUC | 1830 |
| 1245 | CAGAAUGU G AUUUCCUA | 739 | UAGGAAAU UGAUG GCAUGCACUAUGC GCG ACAUUCUG | 1831 |
| 1330 | GGGAGACU G CACCUGAU | 740 | AUCAGGUG UGAUG GCAUGCACUAUGC GCG AGUCUCCC | 1832 |
| 1336 | CUGCACCU G AUGGAAAA | 741 | UUUUCCAU UGAUG GCAUGCACUAUGC GCG AGGUGCAG | 1833 |
| 1352 | AGAUAUAU G ACUGCUUC | 742 | GAAGCAGU UGAUG GCAUGCACUAUGC GCG AUAUAUCU | 1834 |
| 1356 | AUAUGACU G CUCCAUGA | 743 | UCAUGAAG UGAUG GCAUGCACUAUGC GCG AGUCAUAU | 1835 |
| 1363 | UGCUUCAU G ACAUUCCU | 744 | AGGAAUGU UGAUG GCAUGCACUAUGC GCG AUGAAGCA | 1836 |
| 1422 | UCCCUUUU G CAUCAUUG | 745 | CAAUGAUG UGAUG GCAUGCACUAUGC GCG AAAAGGGA | 1837 |
| 1442 | UUAAGGAU G AUAAAAAA | 746 | UUUUUUAU UGAUG GCAUGCACUAUGC GCG AUCCUUAA | 1838 |
| 1505 | ACAGGCCC G ACAUUGUG | 747 | CACAAUGU UGAUG GCAUGCACUAUGC GCG AGCCCUGU | 1839 |
| 1542 | CACCCCAU G AGGGAAGC | 748 | GCUUCCCU UGAUG GCAUGCACUAUGC GCG AUGGCGUG | 1840 |

Input Sequence = HSCD20A. Cut Site = YG/M or UG/U.
Stem Length = 8. Core Sequence = UGAUG GCAUGCACUAUGC GCG
HSCD2CA (Human mRNA for 0D2C receptor (S7); 1597 bp)

TABLE VI
Human CD20 Zinzyme Ribozyme and Substrate Sequence

| | | | | Rz Seq |
|---|---|---|---|---|
| Pos | Substrate | Seq ID | Ribozyme | ID |
| 9 | AACAAACU G CACCCACU | 685 | AGUGGGUG GCCGAAAGGCGAGUCAAGGUCU AGUUUGUU | 1841 |
| 25 | UGAACUCC G CAGCUAGC | 687 | GCUAGCUG GCCGAAAGGCGAGUCAAGGUCU GGAGUUCA | 1842 |
| 28 | ACUCCGCA G CUAGCAUC | 749 | GAUGCUAG GCCGAAAGGCGAGUCAAGGUCU UGCGGAGU | 1843 |
| 32 | CGCAGCUA G CAUCCAAA | 750 | UUUGGAUG GCCGAAAGGCGAGUCAAGGUCU UAGCUGCG | 1844 |
| 44 | CCAAAUCA G CCCUUGAG | 751 | CUCAAGGG GCCGAAAGGCGAGUCAAGGUCU UGAUUUGG | 1845 |
| 60 | GAUUUGAG G CCUUGGAG | 752 | CUCCAAGG GCCGAAAGGCGAGUCAAGGUCU CUCAAAUC | 1846 |
| 77 | ACUCAGGA G UUUUGAGA | 753 | UCUCAAAA GCCGAAAGGCGAGUCAAGGUCU UCCUGAGU | 1847 |
| 86 | UUUUGAGA G CAAAAUGA | 754 | UCAUUUUG GCCGAAAGGCGAGUCAAGGUCU UCUCAAAA | 1848 |
| 112 | GAAAUUCA G UAAAUGGG | 755 | CCCAUUUA GCCGAAAGGCGAGUCAAGGUCU UGAAUUUC | 1849 |
| 130 | CUUUCCUG G CAGAGCCA | 756 | UGGCUCUG GCCGAAAGGCGAGUCAAGGUCU CAGGAAAG | 1850 |
| 135 | CUGGCAGA G CCAAUGAA | 757 | UUCAUUGG GCCGAAAGGCGAGUCAAGGUCU UCUGCCAG | 1851 |
| 146 | AAUGAAAG G CCCUAUUG | 758 | CAAUAGGG GCCGAAAGGCGAGUCAAGGUCU CUUUCAUU | 1852 |
| 154 | GCCCUAUU G CUAUGCAA | 693 | UUGCAUAG GCCGAAAGGCGAGUCAAGGUCU AAUAGGGC | 1853 |
| 159 | AUUGCUAU G CAAUCUGG | 694 | CCAGAUUG GCCGAAAGGCGAGUCAAGGUCU AUAGCAAU | 1854 |
| 167 | GCAAUCUG G UCCAAAAC | 759 | GUUUUGGA GCCGAAAGGCGAGUCAAGGUCU CAGAUUGC | 1855 |
| 192 | AGGAGGAU G UCUUCACU | 760 | AGUGAAGA GCCGAAAGGCGAGUCAAGGUCU AUCCUCCU | 1856 |
| 202 | CUUCACUG G UGGGCCCC | 761 | GGGGCCCA GCCGAAAGGCGAGUCAAGGUCU CAGUGAAG | 1857 |
| 206 | ACUGGUGG G CCCCACGC | 762 | GCGUGGGG GCCGAAAGGCGAGUCAAGGUCU CCACCAGU | 1858 |
| 213 | GGCCCCAC G CAAAGCUU | 695 | AAGCUUUG GCCGAAAGGCGAGUCAAGGUCU GUGGGGCC | 1859 |
| 218 | CACGCAAA G CUUCUUCA | 763 | UGAAGAAG GCCGAAAGGCGAGUCAAGGUCU UUUGCGUG | 1860 |
| 250 | CUUUGGGG G CUGUCCAG | 764 | CUGGACAG GCCGAAAGGCGAGUCAAGGUCU CCCCAAAG | 1861 |
| 253 | UGGGGGCU G UCCAGAUU | 765 | AAUCUGGA GCCGAAAGGCGAGUCAAGGUCU AGCCCCCA | 1862 |
| 270 | AUGAAUGG G CUCUUCCA | 766 | UGGAAGAG GCCGAAAGGCGAGUCAAGGUCU CCAUUCAU | 1863 |
| 283 | UCCACAUU G CCCUGCGG | 698 | CCCCAGGG GCCGAAAGGCGAGUCAAGGUCU AAUGUGGA | 1864 |
| 293 | CCUGGGGG G UCUUCUGA | 767 | UCAGAAGA GCCGAAAGGCGAGUCAAGGUCU CCCCCAGG | 1865 |
| 310 | UGAUCCCA G CAGGGAUC | 768 | GAUCCCUG GCCGAAAGGCGAGUCAAGGUCU UGUGAUCA | 1866 |
| 322 | GGAUCUAU G CACCCAUC | 701 | GAUGGGUG GCCGAAAGGCGAGUCAAGGUCU AUAGAUCC | 1867 |
| 332 | ACCCAUCU G UGUGACUG | 769 | CAGUCACA GCCGAAAGGCGAGUCAAGGUCU AGAUGGGU | 1868 |
| 334 | CCAUCUGU G UGACUCUG | 770 | CACAGUCA GCCGAAAGGCGAGUCAAGGUCU ACAGAUGG | 1869 |
| 340 | GUGUGACU G UGUGGUAC | 771 | GUACCACA GCCGAAAGGCGAGUCAAGGUCU AGUCACAC | 1870 |
| 342 | GUGACUGU G UGGUACCC | 772 | GGGUACCA GCCGAAAGGCGAGUCAAGGUCU ACAGUCAC | 1871 |
| 345 | ACUGUGUG G UACCCUCU | 773 | AGAGGGUA GCCGAAAGGCGAGUCAAGGUCU CACACAGU | 1872 |
| 362 | CUGGGGAG G CAUUAUGU | 774 | ACAUAAUG GCCGAAAGGCGAGUCAAGGUCU CUCCCCAG | 1873 |
| 369 | GGCAUUAU G UAUAUUAU | 775 | AUAAUAUA GCCGAAAGGCGAGUCAAGGUCU AUAAUGCC | 1874 |
| 394 | CACUCCUG G CAGCAACG | 776 | CCUUGCUG GCCGAAAGGCGAGUCAAGGUCU CAGGAGUG | 1875 |
| 397 | UCCUCCCA G CAACGGAC | 777 | CUCCGUUG GCCGAAAGGCGAGUCAAGGUCU UGCCAGGA | 1876 |
| 420 | UCCAGGAA G UGUUUCGU | 778 | ACCAAACA GCCGAAAGGCGAGUCAAGGUCU UUCCUGGA | 1877 |
| 422 | CAGGAAGU G UUUGGUCA | 779 | UGACCAAA GCCGAAAGGCGAGUCAAGGUCU ACUUCCUG | 1878 |
| 427 | AGUGUUUG G UCAAAGGA | 780 | UCCUUUCA GCCGAAAGGCGAGUCAAGGUCU CAAACACU | 1879 |
| 458 | UUCAUUCA G CCUCUUUC | 781 | CAAAGAGC GCCGAAAGGCGAGUCAAGGUCU UCAAUGAA | 1880 |
| 466 | CCCUCUUU G CUCCCAUU | 706 | AAUCCCAC GCCGAAAGGCGAGUCAAGGUCU AAACAGGC | 1881 |
| 469 | UCUUUGCU G CCAUUUCU | 707 | AGAAAUGG GCCGAAAGGCGAGUCAAGGUCU AGCAAAGA | 1882 |
| 542 | AAUGGACA G UCUCAAUU | 782 | AAUUCAGA GCCGAAAGGCGAGUCAAGGUCU UCUCCAUU | 1883 |
| 559 | UUAUUACA G CUCACACA | 783 | UGUCUGAC GCCGAAAGGCGAGUCAAGGUCU UCUAAUAA | 1884 |
| 590 | AUACAACU G UGAACCAG | 784 | CUCCUUCA GCCGAAAGGCGAGUCAAGGUCU AGUUGUAU | 1885 |
| 598 | GUCAACCA G CUAAUCCC | 785 | CCCAUUAC GCCGAAAGGCGAGUCAAGGUCU UCCUUCAC | 1886 |
| 638 | CCAAUACU G UUACAGCA | 786 | UCCUCUAA GCCGAAAGGCGAGUCAAGGUCU ACUAUUGC | 1887 |
| 644 | CUGUUACA G CAUACAAU | 787 | AUUGUAUG GCCGAAAGGCGAGUCAAGGUCU UGUAACAG | 1888 |
| 657 | CAAUCUCU G UUCUUGGG | 788 | CCCAAGAA GCCGAAAGGCGAGUCAAGGUCU AGAGAUUG | 1889 |
| 665 | GUUCUUGG G CAUUUUGU | 789 | ACAAAAUG GCCGAAAGGCGAGUCAAGGUCU CCAAGAAC | 1890 |
| 672 | GGCAUUUU G UCAGUGAU | 790 | AUCACUGA GCCGAAAGGCGAGUCAAGGUCU AAAAUGCC | 1891 |
| 676 | UUUUGUCA G UGAUCCUG | 791 | CACCAUCA GCCGAAAGGCGAGUCAAGGUCU UGACAAAA | 1892 |
| 681 | UCAGUGAU G CUGAUCUC | 713 | AAGAUCAG GCCGAAAGGCGAGUCAAGGUCU AUCACUGA | 1893 |
| 691 | UGAUCUUU G CCUUCUUC | 715 | GAAGAAGG GCCGAAAGGCGAGUCAAGGUCU AAAGAUCA | 1894 |
| 709 | AGGAACUU G UAAUAGCU | 792 | AGCUAUUA GCCGAAAGGCGAGUCAAGGUCU AAGUUCCU | 1895 |
| 715 | UUGUAAUA G CUGGCAUC | 793 | GAUGCCAG GCCGAAAGGCGAGUCAAGGUCU UAUUACAA | 1896 |
| 719 | AAUAGCUG G CAUCGUUG | 794 | CAACGAUG GCCGAAAGGCGAGUCAAGGUCU CAGCUAUU | 1897 |
| 724 | CUGUCAUC G UUCAGAAU | 795 | AUUCUCAA GCCGAAAGGCGAGUCAAGGUCU GAUGCCAG | 1898 |
| 747 | AAAACAAC G UCCUCCAC | 796 | CUGGAGCA GCCGAAAGGCGAGUCAAGGUCU GUUCUUUU | 1899 |
| 749 | AAGAACGU G CUCCAGAC | 718 | GUCUGGAG GCCGAAAGGCGAGUCAAGGUCU ACGUUCUU | 1900 |
| 772 | CUAACAUA G UUCUCCUG | 797 | CAGGAGAA GCCGAAAGGCGAGUCAAGGUCU UAUGUUAG | 1901 |
| 780 | GUUCUCCU G UCACCAGA | 798 | UCUCCUGA GCCGAAAGGCGAGUCAAGGUCU ACCAGAAC | 1902 |
| 784 | UCCUCUCA G CAGAAGAA | 799 | UUCUUCUG GCCGAAAGGCGAGUCAAGGUCU UGACAGGA | 1903 |
| 826 | AACAACAA G UCCUUGGG | 800 | CCCAACCA GCCGAAAGGCGAGUCAAGGUCU UUCUUCUU | 1904 |
| 829 | AAGAACUC G UUGGGCUA | 801 | UAGCCCAA GCCGAAAGGCGAGUCAAGGUCU CACUUCUU | 1905 |
| 834 | GUGCUUGG G CUAACUGA | 802 | UCAGUCAG GCCGAAAGGCGAGUCAAGGUCU CCAACCAC | 1906 |
| 974 | AAAUGACA G CUCUCCUU | 803 | AAGGAGAG GCCGAAAGGCGAGUCAAGGUCU UGUCAUUU | 1907 |
| 985 | CUCCUUAA G UGAUUUCU | 804 | AGAAAUCA GCCGAAAGGCGAGUCAAGGUCU UUAAGCAG | 1908 |
| 997 | UUUCUUCU G UUUUCUGU | 805 | ACAGAAAA GCCGAAAGGCGAGUCAAGGUCU AGAAGAAA | 1909 |
| 1004 | UCUUUUCU G UUUCCUUU | 806 | AAAGGAAA GCCGAAAGGCGAGUCAAGGUCU AGAAAACA | 1910 |
| 1024 | AAACAUUA G UGUUCAUA | 807 | UAUGAACA GCCGAAAGGCGAGUCAAGGUCU UAAUCUUU | 1911 |
| 1026 | ACAUUACU G UUCAUAGC | 808 | CCUAUCAA GCCGAAAGGCGAGUCAAGGUCU ACUAAUGU | 1912 |
| 1033 | UCUUCAUA G CUUCCAAG | 809 | CUUCGAAG GCCGAAAGGCGAGUCAAGGUCU UAUCAACA | 1913 |
| 1048 | ACACACAU G CUCACUUU | 726 | AAACUCAC GCCGAAAGGCGAGUCAAGGUCU AUCUCUCU | 1914 |
| 1068 | UUCUUCAC G UACUCUCC | 810 | CCACACUA GCCGAAAGGCGAGUCAAGGUCU CUCAACAA | 1915 |
| 1075 | CCUACUCU G CACAUACC | 729 | CCUAUCUG GCCGAAAGGCGAGUCAAGGUCU ACAGUACC | 1916 |
| 1083 | GCACAUAC G CACCACAU | 730 | AUCUCCUC GCCGAAAGGCGAGUCAAGGUCU CUAUCUGC | 1917 |
| 1101 | UCUAUCUG G CCUUUGCA | 811 | UCCAAAGG GCCGAAAGGCGAGUCAAGGUCU CAGAUAGA | 1918 |
| 1107 | UCGCCUUU G CAUGGACU | 731 | ACUCCAUC GCCGAAAGGCGAGUCAAGGUCU AAAGGCCA | 1919 |
| 1114 | UCCAUCCA G UCACCAUA | 812 | UAUCCUCA GCCGAAAGGCGAGUCAAGGUCU UCCAUCCA | 1920 |
| 1123 | UCACCAUA G CUCCUUCU | 813 | ACAACCAC GCCGAAAGGCGAGUCAAGGUCU UAUCCUCA | 1921 |
| 1146 | CAUUCAAU G UACACAAU | 814 | AUUCUCUA GCCGAAAGGCGAGUCAAGGUCU AUUCAAUC | 1922 |
| 1155 | UACACAAU G UACCCAUU | 815 | AAUCCCUA GCCGAAAGGCGAGUCAAGGUCU AUUCUCUA | 1923 |
| 1158 | ACAAUCUA G CCAUUGUA | 816 | UACAAUCG GCCGAAAGGCGAGUCAAGGUCU UACAUUCU | 1924 |
| 1164 | UACCCAUU G UAGCACCU | 817 | ACCUCCUA GCCGAAAGGCGAGUCAAGGUCU AAUCGCUA | 1925 |
| 1167 | CCAUUCUA G CACCUUCU | 818 | ACAACCUC GCCGAAAGGCGAGUCAAGGUCU UACAAUCC | 1926 |
| 1170 | UCCUACCA G CUUCUCUU | 819 | AACACAAC GCCGAAAGGCGAGUCAAGGUCU UCCUACAA | 1927 |
| 1174 | ACCACCUU G UCUCCUCA | 820 | UCACAACA GCCGAAAGGCGAGUCAAGGUCU AACCUCCU | 1928 |
| 1176 | CACCUUCU G UUCUCACC | 821 | CCUCACAA GCCGAAAGGCGAGUCAAGGUCU ACAACCUC | 1929 |
| 1179 | CUUCUCUU G UCACGCUU | 822 | AACCCUCA GCCGAAAGGCGAGUCAAGGUCU AACACAAC | 1930 |
| 1184 | CUUCUCAC G CUUCUUCU | 734 | AGAAGAAC GCCGAAAGGCGAGUCAAGGUCU CUCACAAC | 1931 |
| 1198 | UCUUUUGA G CAACUUUC | 823 | GAAAGUUG GCCGAAAGGCGAGUCAAGGUCU UCAAAAGA | 1932 |
| 1222 | CAACAAAC G CACAAUCA | 824 | UCAUUCUC GCCGAAAGGCGAGUCAAGGUCU CUUUCUUC | 1933 |
| 1231 | CACAAUCA G UCCUUCAC | 825 | CUCAACCA GCCGAAAGGCGAGUCAAGGUCU UCAUUCUG | 1934 |
| 1233 | CAAUCACU G CUUCACAA | 738 | UUCUCAAC GCCGAAAGGCGAGUCAAGGUCU ACUCAUUC | 1935 |
| 1243 | UUCACAAU G UCAUUUCC | 826 | CCAAAUCA GCCGAAAGGCGAGUCAAGGUCU AUUCUCAA | 1936 |
| 1261 | ACUAACCU G UUCCUUCC | 827 | CCAACCAA GCCGAAAGGCGAGUCAAGGUCU ACGUCACU | 1937 |
| 1274 | UUCCAUAC G CUUUUUAG | 828 | CUAAAAAC GCCGAAAGGCGAGUCAAGGUCU CUAUCCAA | 1938 |
| 1282 | GCUUUUUA G UAUAGUAU | 829 | AUACUAUA GCCGAAAGGCGAGUCAAGGUCU UAAAAAGC | 1939 |
| 1287 | UUAGUAUA G UAUUUUUU | 830 | AAAAAAUA GCCGAAAGGCGAGUCAAGGUCU UAUACUAA | 1940 |
| 1299 | UUUUUUUU G UCAUUUUC | 831 | GAAAAUGA GCCGAAAGGCGAGUCAAGGUCU AAAAAAAA | 1941 |
| 1315 | CUCCAUCA G CAACCAGG | 832 | CCUGGUUG GCCGAAAGGCGAGUCAAGGUCU UGAUGGAG | 1942 |
| 1330 | GGGAGACU G CACCUGAU | 740 | AUCAGGUG GCCGAAAGGCGAGUCAAGGUCU AGUCUCCC | 1943 |
| 1356 | AUAUGACU G CUCCAUGA | 743 | UCAUGAAG GCCGAAAGGCGAGUCAAGGUCU AGUCAUAU | 1944 |
| 1401 | ACAUCUAC G UUUUUGGU | 833 | ACCAAAAA GCCGAAAGGCGAGUCAAGGUCU GUAGAUGU | 1945 |
| 1408 | CGUUUUUG G UGGAGUCC | 834 | GUACUCCA GCCGAAAGGCGAGUCAAGGUCU CAAAAACG | 1946 |
| 1413 | UUGGUGGA G UCCCUUUU | 835 | AAAAGGGA GCCGAAAGGCGAGUCAAGGUCU UCCACCAA | 1947 |
| 1422 | UCCCUUUC G CAUCAUUG | 745 | CAAUGAUG GCCGAAAGGCGAGUCAAGGUCU AAAAGGGA | 1948 |
| 1430 | GCAUCAUU G UUUUAAGG | 836 | CCUUAAAA GCCGAAAGGCGAGUCAAGGUCU AAUGAUGC | 1949 |
| 1502 | UCUACAGG G CUGACAUC | 837 | AAUGUCAG GCCGAAAGGCGAGUCAAGGUCU CCUGUAGA | 1950 |
| 1511 | CUGACAUU G UGGCACAU | 838 | AUGUCCCA GCCGAAAGGCGAGUCAAGGUCU AAUGUCAG | 1951 |
| 1514 | ACAUUGUG G CACAUUCU | 839 | AGAAUGUG GCCGAAAGGCGAGUCAAGGUCU CACAAUGU | 1952 |
| 1527 | UUCUUAGA G UUACCACA | 840 | UGUGGUAA GCCGAAAGGCGAGUCAAGGUCU UCUAAGAA | 1953 |
| 1549 | UGAGGGAA G CUCUAAAU | 841 | AUUUAGAG GCCGAAAGGCGAGUCAAGGUCU UUCCCUCA | 1954 |
| 1559 | UCUAAAUA G CCAACACC | 842 | GGUGUUGG GCCGAAAGGCGAGUCAAGGUCU UAUUUAGA | 1955 |
| 1573 | ACCCAUCU G UUUUUUGU | 843 | ACAAAAAA GCCGAAAGGCGAGUCAAGGUCU AGAUGGGU | 1956 |
| 1580 | UGUUUUUU G UAAAAACA | 844 | UGUUUUUA GCCGAAAGGCGAGUCAAGGUCU AAAAAACA | 1957 |
| 1589 | UAAAAACA G CAUAGCUU | 845 | AAGCUAUG GCCGAAAGGCGAGUCAAGGUCU UGUUUUUA | 1958 |

Input Sequence = HSCD20A. Cut Site = G/Y
Stem Length = 8. Core Sequence = GCcgaaagGCGaGuCaaGGuCu
HSCD20A (Human mRNA for CD20 receptor (S7); 1597 bp)

TABLE VII
Human CD20 DNAzyme and Substrate Sequence

| Pos | Substrate | Seq ID | DNAzyme | Seq ID |
|---|---|---|---|---|
| 9 | AACAAACU G CACCCACU | 685 | AGIGGGIG GGCTAGCTACAACGA AGTTIGTT | 1959 |
| 11 | CAAACUGC A CCCACUGA | 349 | TCAGIGGG GGCTAGCTACAACGA GCAGTTIG | 1960 |
| 15 | CUGCACCC A CUGAACUC | 352 | GAGTICAG GGCTAGCTACAACGA GGGIGCAG | 1961 |
| 20 | CCCACUGA A CUCCGCAG | 846 | CTGCGGAG GGCTAGCTACAACGA TCAGTGGG | 1962 |
| 25 | UGAACUCC G CAGCUAGC | 687 | GCTAGCTG GGCTAGCTACAACGA GGAGTTCA | 1963 |
| 28 | ACUCCGCA C CUACCAUC | 749 | GATGCTAG GGCTAGCTACAACGA TGCGCAGT | 1964 |
| 32 | CGCAGCUA C CAUCCAAA | 750 | TTTGGATG GGCTAGCTACAACGA TAGCTGCG | 1965 |
| 34 | CAGCUAGC A UCCAAAUC | 358 | GATTTGGA GGCTAGCTACAACGA GCTAGCTG | 1966 |
| 40 | GCAUCCAA A UCAGCCCU | 847 | AGGGCTGA GGCTAGCTACAACGA TTGGATGC | 1967 |
| 44 | CCAAAUCA G CCCUUGAG | 751 | CTCAAGGG GGCTAGCTACAACGA TGATTTGC | 1968 |
| 53 | CCCUUGAG A UUUGAGGC | 848 | GCCTCAAA GGCTACCTACAACGA CTCAACGG | 1969 |
| 60 | GAUUUGAC G CCUUGGAC | 752 | CTCCAAGG GGCTAGCTACAACGA CTCAAATC | 1970 |
| 69 | CCUUCCAG A CUCAGGAG | 849 | CTCCTGAG GGCTAGCTACAACGA CTCCAAGG | 1971 |
| 77 | ACUCAGGA G UUUUGAGA | 753 | TCTCAAAA GGCTAGCTACAACGA TCCTGACT | 1972 |
| 86 | UUUUGAGA G CAAAAUGA | 754 | TCATTTTG GGCTAGCTACAACGA TCTCAAAA | 1973 |
| 91 | ACACCAAA A UGACAACA | 850 | TGTTGTCA GGCTAGCTACAACGA TTTGCTCT | 1974 |
| 94 | CCAAAAUG A CAACACCC | 851 | GGCTGTTG GGCTACCTACAACGA CATTTTCC | 1975 |
| 97 | AAAUGACA A CACCCAGA | 852 | TCTCCCTC GGCTACCTACAACGA TGTCATTT | 1976 |
| 99 | AUCACAAC A CCCACAAA | 371 | TTTCTGGG GGCTAGCTACAACGA GTTGTCAT | 1977 |
| 107 | ACCCAGAA A UUCAGUAA | 853 | TTACTGAA GGCTAGCTACAACGA TTCTGGGT | 1978 |
| 112 | GAAAUUCA G UAAAUGGG | 755 | CCCATTTA GGCTAGCTACAACGA TCAATTTC | 1979 |
| 116 | UUCAGUAA A UGGGACUU | 854 | AACTCCCA GGCTAGCTACAACGA TTACTGAA | 1980 |
| 121 | UAAAUCGC A CUUUCCUC | 855 | CAGGAAAG GGCTACCTACAACGA CCCATTTA | 1981 |
| 130 | CUUUCCUC G CACAGCCA | 756 | TCCCTCTG GGCTACCTACAACGA CAGGAAAG | 1982 |
| 135 | CUCCCACA G CCAAUCAA | 757 | TTCATTGG GGCTACCTACAACGA TCTGCCAG | 1983 |
| 139 | CACAGCCA A UCAAAGGC | 856 | CCCTTTCA GGCTAGCTACAACGA TGCCTCTG | 1984 |
| 146 | AAUGAAAG G CCCUAUUG | 758 | CAATAGGG GGCTAGCTACAACGA CTTTCATT | 1985 |
| 151 | AAGGCCCU A UUGCUAUG | 19 | CATAGCAA GGCTACCTACAACGA ACCGCCTT | 1986 |
| 154 | GCCCUAUU G CUAUGCAA | 693 | TTGCATAC GGCTACCTACAACGA AATAGGCC | 1987 |
| 157 | CUAUUCCU A UGCAAUCU | 21 | ACATTCCA GGCTACCTACAACGA AGCAATAC | 1988 |
| 159 | AUUGCUAU G CAAUCUCC | 694 | CCACATTC GGCTACCTACAACGA ATAGCAAT | 1989 |
| 162 | GCUAUGCA A UCUGGUCC | 857 | GGACCAGA GGCTAGCTACAACGA TGCATAGC | 1990 |
| 167 | CCAAUCUC C UCCAAAAC | 759 | CTTTTGGA GGCTAGCTACAACGA CAGATTGC | 1991 |
| 174 | GGUCCAAA A CCACUCUU | 858 | AAGACTCC GGCTACCTACAACGA TTTCCACC | 1992 |
| 177 | CCAAAACC A CUCCUCAC | 391 | CTCAACAG GGCTACCTACAACGA CCTTTTCC | 1993 |
| 190 | UCACCACC A UCUCCUCA | 859 | TCAACACA GGCTACCTACAACGA CCTCCTCA | 1994 |
| 192 | ACCACCAU C UCUUCACU | 760 | ACTCAACA GGCTACCTACAACGA ATCCTCCT | 1995 |
| 198 | AUCUCUUC A CUCGUCGG | 396 | CCCACCAG GGCTAGCTACAACGA GAACACAT | 1996 |
| 202 | CUUCACUC G UGCCCCCC | 761 | CCGCCCCA GGCTACCTACAACGA CAGTCAAC | 1997 |
| 206 | ACUCCUCC C CCCCACCC | 762 | CCCTCCGG GGCTAGCTACAACGA CCACCACT | 1998 |
| 211 | UCCCCCCC A CCCAAACC | 401 | GCTTTCCG GGCTACCTACAACGA CCCGCCCA | 1999 |
| 213 | CCCCCCAC C CAAAGCUU | 695 | AACCTTTC GGCTACCTACAACGA CTCCGCCC | 2000 |
| 218 | CACCCAAA C CUUCUUCA | 763 | TGAAGAAG GGCTACCTACAACGA TTTCCCTC | 2001 |
| 226 | CCUUCUUC A UCAGGGAA | 405 | TTCCCTCA GGCTAGCTACAACGA GAACAAGC | 2002 |
| 234 | AUCACGGA A UCUAAGAC | 860 | CTCTTACA GGCTAGCTACAACGA TCCCTCAT | 2003 |
| 241 | AAUCUAAC A CUUUCCCC | 961 | CCCCAAAC GGCTACCTACAACGA CTTACATT | 2004 |
| 250 | CUUUCCCC G CUCUCCAC | 764 | CTCGACAC GGCTACCTACAACGA CCCCAAAC | 2005 |
| 253 | UCCCCCCU C UCCACAUU | 765 | AATCTCCA GGCTACCTACAACGA AGCCCCCA | 2006 |
| 259 | CUGUCCAG A UUAUGAAU | 862 | ATTCATAA GGCTAGCTACAACGA CTGGACAG | 2007 |
| 262 | UCCAGAUU A UGAAUGGG | 40 | CCCATTCA GGCTAGCTACAACGA AATCTGGA | 2008 |
| 266 | GAUUAUGA A UGGGCUCU | 863 | AGAGCCCA GGCTAGCTACAACGA TCATAATC | 2009 |
| 270 | AUGAAUGG G CUCUUCCA | 766 | TGGAAGAG GGCTACCTACAACGA CCATTCAT | 2010 |
| 278 | GCUCUUCC A CAUUGCCC | 414 | GGGCAATC GGCTAGCTACAACGA GGAAGAGC | 2011 |
| 280 | UCUUCCAC A UUCCCCUG | 415 | CAGGGCAA GGCTAGCTACAACGA GTGGAAGA | 2012 |
| 283 | UCCACAUC G CCCUGGGG | 698 | CCCCAGGG GGCTAGCTACAACGA AATGTGGA | 2013 |
| 293 | CCUGGGGG G UCUUCUGA | 767 | TCAGAAGA GGCTAGCTACAACGA CCCCCAGG | 2014 |
| 301 | GUCCUCUG A UGAUCCCA | 864 | TGGGATCA GGCTAGCTACAACGA CAGAAGAC | 2015 |
| 304 | UUCUGAUG A UCCCAGCA | 866 | TGCTGGGA GGCTAGCTACAACGA CATCAGAA | 2016 |
| 310 | UGAUCCCA G CAGGCAUC | 768 | GATCCCTG GGCTAGCTACAACGA TGGGATCA | 2017 |
| 316 | CAGCAGGG A UCUAUGCA | 866 | TCCATAGA GGCTAGCTACAACGA CCCTGCTG | 2018 |
| 320 | AGGGAUCU A UGCACCCA | 50 | TGGGTGCA GGCTAGCTACAACGA AGATCCCT | 2019 |
| 322 | GGAUCUAU G CACCCAUC | 701 | GATGGGTG GGCTAGCTACAACGA ATAGATCC | 2020 |
| 324 | AUCUAUGC A CCCAUCUG | 426 | CAGATGGG GGCTAGCTACAACGA GCATAGAT | 2021 |
| 328 | AUCCACCC A UCUGUGUG | 429 | CACACAGA GGCTAGCTACAACGA GGGTGCAT | 2022 |
| 332 | ACCCAUCU G UGUGACUG | 769 | CAGTCACA GGCTAGCTACAACGA AGATCGCT | 2023 |
| 334 | CCAUCUGU G UGACUGUG | 770 | CACAGTCA GGCTAGCTACAACGA ACAGATGG | 2024 |
| 337 | UCUGUGUG A CUGUGUGC | 867 | CCACACAG GGCTAGCTACAACGA CACACAGA | 2025 |
| 340 | GUGUGACU G UGUGGUAC | 771 | GTACCACA GGCTAGCTACAACGA AGTCACAC | 2026 |
| 342 | GUGACUGU G UGGUACCC | 772 | GGGTACCA GGCTAGCTACAACGA ACAGTCAC | 2027 |
| 345 | ACUGUGUG G UACCCUCU | 773 | AGAGGGTA GGCTAGCTACAACGA CACACAGT | 2028 |
| 347 | UGUGUGGU A CCCUCUCU | 52 | AGAGAGGC GGCTAGCTACAACGA ACCACACA | 2029 |
| 362 | CUGGCGAG G CAUUAUGU | 774 | ACATAATG GGCTAGCTACAACGA CTCCCCAG | 2030 |
| 364 | GGGGAGGC A UUAUGUAU | 437 | ATACATAA GGCTAGCTACAACGA GCCTCCCC | 2031 |
| 367 | GAGGCAUU A UGUAUAUU | 56 | AATATACA GGCTAGCTACAACGA AATGCCTC | 2032 |
| 369 | GGCAUUAU G UAUAUUAU | 775 | ATAATATA GGCTAGCTACAACGA ATAATGCC | 2033 |
| 371 | CAUUAUGU A UAUUAUUU | 57 | AAATAATA GGCTAGCTACAACGA ACATAATG | 2034 |
| 373 | UUAUGUAU A UUAUUUCC | 58 | GGAAATAA GGCTAGCTACAACGA ATACATAA | 2035 |
| 376 | UGUAUAUU A UUUCCGGA | 60 | TCCGGAAA GGCTAGCTACAACGA AATATACA | 2036 |
| 384 | AUUUCCCG A UCACUCCU | 868 | AGGAGTGA GGCTAGCTACAACGA CCGGAAAT | 2037 |
| 387 | UCCGGAUC A CUCCUGGC | 439 | GCCAGGAG GGCTAGCTACAACGA GATCCGGA | 2038 |
| 394 | CACUCCUG G CAGCAACG | 776 | CCTTCCTC GGCTAGCTACAACGA CAGGAGTG | 2039 |
| 397 | UCCUGGCA G CAACGGAG | 777 | CTCCGTTG GGCTAGCTACAACGA TCCCAGGA | 2040 |
| 400 | UGGCAGCA A CGGAGAAA | 369 | TTTCTCCG GGCTAGCTACAACGA TCCTCCCA | 2041 |
| 410 | GGAGAAAA A CUCCAGGA | 870 | TCCTGGAG GGCTAGCTACAACGA TTTTCTCC | 2042 |
| 420 | UCCAGGAA G UGUUUGGU | 778 | ACCAAACA GGCTAGCTACAACGA TTCCTGGA | 2043 |
| 422 | CAGGAAGU G UUUGGUCA | 779 | TGACCAAA GGCTAGCTACAACGA ACTTCCTG | 2044 |
| 427 | AGUGUUUG G UCAAAGGA | 780 | TCCTTTGA GGCTAGCTACAACGA CAAACACT | 2045 |
| 439 | AAGGAAAA A UGAUAAUG | 871 | CATTATCA GGCTAGCTACAACGA TTTTCCTT | 2046 |
| 442 | GAAAAAUG A UAAUGAAU | 872 | ATTCATTA GGCTAGCTACAACGA CATTTTTC | 2047 |
| 445 | AAAUGAUA A UGAAUUCA | 873 | TGAATTCA GGCTAGCTACAACGA TATCATTT | 2048 |
| 449 | GAUAAUGA A UUCAUUGA | 874 | TCAATGAA GGCTAGCTACAACGA TCATTATC | 2049 |
| 453 | AUGAAUUC A UUGAGCCU | 449 | AGGCTCAA GGCTAGCTACAACGA GAATTCAT | 2050 |
| 458 | UUCAUUGA G CCUCUCUG | 781 | CAAAGAGG GGCTAGCTACAACGA TCAATGAA | 2051 |
| 466 | GCCUCUUU C CUGCCAUU | 706 | AATGGCAG GGCTAGCTACAACGA AAAGAGGC | 2052 |
| 469 | UCUUUGCU G CCAUUUCU | 707 | AGAAATGG GGCTAGCTACAACGA AGCAAAGA | 2053 |
| 472 | UUGCUGCC A UUUCUGGA | 455 | TCCAGAAA GGCTAGCTACAACGA GGCAGCAA | 2054 |
| 483 | UUUCUGGA A UGAUUCUU | 875 | AAGAATCA GGCTAGCTACAACGA TCCAGAAA | 2055 |
| 484 | CUGGAAUG A UUCUUUCA | 876 | TGAAAGAA GGCTAGCTACAACGA CATTCCAG | 2056 |
| 493 | UUCUUUCA A UCAUGGAC | 877 | GTCCATGA GGCTAGCTACAACGA TGAAAGAA | 2057 |
| 496 | UUUCAAUC A UGGACAUA | 459 | TATGTCCA GGCTAGCTACAACGA GATTGAAA | 2058 |
| 500 | AAUCAUGG A CAUACUUA | 878 | TAAGTATG GGCTAGCTACAACGA CCATGATT | 2059 |
| 502 | UCAUGGAC A UACUUAAU | 460 | ATTAAGTA GGCTAGCTACAACGA GTCCATGA | 2060 |
| 504 | AUGGACAU A CUUAAUAU | 86 | ATATTAAG GGCTAGCTACAACGA ATGTCCAT | 2061 |
| 509 | CAUACUUA A UAUUAAAA | 879 | TTTTAATA GGCTAGCTACAACGA TAAGTATG | 2062 |
| 511 | UACUUAAU A UUAAAAUU | 89 | AATTTTAA GGCTAGCTACAACGA ATTAAGTA | 2063 |
| 517 | AUAUUAAA A UUUCCCAU | 880 | ATGGGAAA GGCTAGCTACAACGA TTTAATAT | 2064 |
| 524 | AAUUUCCC A UUUUUUAA | 464 | TTAAAAAA GGCTAGCTACAACGA GGGAAATT | 2065 |
| 535 | UUUUAAAA A UGGAGAGU | 881 | ACTCTCCA GGCTAGCTACAACGA TTTTAAAA | 2066 |
| 542 | AAUGGAGA G UCUGAAUU | 782 | AATTCAGA GGCTAGCTACAACGA TCTCCATT | 2067 |
| 548 | GAGUCUGA A UUUUAUUA | 882 | TAATAAAA GGCTAGCTACAACGA TCAGACTC | 2068 |
| 553 | UGAAUUUU A UUAGAGCU | 105 | AGCTCTAA GGCTAGCTACAACGA AAAATTCA | 2069 |
| 559 | UUAGUAGA G CUCACACA | 783 | TGTGTGAG GGCTAGCTACAACGA TCTAATAA | 2070 |
| 563 | UAGAGCUC A CACACCAU | 467 | ATGGTGTG GGCTAGCTACAACGA GAGCTCTA | 2071 |
| 565 | GAGCUCAC A CACCAUAU | 468 | ATATGGTG GGCTAGCTACAACGA GTGAGCTC | 2072 |
| 567 | GCUCACAC A CCAUAUAU | 469 | ATATATGG GGCTAGCTACAACGA GTGTGAGC | 2073 |
| 570 | CACACACC A UAUAUUAA | 471 | TTAATATA GGCTAGCTACAACGA GGTGTGTG | 2074 |
| 572 | CACACCAU A UAUUAACA | 109 | TGTTAATA GGCTAGCTACAACGA ATGGTGTG | 2075 |
| 574 | CACCAUAU A UUAACAUA | 110 | TATGTTAA GGCTAGCTACAACGA ATATGGTG | 2076 |
| 578 | AUAUAUUA A CAUAUACA | 883 | TGTATATG GGCTAGCTACAACGA TAATATAT | 2077 |
| 580 | AUAUUAAC A UAUACAAC | 472 | GTTGTATA GGCTAGCTACAACGA GTTAATAT | 2078 |
| 582 | AUUAACAU A UACAACUG | 113 | CAGTTGTA GGCTAGCTACAACGA ATGTTAAT | 2079 |
| 584 | UAACAUAU A CAACUGUG | 114 | CACAGTTG GGCTAGCTACAACGA ATATGTTA | 2080 |
| 587 | CAUAUACA A CUGUGAAC | 884 | GTTCACAG GGCTAGCTACAACGA TGTATATG | 2081 |
| 590 | AUACAACU G UGAACCAG | 784 | CTGGTTCA GGCTAGCTACAACGA AGTTGTAT | 2082 |
| 594 | AACUGUGA A CCAGCUAA | 885 | TTAGCTGG GGCTAGCTACAACGA TCACAGTT | 2083 |
| 598 | GUGAACCA G CUAAUCCC | 785 | GGGATTAG GGCTAGCTACAACGA TGGTTCAC | 2084 |
| 602 | ACCAGCUA A UCCCUCUG | 886 | CAGAGGGA GGCTAGCTACAACGA TAGCTGGT | 2085 |
| 617 | UGAGAAAA A CUCCCCAU | 887 | ATGGGGAG GGCTAGCTACAACGA TTTTCTCA | 2086 |
| 624 | AACUCCCC A UCUACCCA | 486 | TGGGTAGA GGCTAGCTACAACGA GGGGAGTT | 2087 |
| 628 | CCCCAUCU A CCCAAUAC | 120 | GTATTGGG GGCTAGCTACAACGA AGATGGGG | 2088 |
| 633 | UCUACCCA A UACUGUUA | 888 | TAACAGTA GGCTAGCTACAACGA TGGGTAGA | 2089 |
| 635 | UACCCAAU A CUGUUACA | 121 | TGTAACAG GGCTAGCTACAACGA ATTGGGTA | 2090 |
| 638 | CCAAUACU G UUACAGCA | 786 | TGCTGTAA GGCTAGCTACAACGA AGTATTGG | 2091 |
| 641 | AUACUGUU A CAGCAUAC | 123 | GTATGCTG GGCTAGCTACAACGA AACAGTAT | 2092 |
| 644 | CUGUUACA G CAUACAAU | 787 | ATTGTATG GGCTAGCTACAACGA TGTAACAG | 2093 |
| 646 | GUUACAGC A UACAAUCU | 493 | AGATTGTA GGCTAGCTACAACGA GCTGTAAC | 2094 |
| 648 | UACAGCAU A CAAUCUCU | 124 | AGAGATTG GGCTAGCTACAACGA ATGCTGTA | 2095 |
| 651 | AGCAUACA A UCUCUGUU | 889 | AACAGAGA GGCTACCTACAACGA TGTATGCT | 2096 |
| 657 | CAAUCUCU G UUCUUGGG | 788 | CCCAAGAA GGCTAGCTACAACGA AGAGATTG | 2097 |
| 665 | GUUCUUGG G CAUUUUGU | 789 | ACAAAATG GGCTAGCTACAACGA CCAAGAAC | 2098 |
| 667 | UCUUGGGC A UUUUGUCA | 498 | TGACAAAA GGCTAGCTACAACGA GCCCAAGA | 2099 |
| 672 | GGCAUUUU G UCAGUGAU | 790 | ATCACTGA GGCTAGCTACAACGA AAAATGCC | 2100 |
| 676 | UUUUGUCA G UGAUGCUG | 791 | CAGCATCA GGCTAGCTACAACGA TGACAAAA | 2101 |
| 679 | UGUCAGUG A UGCUGAUC | 890 | GATCAGCA GGCTAGCTACAACGA CACTGACA | 2102 |
| 681 | UCAGUGAU G CUGAUCUU | 713 | AAGATCAG GGCTAGCTACAACGA ATCACTGA | 2103 |
| 685 | UGAUGCUG A UCUUUGCC | 891 | GGCAAAGA GGCTAGCTACAACGA CAGCATCA | 2104 |
| 691 | UGAUCUUU G CCUUCUUC | 715 | GAAGAAGG GGCTAGCTACAACGA AAAGATCA | 2105 |
| 705 | UUCCAGGA A CUUGUAAU | 892 | ATTACAAG GGCTAGCTACAACGA TCCTGGAA | 2106 |
| 709 | AGGAACUU G UAAUAGCU | 792 | AGCTATTA GGCTAGCTACAACGA AAGTTCCT | 2107 |
| 712 | AACUUGUA A UAGCUGGC | 893 | GCCAGCTA GGCTAGCTACAACGA TACAAGTT | 2108 |
| 715 | UUGUAAUA G CUGCCAUC | 793 | GATGCCAG GGCTAGCTACAACGA TATTACAA | 2109 |
| 719 | AAUAGCUG G CAUCGUUG | 794 | CAACGATG GGCTAGCTACAACGA CAGCTATT | 2110 |
| 721 | UAGCUGGC A UCGUUGAG | 509 | CTCAACGA GGCTAGCTACAACGA GCCAGCTA | 2111 |
| 724 | CUGGCAUC G UUGAGAAU | 795 | ATTCTCAA GGCTAGCTACAACGA GATGCCAG | 2112 |
| 731 | CGUUCAGA A UGAAUGGA | 894 | TCCATTCA GGCTAGCTACAACGA TCTCAACG | 2113 |
| 735 | GACAAUGA A UGGAAAAG | 895 | CTTTTCCA GGCTAGCTACAACGA TCATTCTC | 2114 |
| 745 | GGAAAAGA A CGUGCUCC | 896 | GGAGCACG GGCTAGCTACAACGA TCTTTTCC | 2115 |
| 747 | AAAAGAAC G UGCUCCAG | 796 | CTGGAGCA GGCTAGCTACAACGA GTTCTTTT | 2116 |
| 749 | AAGAACGU G CUCCAGAC | 718 | GTCTGGAG GGCTAGCTACAACGA ACGTTCTT | 2117 |
| 756 | UCCUCCAG A CCCAAAUC | 897 | GATTTGGG GGCTAGCTACAACGA CTGGAGCA | 2118 |
| 762 | ACACCCAA A UCUAACAU | 898 | ATGTTAGA GGCTAGCTACAACGA TTGGGTCT | 2119 |
| 767 | CAAAUCUA A CAUAGUUC | 899 | GAACTATG GGCTAGCTACAACGA TAGATTTG | 2120 |
| 769 | AAUCUAAC A UAGUUCUC | 517 | GAGAACTA GGCTAGCTACAACGA GTTAGATT | 2121 |
| 772 | CUAACAUA G UUCUCCUG | 797 | CAGGAGAA GGCTAGCTACAACGA TATGTTAG | 2122 |
| 780 | GUUCUCCU G UCACCAGA | 798 | TCTGCTGA GGCTAGCTACAACGA AGGAGAAC | 2123 |
| 784 | UCCUGUCA G CACAACAA | 799 | TTCTTCTG GGCTACCTACAACGA TGACAGGA | 2124 |
| 801 | AAAAAACA A CAGACUAU | 900 | ATAGTCTG GGCTACCTACAACGA TCTTTTTT | 2125 |
| 805 | AACAACAG A CUAUUGAA | 901 | TTCAATAG GGCTAGCTACAACGA CTGTTCTT | 2126 |
| 808 | AACACACU A UUGAAAUA | 154 | TATTTCAA GGCTAGCTACAACGA AGTCTGTT | 2127 |
| 814 | CUAUUGAA A UAAAAGAA | 902 | TTCTTTTA GGCTAGCTACAACGA TTCAATAG | 2128 |
| 826 | AAGAAGAA G UGGUUGGG | 800 | CCCAACCA GGCTAGCTACAACGA TTCTTCTT | 2129 |
| 829 | AACAAGUC G UUCCGCUA | 801 | TAGCCCAA GGCTACCTACAACGA CACTTCTT | 2130 |
| 834 | CUGGUUGG G CUAACUGA | 802 | TCACTTAC GGCTACCTACAACGA CCAACCAC | 2131 |
| 838 | UUGCGCUA A CUGAAACA | 903 | TGTTTCAG GGCTAGCTACAACGA TAGCCCAA | 2132 |
| 844 | UAACUCAA A CAUCUUCC | 904 | GGAAGATG GGCTAGCTACAACGA TTCAGTTA | 2133 |
| 846 | ACUGAAAC A UCUGCCCA | 527 | TGGGAAGA GGCTAGCTACAACGA CTTTCACT | 2134 |
| 855 | UCUUCCCA A CCAAACAA | 905 | TTCTTTGC GGCTAGCTACAACGA TCGGAACA | 2135 |
| 863 | ACCAAACA A UGAACAAC | 906 | CTTCTTCA GGCTACCTACAACGA TCTTTCCT | 2136 |
| 872 | UCAAGAAC A CAUUCAAA | 907 | TTTCAATG GGCTACCTACAACGA CTTCTTCA | 2137 |
| 874 | AAGAACAC A UUGAAAUU | 534 | AATTTCAA GGCTAGCTACAACGA GTCTTCTT | 2138 |
| 880 | ACAUUCAA A UUAUUCCA | 908 | TGGAATAA GGCTACCTACAACGA TTCAATGT | 2139 |
| 883 | UUGAAAUU A UUCCAAUC | 164 | GATTGGAA GGCTACCTACAACGA AATTTCAA | 2140 |
| 889 | UCAUUCCA A UCCAACAA | 909 | TTCTTGGA GGCTACCTACAACGA TGGAATAA | 2141 |
| 913 | AACAACAA A CACACACG | 910 | CGTCTCTG GGCTACCTACAACGA TTCTTCTT | 2142 |
| 919 | AAACACAC A CCAACUUU | 911 | AAACTTCC GGCTACCTACAACGA CTCTCTTT | 2143 |
| 923 | ACAGACCA A CUUUCCAC | 912 | CTCCAAAG GGCTACCTACAACGA TCGTCTCT | 2144 |
| 933 | UUUCCACA A CCUCCCCA | 913 | TGGGGAGG GGCTAGCTACAACGA TCTGGAAA | 2145 |
| 944 | UCCCCAAC A UCAGCAAU | 914 | ATTCCTCA GGCTACCTACAACGA CTTCGGGA | 2146 |
| 951 | CAUCACCA A UCCUCACC | 915 | CCTCAGCA GGCTACCTACAACGA TCCTGATC | 2147 |
| 957 | CAAUCCUC A CCAAUACA | 552 | TCTATTCC GGCTACCTACAACGA CACCATTC | 2148 |
| 961 | CCUCACCA A UACAAAAU | 916 | ATTTTCTA GGCTACCTACAACGA TCCTCACC | 2149 |
| 968 | AAUACAAA A UCACACCU | 917 | ACCTCTCA GGCTACCTACAACGA TTTCTATT | 2150 |
| 971 | AGAAAAUC A CAGCUCUC | 918 | GACAGCTG GGCTACCTACAACGA CATTTTCT | 2151 |
| 974 | AAAUCACA G CUCUCCUU | 803 | AAGGAGAG GGCTACCTACAACGA TGTCATTT | 2152 |
| 985 | CUCCUUAA G UCAUUUCU | 804 | ACAAATCA GGCTACCTACAACGA TTAACCAC | 2153 |
| 988 | CUUAACUC A UUUCUUCU | 919 | ACAACAAA GGCTACCTACAACGA CACTTAAC | 2154 |
| 997 | UUUCUUCU G UUUUCUGU | 805 | ACACAAAA GGCTACCTACAACGA ACAACAAA | 2155 |
| 1004 | UCUUUUCU G UUUCCUUU | 806 | AAAGGAAA GGCTAGCTACAACGA AGAPAACA | 2156 |
| 1018 | UUUUUUAA A CAUUAGUG | 920 | CACTAATC GGCTAGCTACAACGA TTAAAAAA | 2157 |
| 1020 | UUUUAAAC A UUACUCUU | 565 | AACACTAA GGCTACCTACAACGA CTTTAAAA | 2158 |
| 1024 | AAACAUUA C UCUUCAUA | 807 | TATCAACA GGCTACCTACAACGA TAATCTTT | 2159 |
| 1026 | ACAUUAGU G UUCAUAGC | 808 | GCTATGAA GGCTAGCTACAACGA ACTAATGT | 2160 |
| 1030 | UAGUGUUC A UAGCUUCC | 566 | GGAAGCTA GGCTAGCTACAACGA GAACACTA | 2161 |
| 1033 | UGUUCAUA G CUUCCAAG | 809 | CTTGGAAG GGCTAGCTACAACGA TATGAACA | 2182 |
| 1044 | UCCAAGAG A CAUGCUGA | 921 | TCAGCATG GGCTAGCTACAACGA CTCTTGGA | 2163 |
| 1046 | CAAGAGAC A UGCUGACU | 570 | AGTCAGCA GGCTAGCTACAACGA GTCTCTTG | 2164 |
| 1048 | AGAGACAU G CUGACUUU | 726 | AAAGTCAG GGCTAGCTACAACGA ATGTCTCT | 2165 |
| 1052 | ACAUGCUG A CUUUCAUU | 922 | AATGAAAG GGCTAGCTACAACGA CAGCATGT | 2166 |
| 1058 | UGACUUUC A UUUCUUGA | 573 | TCAAGAAA GGCTAGCTACAACGA GAAAGTCA | 2167 |
| 1068 | UUCUUGAG G UACUCUGC | 810 | GCAGAGTA GGCTAGCTACAACGA CTCAAGAA | 2168 |
| 1070 | CUUGAGGU A CUCUGCAC | 212 | GTGCAGAG GGCTAGCTACAACGA ACCTCAAG | 2169 |
| 1075 | GGUACUCU G CACAUACG | 729 | CGTATGTG GGCTAGCTACAACGA AGAGTACC | 2170 |
| 1077 | UACUCUGC A CAUACGCA | 577 | TGCGTATG GGCTAGCTACAACGA GCAGAGTA | 2171 |
| 1079 | CUCUGCAC A UACGCACC | 578 | GGTGCGTA GGCTAGCTACAACGA GTGCAGAG | 2172 |
| 1081 | CUGCACAU A CGCACCAC | 214 | GTGGTGCG GGCTAGCTACAACGA ATGTGCAG | 2173 |
| 1083 | GCACAUAC G CACCACAU | 730 | ATGTGGTG GGCTAGCTACAACGA GTATGTGC | 2174 |
| 1085 | ACAUACGC A CCACAUCU | 579 | AGATGTGG GGCTAGCTACAACGA GCGTATGT | 2175 |
| 1088 | UACGCACC A CAUCUCUA | 581 | TAGAGATG GGCTAGCTACAACGA GGTGCGTA | 2176 |
| 1090 | CGCACCAC A UCUCUAUC | 582 | GATAGAGA GGCTAGCTACAACGA GTGGTGCG | 2177 |
| 1096 | ACAUCUCU A UCUGGCCU | 217 | AGGCCAGA GGCTAGCTACAACGA AGAGATGT | 2178 |
| 1101 | UCUAUCUG G CCUUUGCA | 811 | TGCAAAGG GGCTAGCTACAACGA CAGATAGA | 2179 |
| 1107 | UGGCCUUU G CAUGGAGU | 731 | ACTCCATG GGCTAGCTACAACGA AAAGGCCA | 2180 |
| 1109 | GCCUUUGC A UGGAGUGA | 588 | TCACTCCA GGCTAGCTACAACGA GCAAAGGC | 2181 |
| 1114 | UCCAUGGA U UGACCAUA | 812 | TATGGTCA GGCTAGCTACAACGA TCCATGCA | 2182 |
| 1117 | AUGGAGUG A CCAUAGCU | 923 | AGCTATGG GGCTAGCTACAACGA CACTCCAT | 2183 |
| 1120 | GAGUGACC A UAGCUCCU | 590 | AGGAGCTA GGCTAGCTACAACGA GGTCACTC | 2184 |
| 1123 | UGACCAUA G CUCCUUCU | 813 | AGAAGGAG GGCTAGCTACAACGA TATGGTCA | 2185 |
| 1137 | UCUCUCUU A CAUUGAAU | 228 | ATTCAATG GGCTAGCTACAACGA AAGAGAGA | 2188 |
| 1139 | UCUCUUAC A UUGAAUGU | 597 | ACATTCAA GGCTAGCTACAACGA GTAAGAGA | 2187 |
| 1144 | UACAUUGA A UGUAGAGA | 924 | TCTCTACA GGCTAGCTACAACGA TCAATGTA | 2188 |
| 1146 | CAUUGAAU G UAGAGAAU | 814 | ATTCTCTA GGCTAGCTACAACGA ATTCAATG | 2189 |
| 1153 | UGUAGAGA A UGUAGCCA | 925 | TGGCTACA GGCTAGCTACAACGA TCTCTACA | 2190 |
| 1155 | UAGAGAAU G UAGCCAUU | 815 | AATGGCTA GGCTAGCTACAACGA ATTCTCTA | 2191 |
| 1158 | AGAAUGUA G CCAUUGUA | 816 | TACAATGG GGCTAGCTACAACGA TACATTCT | 2192 |
| 1181 | AUGUAGCC A UUGUAGCA | 599 | TGCTACAA GGCTAGCTACAACGA GCCTACAT | 2193 |
| 1164 | UAGCCAUU G UAGCAGCU | 817 | AGCTGCTA GGCTAGCTACAACGA AATGGCTA | 2194 |
| 1167 | CCAUUGUA U CAGCUUGU | 818 | ACAAGCTG GGCTAGCTACAACGA TACAATGG | 2195 |
| 1170 | UUGUAGCA G CUUGUGUU | 819 | AACACAAG GGCTAGCTACAACGA TGCTACAA | 2196 |
| 1174 | AGCAGCUU G UGUUGUCA | 820 | TGACAACA GGCTACCTACAACGA AAGCTGCT | 2197 |
| 1176 | CAGCUUGU G UUGUCACG | 821 | CGTGACAA GGCTAGCTACAACGA ACAAGCTG | 2198 |
| 1179 | CUUGUGUU G UCACGCUU | 822 | AAGCGTGA GGCTAGCTACAACGA AACACAAG | 2199 |
| 1182 | GUGUUGUC A CGCUUCUU | 602 | AAGAAGCG GGCTAGCTACAACGA GACAACAC | 2200 |
| 1184 | GUUGUCAC U CUUCUUCU | 734 | AGAAGAAG GGCTAGCTACAACGA GTGACAAC | 2201 |
| 1198 | UCUUUUGA U CAACUUUC | 823 | GAAAGTTG GGCTAGCTACAACGA TCAAAAGA | 2202 |
| 1202 | UUUGAGCA A CUUUCUUA | 926 | TAAGAAAG GGCTAGCTACAACGA TGCTCAAA | 2203 |
| 1209 | ACUUUCUU A CACUGAAG | 248 | CTTCAGTG GGCTAGCTACAACGA AAGAAAGT | 2204 |
| 1211 | UUUCUUAC A CUGAAGAA | 609 | TTCTTCAG GGCTAGCTACAACGA GTAAGAAA | 2205 |
| 1222 | GAAGAAAG U CAGAAUGA | 824 | TCATTCTG GGCTAGCTACAACGA CTTTCTTC | 2206 |
| 1227 | AAGGCAGA A UGAGUGCU | 927 | AGCACTCA GGCTAGCTACAACGA TCTGCCTT | 2207 |
| 1231 | CAGAAUGA U UGCUUCAG | 825 | CTGAAGCA GGCTAGCTACAACGA TCATTCTG | 2208 |
| 1233 | GAAUGAGU U CUUCAGAA | 738 | TTCTGAAG GGCTAGCTACAACGA ACTCATTC | 2209 |
| 1241 | UCUUCAGA A UGUGAUUU | 928 | AAATCACA GGCTAGCTACAACGA TCTGAAGC | 2210 |
| 1243 | UUCAGAAU G UGAUUUCC | 826 | GGAAATCA GGCTAGCTACAACGA ATTCTGAA | 2221 |
| 1246 | AGAAUGUG A UUUCCUAC | 929 | GTAGGAAA GGCTAGCTACAACGA CACATTCT | 2212 |
| 1253 | GAUUUCCU A CUAACCUG | 254 | CAGGTTAG GGCTAGCTACAACGA AGGAAATC | 2213 |
| 1257 | UCCUACUA A CCUGUUCC | 930 | GGAACAGG GGCTAGCTACAACGA TAGTAGGA | 2214 |
| 1261 | ACUAACCU G UUCCUUGG | 827 | CCAAGGAA GGCTAGCTACAACGA AGGTTAGT | 2215 |
| 1270 | UUCCUUGC A UAGGCUUU | 931 | AAAGCCTA GGCTAGCTACAACGA CCAAGGAA | 2216 |
| 1274 | UUGCAUAG G CUUUUUAG | 828 | CTAAAAAG GGCTAGCTACAACGA CTATCCAA | 2217 |
| 1282 | GCUUUUUA G UAUAGUAU | 829 | ATACTATA GGCTAGCTACAACGA TAAAAAGC | 2218 |
| 1284 | UUUUUAGU A UAGUAUUU | 265 | AAATACTA GGCTAGCTACAACGA ACTAAAAA | 2219 |
| 1287 | UUAGUAUA G UAUUUUUU | 830 | AAAAAATA GGCTAGCTACAACGA TATACTAA | 2220 |
| 1289 | AGUAUAGU A UUUUUUUU | 267 | AAAAAAAA GGCTAGCTACAACGA ACTATACT | 2221 |
| 1299 | UUUUUUUU G UCAUUUUC | 831 | GAAAATGA GGCTAGCTACAACGA AAAAAAAA | 2222 |
| 1302 | UUUUUCUC A UUUUCUCC | 622 | GGAGAAAA GGCTAGCTACAACGA GACAAAAA | 2223 |
| 1311 | UUUUCUCC A UCACCAAC | 625 | GTTCCTGA GGCTACCTACAACGA GGAGAAAA | 2224 |
| 1315 | CUCCAUCA G CAACCAGG | 832 | CCTGGTTG GGCTAGCTACAACGA TGATGGAG | 2225 |
| 1318 | CAUCAGCA A CCAGGGAG | 932 | CTCCCTGG GGCTAGCTACAACGA TGCTGATG | 2226 |
| 1327 | CCAGGCAG A CUGCACCU | 933 | AGGTGCAG GGCTAGCTACAACGA CTCCCTGG | 2227 |
| 1330 | GGGAGACU G CACCUGAU | 740 | ATCAGGTG GGCTAGCTACAACGA AGTCTCCC | 2228 |
| 1332 | GAGACUGC A CCUGAUGG | 631 | CCATCAGG GGCTAGCTACAACGA GCAGTCTC | 2229 |
| 1337 | UGCACCUG A UGGAAAAG | 934 | CTTTTCCA GGCTAGCTACAACGA CAGGTCCA | 2230 |
| 1346 | UGGAAAAG A UAUAUGAC | 935 | GTCATATA GGCTAGCTACAACGA CTTTTCCA | 2231 |
| 1348 | GAAAAGAU A UAUGACUG | 283 | CAGTCATA GGCTAGCTACAACGA ATCTTTTC | 2232 |
| 1350 | AAAGAUAU A UGACGGCU | 284 | AGCAGTCA GGCTAGCTACAACGA ATATCTTT | 2233 |
| 1353 | GAUAUAUG A CUGCUUCA | 936 | TGAAGCAG GGCTAGCTACAACGA CATATATC | 2234 |
| 1356 | AUAUGACU G CUUCAUGA | 743 | TCATGAAG GGCTAGCTACAACGA AGTCATAT | 2235 |
| 1361 | ACUGCUUC A UGACAGUC | 636 | GAATGTCA GGCTAGCTACAACGA GAAGCAGT | 2236 |
| 1364 | GCUUCAUG A CAUUCCUA | 937 | TAGGAATG GGCTAGCTACAACGA CATGAAGC | 2237 |
| 1366 | GUCAUGAC A UUCCUAAA | 637 | TTTAGGAA GGCTAGCTACAACGA GTCATGAA | 2238 |
| 1374 | AUUCCUAA A CUAUCUUU | 938 | AAAGATAG GGCTAGCTACAACGA TTAGGAAT | 2239 |
| 1377 | CCUAAACU A UCUUUUUG | 290 | AAAAAAGA GGCTAGCTACAACGA AGTTTAGG | 2240 |
| 1388 | UUUUUUUU A UUCCACAU | 299 | ATGTGGAA GGCTAGCTACAACGA AAAAAAAA | 2241 |
| 1393 | UUUAUUCC A CAUCUACG | 643 | CGTAGATG GGCTAGCTACAACGA GGAATAAA | 2242 |
| 1395 | UAUUCCAC A UCUACGUU | 644 | AACGTAGA GGCTAGCTACAACGA GTGGAATA | 2243 |
| 1399 | CCACAUCU A CGUUUUUG | 303 | CAAAAACG GGCTAGCTACAACGA AGATGTGG | 2244 |
| 1401 | ACAUCUAC G UUUUUGGU | 833 | ACCAAAAA GGCTAGCTACAACGA GTAGATGT | 2245 |
| 1408 | CGUUUUUG G UGGAGUCC | 834 | GGACTCCA GGCTAGCTACAACGA CAAAAACG | 2246 |
| 1413 | TUGGUGGA G UCCCUUUU | 835 | AAAAGGGA GGCTAGCTACAACGA TCCACCAA | 2247 |
| 1422 | UCCCUUUU G CAUCAUUG | 745 | CAATGATG GGCTAGCTACAACGA AAAAGGGA | 2248 |
| 1424 | CCUUUUGC A UCAUUGUU | 649 | AACAATGA GGCTAGCTACAACGA GCAAAAGG | 2249 |
| 1427 | UUUGCAUC A UUGUUUUA | 650 | TAAAACAA GGCTAGCTACAACGA GATGCAAA | 2250 |
| 1430 | GCAUCAUU G UUUUAAGG | 836 | CCTTAAAA GGCTAGCTACAACGA AATGATGC | 2251 |
| 1439 | UUUUAAGG A UGAUAAAA | 939 | TTTTATCA GGCTAGCTACAACGA CCTTAAAA | 2252 |
| 1442 | UAAGGAUG A UAAAAAAA | 940 | TTTTTTTA GGCTAGCTACAACGA CATCCTTA | 2253 |
| 1453 | AAAAAAAA A UAACAACU | 941 | AGTTGTTA GGCTAGCTACAACGA TTTTTTTT | 2254 |
| 1456 | AAAAAAUA A CAACUAGG | 942 | CCTAGTTG GGCTAGCTACAACGA TATTTTTT | 2255 |
| 1459 | AAAUAACA A CUAGGGAC | 943 | GTCCCTAG GGCTAGCTACAACGA TGTTATTT | 2256 |
| 1466 | AACUAGGG A CAAUACAG | 944 | CTGTATTG GGCTAGCTACAACGA CCCTAGTT | 2257 |
| 1469 | UAGGGACA A UACAGAAC | 945 | GTTCTGTA GGCTAGCTACAACGA TGTCCCTA | 2258 |
| 1471 | GGGAGAAU A CAGAACCC | 321 | GGGTTCTG GGCTAGCTACAACGA ATTGTCCC | 2259 |
| 1476 | AAUACAGA A CCCAUUCC | 946 | GGAATGGG GGCTAGCTACAACGA TCTGTATT | 2260 |
| 1480 | CAGAACCC A UUCCAUUU | 657 | AAATGGAA GGCTAGCTACAACGA GGGTTCTG | 2261 |
| 1485 | CCCAUUCC A UUUAUCUU | 659 | AAGATAAA GGCTAGCTACAACGA GGAATGGG | 2262 |
| 1489 | UUCCAUUU A UCUUUCUA | 326 | TAGAAAGA GGCTAGCTACAACGA AAATGGAA | 2263 |
| 1497 | AUCUUUCU A CAGGGCUG | 331 | CACCCCTC GGCTAGCTACAACGA AGAAAGAT | 2264 |
| 1502 | UCUACAGG G CUGACAUU | 837 | AATGTCAG GGCTAGCTACAACGA CCTGTAGA | 2265 |
| 1506 | CAGGGCUG A CAUUGUGG | 947 | CCACAATG GGCTAGCTACAACGA CAGCCCTG | 2266 |
| 1508 | GGGCUGAC A UUGUGGCA | 664 | TGCCACAA GGCTAGCTACAACGA GTCAGCCC | 2267 |
| 1511 | CUGACAUU G UGGCACAU | 838 | ATGTCCCA GGCTAGCTACAACGA AATGTCAG | 2268 |
| 1514 | ACAUUCUG G CACAUUCU | 839 | AGAATGTG GGCTAGCTACAACGA CACAATCT | 2269 |
| 1516 | AUUCUCCC A CAUUCUUA | 665 | TAAGAATG GGCTAGCTACAACGA GCCACAAT | 2270 |
| 1518 | UCUGCCAC A UUCUUAGA | 666 | TCTAAGAA GGCTAGCTACAACGA GTGCCACA | 2271 |
| 1527 | UUCUUAGA G UUACCACA | 840 | TGTGGTAA GGCTAGCTACAACGA TCTAAGAA | 2272 |
| 1530 | UUAGAGUU A CCACACCC | 338 | GGGTGTGG GGCTAGCTACAACGA AACTCTAA | 2273 |
| 1533 | GACCUACC A CACCCCAU | 669 | ATGGGGTG GGCTAGCTACAACGA GGTAACTC | 2274 |
| 1535 | GUUACCAC A CCCCAUGA | 670 | TCATGGGG GGCTAGCTACAACGA GTGGTAAC | 2275 |
| 1540 | CACACCCC A UGAGGGAA | 674 | TTCCCTCA GGCTAGCTACAACGA GGGGTGTG | 2276 |
| 1549 | UGAGGGAA G CUCUAAAU | 841 | ATTTAGAG GGCTAGCTACAACGA TTCCCTCA | 2277 |
| 1556 | AGCUCUAA A UAGCCAAC | 948 | GTTGGCTA GGCTAGCTACAACGA TTAGAGCT | 2278 |
| 1559 | UCUAAAUA G CCAACACC | 842 | GGTGTTGG GGCTAGCTACAACGA TATTTAGA | 2279 |
| 1563 | AAUAGCCA A CACCCAUC | 949 | GATGGGTG GGCTAGCTACAACGA TGGCTATT | 2280 |
| 1565 | UAGCCAAC A CCCAUCUG | 679 | CAGATGCG GGCTAGCTACAACGA GTTGGCTA | 2281 |
| 1569 | CAACACCC A UCUGUUUU | 682 | AAAACAGA GGCTAGCTACAACGA GGGTGTTG | 2282 |
| 1573 | ACCCAUCU G UUUUUUGU | 843 | ACAAAAAA GGCTAGCTACAACGA AGATGGGT | 2283 |
| 1580 | UGUUUUUU G UAAAAACA | 844 | TGTTTTTA GGCTAGCTACAACGA AAAAAACA | 2284 |
| 1586 | UUGUAAAA A CAGCAUAG | 950 | CTATCCTG GGCTAGCTACAACGA TTTTACAA | 2285 |
| 1589 | UAAAAACA G CAUAGCUU | 845 | AAGCTATG GGCTAGCTACAACGA TGTTTTTA | 2286 |

Input Sequence = HSCD20A. Cut Site = R/Y
Stem Length = 8. Core Sequence = GGCTAGCTACAACGA
HSCD20A (Human mRNA for CD20 receptor (S7); 1597 bp)

TABLE VIII
Human CD20 Amberzyme Ribozyme and Substrate Sequence

| | | | | Rz Seq |
|---|---|---|---|---|
| Pos | Substrate | Seq ID | Ribozyme | ID |
| 9 | AACAAACU G CACCCACU | 685 | AGUGGGUG GGAGGAAACUCC CU UCAAGGACAUCGUCCGGG AGUUUGUU | 2287 |
| 18 | CACCCACU G AACUCCGC | 686 | GCGGAGUU GGAGGAAACUCC CU UCAAGGACAUCGUCCGGG AGUGGGUG | 2288 |
| 25 | UGAACUCC G CAGCUAGC | 687 | GCUAGCUG GGAGGAAACUCC CU UCAAGGACAUCGUCCGGG GGAGUUCA | 2289 |
| 28 | ACUCCGCA G CUAGCAUC | 749 | GAUGCUAG GGAGGAAACUCC CU UCAAGGACAUCGUCCGGG UGCGGAGU | 2290 |
| 32 | CGCAGCUA G CAUCCAAA | 750 | UUUGGAUG GGAGGAAACUCC CU UCAAGGACAUCGUCCGGG UAGCUGCG | 2291 |
| 44 | CCAAAUCA G CCCUUGAG | 751 | CUCAAGGG GGAGGAAACUCC CU UCAAGGACAUCGUCCGGG UGAUUUGG | 2292 |
| 50 | CAGCCCUU G AGAUUUGA | 688 | UCAAAUCU GGAGGAAACUCC CU UCAAGGACAUCGUCCGGG AAGGGCUG | 2293 |
| 52 | GCCCUUGA G AUUUGAGG | 951 | CCUCAAAU GGAGGAAACUCC CU UCAAGGACAUCGUCCGGG UCAAGGGC | 2294 |
| 57 | UGAGAUUU G AGGCCUUG | 689 | CAAGGCCU GGAGGAAACUCC CU UCAAGGACAUCGUCCGGG AAAUCUCA | 2295 |
| 59 | AGAUUUGA G GCCUUGGA | 952 | UCCAAGGC GGAGGAAACUCC CU UCAAGGACAUCGUCCGGG UCAAAUCU | 2296 |
| 60 | GAUUUGAG G CCUUGGAG | 752 | CUCCAAGG GGAGGAAACUCC CU UCAAGGACAUCGUCCGGG CUCAAAUC | 2297 |
| 65 | GAGGCCUU G GAGACUCA | 953 | UGAGUCUC GGAGGAAACUCC CU UCAAGGACAUCGUCCGGG AAGGCCUC | 2298 |
| 66 | AGGCCUUG G AGACUCAG | 954 | CUGAGUCU GGAGGAAACUCC CU UCAAGGACAUCGUCCGGG CAAGGCCU | 2299 |
| 68 | GCCUUGGA G ACUCAGGA | 955 | UCCUGAGU GGAGGAAACUCC CU UCAAGGACAUCGUCCGGG UCCAAGGC | 2300 |
| 74 | GAGACUCA G GAGUUUUG | 956 | CAAAACUC GGAGGAAACUCC CU UCAAGGACAUCGUCCGGG UGAGUCUC | 2301 |
| 75 | AGACUCAG G AGUUUUGA | 957 | UCAAAACU GGAGGAAACUCC CU UCAAGGACAUCGUCCGGG CUGAGUCU | 2302 |
| 77 | ACUCAGGA G UUUUGAGA | 753 | UCUCAAAA GGAGGAAACUCC CU UCAAGGACAUCGUCCGGG UCCUGAGU | 2303 |
| 82 | GGAGUUUU G AGAGCAAA | 690 | UUUGCUCU GGAGGAAACUCC CU UCAAGGACAUCGUCCGGG AAAACUCC | 2304 |
| 84 | AGUUUUGA G AGCAAAAU | 958 | AUUUUGCU GGAGGAAACUCC CU UCAAGGACAUCGUCCGGG UCAAAACU | 2305 |
| 86 | UUUUGAGA G CAAAAUGA | 754 | UCAUUUUG GGAGGAAACUCC CU UCAAGGACAUCGUCCGGG UCUCAAAA | 2306 |
| 93 | AGCAAAAU G ACAACACC | 691 | GGUGUUGU GGAGGAAACUCC CU UCAAGGACAUCGUCCGGG AUUUUGCU | 2307 |
| 104 | AACACCCA G AAAUUCAG | 959 | CUGAAUUU GGAGGAAACUCC CU UCAAGGACAUCGUCCGGG UGGGUGUU | 2308 |
| 112 | GAAAUUCA G UAAAUGGG | 755 | CCCAUUUA GGAGGAAACUCC CU UCAAGGACAUCGUCCGGG UGAAUUUC | 2309 |
| 118 | CAGUAAAU G GGACUUUC | 960 | GAAAGUCC GGAGGAAACUCC CU UCAAGGACAUCGUCCGGG AUUUACUG | 2310 |
| 119 | AGUAAAUG G GACUUUCC | 961 | GGAAAGUC GGAGGAAACUCC CU UCAAGGACAUCGUCCGGG CAUUUACU | 2311 |
| 120 | GUAAAUGG G ACUUUCCU | 962 | AGGAAAGU GGAGGAAACUCC CU UCAAGGACAUCGUCCGGG CCAUUUAC | 2312 |
| 129 | ACUUUCCU G GCAGAGCC | 963 | GGCUCUGC GGAGGAAACUCC CU UCAAGGACAUCGUCCGGG AGGAAAGU | 2313 |
| 130 | CUUUCCUG G CAGAGCCA | 756 | UGGCUCUG GGAGGAAACUCC CU UCAAGGACAUCGUCCGGG CAGGAAAG | 2314 |
| 133 | UCCUGGCA G AGCCAAUG | 964 | CAUUGGCU GGAGGAAACUCC CU UCAAGGACAUCGUCCGGG UGCCAGGA | 2315 |
| 135 | CUGGCAGA G CCAAUGAA | 757 | UUCAUUGG GGAGGAAACUCC CU UCAAGGACAUCGUCCGGG UCUGCCAG | 2316 |
| 141 | GAGCCAAU G AAAGGCCC | 692 | GGGCCUUU GGAGGAAACUCC CU UCAAGGACAUCGUCCGGG AUUGGCUC | 2317 |
| 145 | CAAUGAAA G GCCCUAUU | 965 | AAUAGGGC GGAGGAAACUCC CU UCAAGGACAUCGUCCGGG UUUCAUUG | 2318 |
| 146 | AAUGAAAG G CCCUAUUG | 758 | CAAUAGGG GGAGGAAACUCC CU UCAAGGACAUCGUCCGGG CUUUCAUU | 2319 |
| 154 | GCCCUAUU G CUAUGCAA | 693 | UUGCAUAG GGAGGAAACUCC CU UCAAGGACAUCGUCCGGG AAUAGGGC | 2320 |
| 159 | AUUGCUAU G CAAUCUGG | 694 | CCAGAUUG GGAGGAAACUCC CU UCAAGGACAUCGUCCGGG AUAGCAAU | 2321 |
| 166 | UGCAAUCU G GUCCAAAA | 966 | UUUUGGAC GGAGGAAACUCC CU UCAAGGACAUCGUCCGGG AGAUUGCA | 2322 |
| 167 | GCAAUCUG G UCCAAAAC | 759 | GUUUUGGA GGAGGAAACUCC CU UCAAGGACAUCGUCCGGG CAGAUUGC | 2323 |
| 185 | ACUCUUCA G GAGGAUGU | 967 | ACAUCCUC GGAGGAAACUCC CU UCAAGGACAUCGUCCGGG UGAAGAGU | 2324 |
| 186 | CUCUUCAG G AGGAUGUC | 968 | GACAUCCU GGAGGAAACUCC CU UCAAGGACAUCGUCCGGG CUGAAGAG | 2325 |
| 188 | CUUCAGGA G GAUGUCUU | 96 | AAGACAUC GGAGGAAACUCC CU UCAAGGACAUCGUCCGGG UCCUGAAG | 2326 |
| 189 | UUCAGGAG G AUGUCUUC | 970 | GAAGACAU GGAGGAAACUCC CU UCAAGGACAUCGUCCGGG CUCCUGAA | 2327 |
| 192 | AGGAGGAU G UCUUCACU | 760 | AGUGAAGA GGAGGAAACUCC CU UCAAGGACAUCGUCCGGG AUCCUCCU | 2328 |
| 201 | UCUUCACU G GUGGGCCC | 971 | GGGCCCAC GGAGGAAACUCC CU UCAAGGACAUCGUCCGGG AGUGAAGA | 2329 |
| 202 | CUUCACUG G UGGGCCCC | 761 | GGGGCCCA GGAGGAAACUCC CU UCAAGGACAUCGUCCGGG CAGUGAAG | 2330 |
| 204 | UCACUGGU G GGCCCCAC | 972 | GUGGGGCC GGAGGAAACUCC CU UCAAGGACAUCGUCCGGG ACCAGUGA | 2331 |
| 205 | CACUGGUG G GCCCCACG | 973 | CGUGGGGC GGAGGAAACUCC CU UCAAGGACAUCGUCCGGG CACCAGUG | 2332 |
| 206 | ACUGGUGG G CCCCACGC | 762 | GCGUGGGG GGAGGAAACUCC CU UCAAGGACAUCGUCCGGG CCACCAGU | 2333 |
| 213 | GGCCCCAC G CAAAGCUU | 695 | AAGCUUUG GGAGGAAACUCC CU UCAAGGACAUCGUCCGGG GUGGGGCC | 2334 |
| 218 | CACGCAAA G CUUCUUCA | 763 | UGAAGAAG GGAGGAAACUCC CU UCAAGGACAUCGUCCGGG UUUGCGUG | 2335 |
| 228 | UUCUUCAU G AGGGAAUC | 696 | CAUUCCCU GGAGGAAACUCC CU UCAAGGACAUCGUCCGGG AUGAAGAA | 2336 |
| 230 | CUUCAUGA G GGAAUCUA | 974 | UAGAUUCC GGAGGAAACUCC CU UCAAGGACAUCGUCCGGG UCAUGAAG | 2337 |
| 231 | UUCAUGAG G GAAUCUAA | 975 | UUAGAUUC GGAGGAAACUCC CU UCAAGGACAUCGUCCGGG CUCAUGAA | 2338 |
| 232 | UCAUGAGG G AAUCUAAG | 976 | CUUAGAUU GGAGGAAACUCC CU UCAAGGACAUCGUCCGGG CCUCAUGA | 2339 |
| 240 | GAAUCUAA G ACUUUGGG | 977 | CCCAAAGU GGAGGAAACUCC CU UCAAGGACAUCGUCCGGG UUAGAUUC | 2340 |
| 246 | AAGACUUU G GGGGCUGU | 978 | ACAGCCCC GGAGGAAACUCC CU UCAAGGACAUCGUCCGGG AAAGUCUU | 2341 |
| 247 | AGACUUUG G GGGCUGUC | 979 | GACAGCCC GGAGGAAACUCC CU UCAAGGACAUCGUCCGGG CAAAGUCU | 2342 |
| 248 | GACUUUGG G GGCUGUCC | 980 | GGACAGCC GGAGGAAACUCC CU UCAAGGACAUCGUCCGGG CCAAAGUC | 2343 |
| 249 | ACUUUGGG G GCUGUCCA | 981 | UGGACAGC GGAGGAAACUCC CU UCAAGGACAUCGUCCGGG CCCAAAGU | 2344 |
| 250 | CUUUGGGG G CUGUCCAG | 764 | CUGGACAG GGAGGAAACUCC CU UCAAGGACAUCGUCCGGG CCCCAAAG | 2345 |
| 253 | UGGGGGCU G UCCAGAUU | 765 | AAUCUGGA GGAGGAAACUCC CU UCAAGGACAUCGUCCGGG AGCCCCCA | 2346 |
| 258 | GCUGUCCA G AUUAUGAA | 982 | UUCAUAAU GGAGGAAACUCC CU UCAAGGACAUCGUCCGGG UGGACAGC | 2347 |
| 264 | CAGAUUAU G AAUGGGCU | 697 | AGCCCAUU GGAGGAAACUCC CU UCAAGGACAUCGUCCGGG AUAAUCUG | 2348 |
| 268 | UUAUGAAU G GGCUCUUC | 983 | GAAGAGCC GGAGGAAACUCC CU UCAAGGACAUCGUCCGGG AUUCAUAA | 2349 |
| 269 | UAUGAAUG G GCUCUUCC | 984 | GGAAGAGC GGAGGAAACUCC CU UCAAGGACAUCGUCCGGG CAUUCAUA | 2350 |
| 270 | AUGAAUGG G CUCUUCCA | 766 | UGGAAGAG GGAGGAAACUCC CU UCAAGGACAUCGUCCGGG CCAUUCAU | 2351 |
| 283 | UCCACAUU G CCCUGGGG | 698 | CCCCAGGG GGAGGAAACUCC CU UCAAGGACAUCGUCCGGG AAUGUGGA | 2352 |
| 288 | AUUGCCCU G GGGGGUCU | 985 | AGACCCCC GGAGGAAACUCC CU UCAAGGACAUCGUCCGGG AGGGCAAU | 2353 |
| 289 | UUGCCCUG G GGGGUCUU | 986 | AAGACCCC GGAGGAAACUCC CU UCAAGGACAUCGUCCGGG CAGGGCAA | 2354 |
| 290 | UGCCCUGG G GGGUCUUC | 987 | GAAGACCC GGAGGAAACUCC CU UCAAGGACAUCGUCCGGG CCAGGGCA | 2355 |
| 291 | GCCCUGGG G GGUCUUCU | 988 | AGAAGACC GGAGGAAACUCC CU UCAAGGACAUCGUCCGGG CCCAGGGC | 2356 |
| 292 | CCCUGGGG G GUCUUCUG | 989 | CAGAAGAC GGAGGAAACUCC CU UCAAGGACAUCGUCCGGG CCCCAGGG | 2357 |
| 293 | CCUGGGGG G UCUUCUGA | 767 | UCAGAACA GGAGGAAACUCC CU UCAAGGACAUCGUCCGGG CCCCCAGG | 2358 |
| 300 | GGUCUUCU G AUGAUCCC | 699 | GGGAUCAU GGAGGAAACUCC CU UCAAGGACAUCGUCCGGG AGAAGACC | 2359 |
| 303 | CUUCUGAU G AUCCCAGC | 700 | GCUGGGAU GGAGGAAACUCC CU UCAAGGACAUCGUCCGGG AUCAGAAG | 2360 |
| 310 | UGAUCCCA G CAGGCAUC | 768 | GAUCCCUG GGAGGAAACUCC CU UCAAGGACAUCGUCCGGG UGGGAUCA | 2361 |
| 313 | UCCCAGCA G GGAUCUAU | 990 | AUAGAUCC GGAGGAAACUCC CU UCAAGGACAUCGUCCGGG UGCUGGGA | 2362 |
| 314 | CCCAGCAG G GAUCUAUG | 991 | CAUAGAUC GGAGGAAACUCC CU UCAAGGACAUCGUCCGGG CUGCUGGG | 2363 |
| 315 | CCAGCAGG G AUCUAUGC | 992 | GCAUAGAU GGAGGAAACUCC CU UCAAGGACAUCGUCCGGG CCUGCUGG | 2364 |
| 322 | GGAUCUAU G CACCCAUC | 701 | GAUGGGUG GGAGGAAACUCC CU UCAAGGACAUCGUCCGGG AUAGAUCC | 2365 |
| 332 | ACCCAUCU G UGUGACUG | 769 | CAGUCACA GGAGGAAACUCC CU UCAAGGACAUCGUCCGGG AGAUGGGU | 2366 |
| 334 | CCAUCUGU G UGACUGUG | 770 | CACAGUCA GGAGGAAACUCC CU UCAAGGACAUCGUCCGGG ACAGAUGG | 2367 |
| 336 | AUCUGUGU G ACUGUGUG | 702 | CACACAGU GGAGGAAACUCC CU UCAAGGACAUCGUCCGGG ACACAGAU | 2368 |
| 340 | GUGUGACU G UGUGGUAC | 771 | GUACCACA GGAGGAAACUCC CU UCAAGGACAUCGUCCGGG AGUCACAC | 2369 |
| 342 | GUGACUGU G UGGUACCC | 772 | GGGUACCA GGAGGAAACUCC CU UCAAGGACAUCGUCCGGG ACAGUCAC | 2370 |
| 344 | GACUGUGU G GUACCCUC | 993 | CAGGGUAC GGAGGAAACUCC CU UCAAGGACAUCGUCCGGG ACACAGUC | 2371 |
| 345 | ACUGUGUG G UACCCUCU | 773 | AGAGGGUA GGAGGAAACUCC CU UCAAGGACAUCGUCCGGG CACACAGU | 2372 |
| 356 | CCCUCUCU G GGGAGGCA | 994 | UGCCUCCC GGAGGAAACUCC CU UCAAGGACAUCGUCCGGG AGAGAGGG | 2373 |
| 357 | CCUCUCUG G GGAGGCAU | 995 | AUGCCUCC GGAGGAAACUCC CU UCAAGGACAUCGUCCGGG CAGAGAGG | 2374 |
| 358 | CUCUCUGG G GAGGCAUU | 996 | AAUGCCUC GGAGGAAACUCC CU UCAAGGACAUCGUCCGGG CCAGAGAG | 2375 |
| 359 | UCUCUGGG G AGGCAUUA | 997 | UAAUGCCU GGAGGAAACUCC CU UCAAGGACAUCGUCCGGG CCCAGAGA | 2376 |
| 361 | UCUGGGGA G GCAUUAUG | 998 | CAUAAUGC GGAGGAAACUCC CU UCAAGGACAUCGUCCGGG UCCCCAGA | 2377 |
| 362 | CUGGGGAG G CAUUAUGU | 774 | ACAUAAUG GGAGGAAACUCC CU UCAAGGACAUCGUCCGGG CUCCCCAG | 2378 |
| 369 | GGCAUUAU G UAUAUUAU | 775 | AUAAUAUA GGAGGAAACUCC CU UCAAGGACAUCGUCCGGC AUAAUGCC | 2379 |
| 382 | UUAUUUCC G GAUCACUC | 999 | GAGUGAUC GGAGGAAACUCC CU UCAAGGACAUCGUCCGGG GGAAAUAA | 2380 |
| 383 | UAUUUCCG G AUCACUCC | 1000 | GGAGUGAU GGAGGAAACUCC CU UCAAGGACAUCGUCCGGG CGGAAAUA | 2381 |
| 393 | UCACUCCU G GCAGCAAC | 1001 | GUUGCUGC GGAGGAAACUCC CU UCAAGGACAUCGUCCGGG AGGAGUGA | 2382 |
| 394 | CACUCCUG G CAGCAACG | 776 | CGUUGCUG GGAGGAAACUCC CU UCAAGGACAUCGUCCGGG CAGGAGUG | 2383 |
| 397 | UCCUGGCA G CAACGGAG | 777 | CUCCGUUG GGAGGAAACUCC CU UCAAGGACAUCGUCCCGG UGCCAGGA | 2384 |
| 402 | GCAGCAAC G GAGAAAAA | 1002 | UUUUUCUC GGAGGAAACUCC CU UCAAGGACAUCGUCCGGG GUUGCUGC | 2385 |
| 403 | CAGCAACG G AGAAAAAC | 1003 | GUUUUUCU GGAGGAAACUCC CU UCAAGGACAUCGUCCGGG CGUUGCUG | 2386 |
| 405 | GCAACGGA G AAAAACUC | 1004 | GAGUUUUU GGAGGAAACUCC CU UCAAGGACAUCGUCCGGG UCCGUUGC | 2387 |
| 416 | AAACUCCA G GAAGUGUU | 1005 | AACACUUC GGAGGAAACUCC CU UCAAGGACAUCGUCCGGG UGGAGUUU | 2388 |
| 417 | AACUCCAG G AAGUGUUU | 1006 | AAACACUU GGAGGAAACUCC CU UCAAGGACAUCGUCCGGG CUGGAGUU | 2389 |
| 420 | UCCAGGAA G UGUUUGGU | 778 | ACCAAACA GGAGGAAACUCC CU UCAAGGACAUCGUCCGCG UUCCUGGA | 2390 |
| 422 | CAGGAAGU G UUUGGUCA | 779 | UGACCAAA GGAGGAAACUCC CU UCAAGGACAUCGUCCGGG ACUUCCUG | 2391 |
| 426 | AAGUGUUU G GUCAAAGG | 1007 | CCUUUGAC GGAGGAAACUCC CU UCAAGGACAUCGUCCGGG AAACACUU | 2392 |
| 427 | AGUGUUUG G UCAAAGGA | 780 | UCCUUUGA GGAGGAAACUCC CU UCAAGGACAUCGUCCGGG CAAACACU | 2393 |
| 433 | UGGUCAAA G GAAAAAUG | 1008 | CAUUUUUC GGAGGAAACUCC CU UCAAGGACAUCGUCCGGG UUUGACCA | 2394 |
| 434 | GGUCAAAG G AAAAAUGA | 1009 | UCAUUUUU GGAGGAAACUCC CU UCAAGGACAUCGUCCGGG CUUUGACC | 2395 |
| 441 | GGAAAAAU G AUAAUGAA | 703 | UUCAUUAU GGAGGAAACUCC CU UCAAGGACAUCGUCCGGG AUUUUUCC | 2396 |
| 447 | AUGAUAAU G AAUUCAUU | 704 | AAUGAAUU GGAGGAAACUCC CU UCAAGGACAUCGUCCGGG AUUAUCAU | 2397 |
| 456 | AAUUCAUU G AGCCUCUU | 705 | AAGAGGCU GGAGGAAACUCC CU UCAAGGACAUCGUCCGGG AAUGAAUU | 2398 |
| 458 | UUCAUUGA G CCUCUUUG | 781 | CAAAGAGG GGAGGAAACUCC CU UCAAGGACAUCGUCCGGG UCAAUGAA | 2399 |
| 466 | GCCUCUUU G CUGCCAUU | 706 | AAUGGCAG GGAGGAAACUCC CU UCAAGGACAUCGUCCGGG AAAGAGGC | 2400 |
| 469 | UCUUUGCU G CCAUUUCU | 707 | AGAAAUGG GGAGGAAACUCC CU UCAAGGACAUCGUCCGGG AGCAAAGA | 2401 |
| 478 | CCAUUUCU G GAAUGAUU | 1010 | AAUCAUUC GGAGGAAACUCC CU UCAAGGACAUCGUCCGGG AGAAAUGG | 2402 |
| 479 | CAUUUCUG G AAUGAUUC | 1011 | GAAUCAUU GGAGGAAACUCC CU UCAAGGACAUCGUCCGGG CAGAAAUG | 2403 |
| 483 | UCUGGAAU G AUUCUUUC | 708 | GAAAGAAU GGAGGAAACUCC CU UCAAGGACAUCGUCCGGG AUUCCAGA | 2404 |
| 498 | UCAAUCAU G GACAUACU | 1012 | AGUAUGUC GGAGGAAACUCC CU UCAAGGACAUCGUCCGGG AUGAUUGA | 2405 |
| 499 | CAAUCAUG G ACAUACUU | 1013 | AAGUAUGU GGAGGAAACUCC CU UCAAGGACAUCGUCCGGC CAUGAUUG | 2406 |
| 537 | UUAAAAAU G GAGAGUCU | 1014 | AGACUCUC GGAGGAAACUCC CU UCAAGGACAUCGUCCGGG AUUUUUAA | 2407 |
| 538 | UAAAAAUG G AGAGUCUG | 1015 | CAGACUCU GGAGGAAACUCC CU UCAAGGACAUCGUCCGGG CAUUUUUA | 2408 |
| 540 | AAAAUGGA G AGUCUGAA | 1016 | UUCAGACU GGAGGAAACUCC CU UCAAGGACAUCGUCCGGG UCCAUUUU | 2409 |
| 542 | AAUGGAGA G UCUGAAUU | 782 | AAUUCAGA GGAGGAAACUCC CU UCAAGGACAUCGUCCGGG UCUCCAUU | 2410 |
| 546 | GAGAGUCU G AAUUUUAU | 709 | AUAAAAUU GGAGGAAACUCC CU UCAAGGACAUCGUCCGGG AGACUCUC | 2411 |
| 557 | UUUUAUUA G AGCUCACA | 1017 | UGUGAGCU GGAGGAAACUCC CU UCAAGGACAUCGUCCGGG UAAUAAAA | 2412 |
| 559 | UUAUUAGA G CUCACACA | 783 | UGUGUGAG GGAGGAAACUCC CU UCAAGGACAUCGUCCGGG UCUAAUAA | 2413 |
| 590 | AUACAACU G UGAACCAG | 784 | CUGGUUCA GGAGGAAACUCC CU UCAAGGACAUCGUCCGGG AGUUGUAU | 2414 |
| 592 | ACAACUGU G AACCAGCU | 710 | AGCUGGUU GGAGGAAACUCC CU UCAAGGACAUCGUCCGGG ACAGUUGU | 2415 |
| 598 | GUGAACCA G CUAAUCCC | 785 | GGGAUUAG GGAGGAAACUCC CU UCAAGGACAUCGUCCGGG UGGUUCAC | 2416 |
| 610 | AUCCCUCU G AGAAAAAC | 711 | GUUUUUCU GGAGGAAACUCC CU UCAAGGACAUCGUCCGGG ACAGGGAU | 2417 |
| 612 | CCCUCUGA G AAAAACUC | 1018 | GAGUUUUU GGAGGAAACUCC CU UCAAGGACAUCGUCCGGG UCAGAGGG | 2418 |
| 638 | CCAAUACU G UUACAGCA | 786 | UGCUGUAA GGAGGAAACUCC CU UCAAGGACAUCGUCCGGG AGUAUUGG | 2419 |
| 644 | CUGUUACA G CAUACAAU | 787 | AUUGUAUG GGAGGAAACUCC CU UCAAGGACAUCGUCCCGG UGUAACAG | 2420 |
| 657 | CAAUCUCU G UUCUUGGG | 788 | CCCAAGAA GGAGGAAACUCC CU UCAAGGACAUCGUCCGGG AGAGAUUG | 2421 |
| 663 | CUGUUCUU G GGCAUUUU | 1019 | AAAAUGCC GGAGGAAACUCC CU UCAAGGACAUCGUCCGGG AAGAACAG | 2422 |
| 664 | UGUUCUUG G GCAUUUUG | 1020 | CAAAAUGC GGAGGAAACUCC CU UCAAGGACAUCGUCCGGG CAAGAACA | 2423 |
| 665 | GUUCUUGG G CAUUUUGU | 789 | ACAAAAUG GGAGGAAACUCC CU UCAAGGACAUCGUCCGGG CCAAGAAC | 2424 |
| 672 | GGCAUUUU G UCAGUGAU | 790 | AUCACUGA GGAGGAAACUCC CU UCAAGGACAUCGUCCGGG AAAAUGCC | 2425 |
| 676 | UUUUGUCA G UGAUGCUG | 791 | CAGCAUCA GGAGGAAACUCC CU UCAAGGACAUCGUCCGGG UGACAAAA | 2426 |
| 678 | UUGUCAGU G AUGCUGAU | 712 | AUCAGCAU GGAGGAAACUCC CU UCAAGGACAUCGUCCGGG ACUGACAA | 2427 |
| 681 | UCAGUGAU G CUGAUCUU | 713 | AAGAUCAG GGAGGAAACUCC CU UCAAGGACAUCGUCCGGG AUCACUGA | 2428 |
| 684 | GUGAUGCU G AUCUUUGC | 714 | GCAAAGAU GGAGGAAACUCC CU UCAAGGACAUCGUCCGGG AGCAUCAC | 2429 |
| 691 | UGAUCUUU G CCUUCUUC | 715 | GAAGAAGG GGAGGAAACUCC CU UCAAGGACAUCGUCCGGG AAAGAUCA | 2430 |
| 702 | UUCUUCCA G GAACUUGU | 1021 | ACAAGUUC GGAGGAAACUCC CU UCAAGGACAUCGUCCGGG UGGAAGAA | 2431 |
| 703 | UCUUCCAG G AACUUGUA | 1022 | UACAAGUU GGAGGAAACUCC CU UCAAGGACAUCGUCCGGG CUGGAAGA | 2432 |
| 709 | AGGAACUU G UAAUAGCU | 792 | AGCUAUUA GGAGGAAACUCC CU UCAAGGACAUCGUCCGGG AAGUUCCU | 2433 |
| 715 | UUGUAAUA G CUGGCAUC | 793 | GAUGCCAG GGAGGAAACUCC CU UCAAGGACAUCGUCCGGG UAUUACAA | 2434 |
| 718 | UAAUAGCU G GCAUCGUU | 1023 | AACGAUGC GGAGGAAACUCC CU UCAAGGACAUCGUCCGGG AGCUAUUA | 2435 |
| 719 | AAUAGCUG G CAUCGUUG | 794 | CAACGAUG GGAGGAAACUCC CU UCAAGGACAUCGUCCGGG CAGCUAUU | 2436 |
| 724 | CUGGCAUC G UUGAGAAU | 795 | AUUCUCAA GGAGGAAACUCC CU UCAAGGACAUCGUCCGGG GAUGCCAG | 2437 |
| 727 | GCAUCGUU G AGAAUGAA | 716 | UUCAUUCU GGAGGAAACUCC CU UCAAGGACAUCGUCCGGG AACGAUGC | 2438 |
| 729 | AUCGUUGA G AAUGAAUG | 1024 | CAUUCAUU GGAGGAAACUCC CU UCAAGGACAUCGUCCGGG UCAACGAU | 2439 |
| 733 | UUGAGAAU G AAUGGAAA | 717 | UUUCCAUU GGAGGAAACUCC CU UCAAGGACAUCGUCCGGG AUUCUCAA | 2440 |
| 737 | GAAUGAAU G GAAAAGAA | 1025 | UUCUUUUC GGAGGAAACUCC CU UCAAGGACAUCGUCCGGG AUUCAUUC | 2441 |
| 738 | AAUGAAUG G AAAAGAAC | 1026 | GUUCUUUU GGAGGAAACUCC CU UCAAGGACAUCGUCCGGG CAUUCAUU | 2442 |
| 743 | AUGGAAAA G AACGUGCU | 1027 | AGCACGUU GGAGGAAACUCC CU UCAAGGACAUCGUCCGGG UUUUCCAU | 2443 |
| 747 | AAAAGAAC G UGCUCCAG | 796 | CUGGAGCA GGAGGAAACUCC CU UCAAGGACAUCGUCCGGG GUUCUUUU | 2444 |
| 749 | AAGAACGU G CUCCAGAC | 718 | GUCUGGAG GGAGGAAACUCC CU UCAAGGACAUCGUCCGGG ACGUUCUU | 2445 |
| 755 | GUGCUCCA G ACCCAAAU | 1028 | AUUUGGGU GGAGGAAACUCC CU UCAAGGACAUCGUCCGGG UGGAGCAC | 2446 |
| 772 | CUAACAUA G UUCUCCUG | 797 | CAGGAGAA GGAGGAAACUCC CU UCAAGGACAUCGUCCGGG UAUGUUAG | 2447 |
| 780 | GUUCUCCU G UCAGCAGA | 798 | UCUGCUGA GGAGGAAACUCC CU UCAAGGACAUCGUCCGGG AGGAGAAC | 2448 |
| 784 | UCCUGUCA G CAGAAGAA | 799 | UUCUUCUG GGAGGAAACUCC CU UCAAGGACAUCGUCCGGG UGACAGGA | 2449 |
| 787 | UGUCAGCA G AAGAAAAA | 1029 | UUUUUCUU GGAGGAAACUCC CU UCAAGGACAUCGUCCGGG UGCUGACA | 2450 |
| 790 | CAGCAGAA G AAAAAAAA | 1030 | UUUUUUUU GGAGGAAACUCC CU UCAAGGACAUCGUCCGGG UUCUGCUG | 2451 |
| 799 | AAAAAAAA G AACAGACU | 1031 | AGUCUGUU GGAGGAAACUCC CU UCAAGGACAUCGUCCGGG UUUUUUUU | 2452 |
| 804 | AAAGAACA G ACUAUUGA | 1032 | UCAAUAGU GGAGGAAACUCC CU UCAAGGACAUCGUCCGGG UGUUCUUU | 2453 |
| 811 | AGACUAUU G AAAUAAAA | 719 | UUUUAUUU GGAGGAAACUCC CU UCAAGGACAUCGUCCGGG AAUAGUCU | 2454 |
| 820 | AAAUAAAA G AAGAAGUG | 1033 | CACUUCUU GGAGGAAACUCC CU UCAAGGACAUCGUCCGGG UUUUAUUU | 2455 |
| 823 | UAAAAGAA G AAGUGGUU | 1034 | AACCACUU GGAGGAAACUCC CU UCAAGGACAUCGUCCGGG UUCUUUUA | 2456 |
| 826 | AAGAAGAA G UGGUUGGG | 800 | CCCAACCA GGAGGAAACUCC CU UCAAGGACAUCGUCCGGG UUCUUCUU | 2457 |
| 828 | GAAGAAGU G GUUGGGCU | 1035 | AGCCCAAC GGAGGAAACUCC CU UCAAGGACAUCGUCCGGG ACUUCUUC | 2458 |
| 829 | AAGAAGUG G UUGGGCUA | 801 | UAGCCCAA GGAGGAAACUCC CU UCAAGGACAUCGUCCGGG CACUUCUU | 2459 |
| 832 | AAGUGGUU G GGCUAACU | 1036 | AGUUAGCC GGAGGAAACUCC CU UCAAGGACAUCGUCCGGG AACCACUU | 2460 |
| 833 | AGUGGUUG G GCUAACUG | 1037 | CAGUUAGC GGAGGAAACUCC CU UCAAGGACAUCGUCCGGG CAACCACU | 2461 |
| 834 | GUGGUUGG G CUAACUGA | 802 | UCAGUUAG GGAGGAAACUCC CU UCAAGGACAUCGUCCGGG CCAACCAC | 2462 |
| 841 | GGCUAACU G AAACAUCU | 720 | AGAUGUUU GGAGGAAACUCC CU UCAAGGACAUCGUCCGGG AGUUAGCC | 2463 |
| 861 | CAACCAAA G AAUGAAGA | 1038 | UCUUCAUU GGAGGAAACUCC CU UCAAGGACAUCGUCCGGG UUUGGUUG | 2464 |
| 865 | CAAAGAAU G AAGAAGAC | 721 | GUCUUCUU GGAGGAAACUCC CU UCAAGGACAUCGUCCGGG AUUCUUUG | 2465 |
| 868 | AGAAUGAA G AAGACAUU | 1039 | AAUGUCUU GGAGGAAACUCC CU UCAAGGACAUCGUCCGGG UUCAUUCU | 2466 |
| 871 | AUGAAGAA G ACAUUGAA | 1040 | UUCAAUGU GGAGGAAACUCC CU UCAAGGACAUCGUCCGGG UUCUUCAU | 2467 |
| 877 | AAGACAUU G AAAUUAUU | 722 | AAUAAUUU GGAGGAAACUCC CU UCAAGGACAUCGUCCGGG AAUGUCUU | 2468 |
| 895 | CAAUCCAA G AAGAGGAA | 1041 | UUCCUCUU GGAGGAAACUCC CU UCAAGGACAUCGUCCGGG UUGGAUUG | 2469 |
| 898 | UCCAAGAA G AGGAAGAA | 1042 | UUCUUCCU GGAGGAAACUCC CU UCAAGGACAUCGUCCGGG UUCUUGGA | 2470 |
| 900 | CAAGAAGA G GAAGAAGA | 1043 | UCUUCUUC GGAGGAAACUCC CU UCAAGGACAUCGUCCGGG UCUUCUUG | 2471 |
| 901 | AAGAAGAG G AAGAAGAA | 1044 | UUCUUCUU GGAGGAAACUCC CU UCAAGGACAUCGUCCGGG CUCUUCUU | 2472 |
| 904 | AAGAGGAA G AAGAAGAA | 1045 | UUCUUCUU GGAGGAAACUCC CU UCAAGGACAUCGUCCGGG UUCCUCUU | 2473 |
| 907 | AGGAAGAA G AAGAAACA | 1046 | UGUUUCUU GGAGGAAACUCC CU UCAAGGACAUCGUCCGGG UUCUUCCU | 2474 |
| 910 | AAGAAGAA G AAACAGAG | 1047 | CUCUGUUU GGAGGAAACUCC CU UCAAGGACAUCGUCCGGG UUCUUCUU | 2475 |
| 916 | AAGAAACA G AGACGAAC | 1048 | GUUCGUCU GGAGGAAACUCC CU UCAAGGACAUCGUCCGGG UGUUUCUU | 2476 |
| 918 | GAAACAGA G ACGAACUU | 1049 | AAGUUCGU GGAGGAAACUCC CU UCAAGGACAUCGUCCGGC UCUGUUUC | 2477 |
| 921 | ACAGAGAC G AACUUUCC | 723 | GGAAAGUU GGAGGAAACUCC CU UCAAGGACAUCGUCCGGG GUCUCUGU | 2478 |
| 931 | ACUUUCCA G AACCUCCC | 1050 | GGGAGGUU GGAGGAAACUCC CU UCAAGGACAUCGUCCGGG UGGAAAGU | 2479 |
| 943 | CUCCCCAA G AUCAGGAA | 1051 | UUCCUGAU GGAGGAAACUCC CU UCAAGGACAUCGUCCGGG UUGGGGAG | 2480 |
| 948 | CAAGAUCA G CAAUCCUC | 1052 | GAGGAUUC GGAGGAAACUCC CU UCAAGGACAUCGUCCGGG UGAUCUUG | 2481 |
| 949 | AAGAUCAG G AAUCCUCA | 1053 | UGAGGAUU GGAGGAAACUCC CU UCAAGGACAUCGUCCGGG CUGAUCUU | 2482 |
| 964 | CACCAAUA G AAAAUGAC | 1054 | GUCAUUUU GGAGGAAACUCC CU UCAAGGACAUCGUCCGGG UAUUGGUG | 2483 |
| 970 | UAGAAAAU G ACAGCUCU | 724 | AGAGCUGU GGAGGAAACUCC CU UCAAGGACAUCGUCCGGG AUUUUCUA | 2484 |
| 974 | AAAUGACA G CUCUCCUU | 803 | AAGGAGAG GGAGGAAACUCC CU UCAAGGACAUCGUCCGGG UGUCAUUU | 2485 |
| 985 | CUCCUUAA G UGAUUUCU | 804 | AGAAAUCA GGAGGAAACUCC CU UCAAGGACAUCGUCCGGG UUAAGGAG | 2486 |
| 987 | CCUUAAGU G AUUUCUUC | 725 | GAAGAAAU GGAGGAAACUCC CU UCAAGGACAUCGUCCGGG ACUUAAGG | 2487 |
| 997 | UUUCUUCU G UUUUCUGU | 805 | ACAGAAAA GGAGGAAACUCC CU UCAAGGACAUCGUCCGGG AGAAGAAA | 2488 |
| 1004 | UGUUUUCU G UUUCCUUU | 806 | AAAGGAAA GGAGGAAACUCC CU UCAAGGACAUCGUCCGGG AGAAAACA | 2489 |
| 1024 | AAACAUUA G UGUUCAUA | 807 | UAUGAACA GGAGGAAACUCC CU UCAAGGACAUCGUCCGGG UAAUGUUU | 2490 |
| 1026 | ACAUUAGU G UUCAUAGC | 808 | GCUAUGAA GGAGGAAACUCC CU UCAAGGACAUCGUCCGGG ACUAAUGU | 2491 |
| 1033 | UGUUCAUA G CUUCCAAG | 809 | CUUGGAAG GGAGGAAACUCC CU UCAAGGACAUCGUCCGGG UAUGAACA | 2492 |
| 1041 | GCUUCCAA G AGACAUGC | 1055 | GCAUGUCU GGAGGAAACUCC CU UCAAGGACAUCGUCCGCG UUGGAAGC | 2493 |
| 1043 | UUCCAAGA G ACAUGCUG | 1056 | CAGCAUGU GGAGGAAACUCC CU UCAAGGACAUCGUCCGGG UCUUGGAA | 2494 |
| 1048 | AGAGACAU G CUGACUUU | 726 | AAAGUCAG GGAGGAAACUCC CU UCAAGGACAUCGUCCGGG AUGUCUCU | 2495 |
| 1051 | GACAUGCU G ACUUUCAU | 727 | AUGAAAGU GGAGGAAACUCC CU UCAAGGACAUCGUCCGGG AGCAUGUC | 2496 |
| 1065 | CAUUUCUU G AGGUACUC | 728 | GAGUACCU GGAGGAAACUCC CU UCAAGGACAUCGUCCGGG AAGAAAUG | 2497 |
| 1067 | UUUCUUGA G GUACUCUG | 1057 | CAGAGUAC GGAGGAAACUCC CU UCAAGGACAUCGUCCGGG UCAAGAAA | 2498 |
| 1068 | UUCUUGAG G UACUCUGC | 810 | GCAGAGUA GGAGGAAACUCC CU UCAAGGACAUCGUCCGGG CUCAAGAA | 2499 |
| 1075 | GGUACUCU G CACAUACG | 729 | CGUAUGUG GGAGGAAACUCC CU UCAAGGACAUCGUCCGGG AGAGUACC | 2500 |
| 1083 | GCACAUAC G CACCACAU | 730 | AUGUGGUG GGAGGAAACUCC CU UCAAGGACAUCGUCCGGG GUAUGUGC | 2501 |
| 1100 | CUCUAUCU G GCCUUUGC | 1058 | GCAAAGGC GGAGGAAACUCC CU UCAAGGACAUCGUCCGGG AGAUAGAG | 2502 |
| 1101 | UCUAUCUG G CCUUUGCA | 811 | UGCAAAGG GGAGGAAACUCC CU UCAAGGACAUCGUCCGGG CAGAUAGA | 2503 |
| 1107 | UGGCCUUU G CAUGGAGU | 731 | ACUCCAUG GGAGGAAACUCC CU UCAAGGACAUCGUCCGGG AAAGGCCA | 2504 |
| 1111 | CUUUGCAU G GAGUGACC | 1059 | GGUCACUC GGAGGAAACUCC CU UCAAGGACAUCGUCCGGG AUGCAAAG | 2505 |
| 1112 | UUUGCAUG G AGUGACCA | 1060 | UGGUCACU GGAGGAAACUCC CU UCAAGGACAUCGUCCGGG CAUGCAAA | 2506 |
| 1114 | UGCAUGGA G UGACCAUA | 812 | UAUGGUCA GGAGGAAACUCC CU UCAAGGACAUCGUCCGGG UCCAUGCA | 2507 |
| 1116 | CAUGGAGU G ACCAUAGC | 732 | GCUAUGGU GGAGGAAACUCC CU UCAAGGACAUCGUCCGGG ACUCCAUG | 2508 |
| 1123 | UGACCAUA G CUCCUUCU | 813 | AGAAGGAG GGAGGAAACUCC CU UCAAGGACAUCGUCCGGG UAUGGUCA | 2509 |
| 1142 | CUUACAUU G AAUGUAGA | 733 | UCUACAUU GGAGGAAACUCC CU UCAAGGACAUCGUCCGGG AAUGUAAG | 2510 |
| 1146 | CAUUGAAU G UAGAGAAU | 814 | AUUCUCUA GGAGGAAACUCC CU UCAAGGACAUCGUCCGGG AUUCAAUG | 2511 |
| 1149 | UGAAUGUA G AGAAUGUA | 1061 | UACAUUCU GGAGGAAACUCC CU UCAAGGACAUCGUCCGGG UACAUUCA | 2512 |
| 1151 | AAUGUAGA G AAUGUAGC | 1062 | GCUACAUU GGAGGAAACUCC CU UCAAGGACAUCGUCCGGG UCUACAUU | 2513 |
| 1155 | UAGAGAAU G UAGCCAUU | 815 | AAUGGCUA GGAGGAAACUCC CU UCAAGGACAUCGUCCGGG AUUCUCUA | 2514 |
| 1158 | AGAAUGUA G CCAUUGUA | 816 | UACAAUGG GGAGGAAACUCC CU UCAAGGACAUCGUCCGGG UACAUUCU | 2515 |
| 1164 | UAGCCAUU G UAGCAGCU | 817 | AGCUGCUA GGAGGAAACUCC CU UCAAGGACAUCGUCCGGG AAUGGCUA | 2516 |
| 1167 | CCAUUGUA G CAGCUUGU | 818 | ACAAGCUG GGAGGAAACUCC CU UCAAGGACAUCGUCCGGG UACAAUGG | 2517 |
| 1170 | UUGUAGCA G CUUGUGUU | 819 | AACACAAG GGAGGAAACUCC CU UCAAGGACAUCGUCCGGG UGCUACAA | 2518 |
| 1174 | AGCAGCUU G UGUUGUCA | 820 | UGACAACA GGAGGAAACUCC CU UCAAGGACAUCGUCCGGG AAGCUGCU | 2519 |
| 1176 | CAGCUUGU G UUGUCACG | 821 | CGUGACAA GGAGGAAACUCC CU UCAAGGACAUCGUCCGGG ACAAGCUG | 2520 |
| 1179 | CUUGUGUU G UCACGCUU | 822 | AAGCGUGA GGAGGAAACUCC CU UCAAGGACAUCGUCCGGG AACACAAG | 2521 |
| 1184 | GUUGUCAC G CUUCUUCU | 734 | AGAAGAAG GGAGGAAACUCC CU UCAAGGACAUCGUCCGGG GUGACAAC | 2522 |
| 1196 | CUUCUUUU G AGCAACUU | 735 | AAGUUGCU GGAGGAAACUCC CU UCAAGGACAUCGUCCGGG AAAAGAAG | 2523 |
| 1198 | UCUUUUGA G CAACUUUC | 823 | GAAAGUUG GGAGGAAACUCC CU UCAAGGACAUCGUCCGGG UCAAAAGA | 2524 |
| 1214 | CUUACACU G AAGAAAGG | 736 | CCUUUCUU GGAGGAAACUCC CU UCAAGGACAUCGUCCGGG AGUGUAAG | 2525 |
| 1217 | ACACUGAA G AAAGGCAG | 1063 | CUGCCUUU GGAGGAAACUCC CU UCAAGGACAUCGUCCGGG UUCAGUGU | 2526 |
| 1221 | UGAAGAAA G GCAGAAUG | 1064 | CAUUCUGC GGAGGAAACUCC CU UCAAGGACAUCGUCCGGG UUUCUUCA | 2527 |
| 1222 | GAAGAAAG G CAGAAUGA | 824 | UCAUUCUG GGAGGAAACUCC CU UCAAGGACAUCGUCCGGG CUUUCUUC | 2528 |
| 1225 | GAAAGGCA G AAUGAGUG | 1065 | CACUCAUU GGAGGAAACUCC CU UCAAGGACAUCGUCCGGG UGCCUUUC | 2529 |
| 1229 | GGCAGAAU G AGUGCUUC | 737 | GAAGCACU GGAGGAAACUCC CU UCAAGGACAUCGUCCGGG AUUCUGCC | 2530 |
| 1231 | CAGAAUGA G UGCUUCAG | 825 | CUGAAGCA GGAGGAAACUCC CU UCAAGGACAUCGUCCGGG UCAUUCUG | 2531 |
| 1233 | GAAUGAGU G CUUCAGAA | 738 | UUCUGAAG GGAGGAAACUCC CU UCAAGGACAUCGUCCGGG ACUCAUUC | 2532 |
| 1239 | GUGCUUCA G AAUGUGAU | 1066 | AUCACAUU GGAGGAAACUCC CU UCAAGGACAUCGUCCGGG UGAAGCAC | 2533 |
| 1243 | UUCAGAAU G UGAUUUCC | 826 | GGAAAUCA GGAGGAAACUCC CU UCAAGGACAUCGUCCGGG AUUCUGAA | 2534 |
| 1245 | CAGAAUGU G AUUUCCUA | 739 | UAGGAAAU GGAGGAAACUCC CU UCAAGGACAUCGUCCGGG ACAUUCUG | 2535 |
| 1261 | ACUAACCU G UUCCUUGG | 827 | CCAAGGAA GGAGGAAACUCC CU UCAAGGACAUCGUCCGGG AGGUUAGU | 2536 |
| 1268 | UGUUCCUU G GAUAGGCU | 1067 | AGCCUAUC GGAGGAAACUCC CU UCAAGGACAUCGUCCGGG AAGGAACA | 2537 |
| 1269 | GUUCCUUG G AUAGGCUU | 1068 | AAGCCUAU GGAGGAAACUCC CU UCAAGGACAUCGUCCGGG CAAGGAAC | 2538 |
| 1273 | CUUGGAUA G GCUUUUUA | 1069 | UAAAAAGC GGAGGAAACUCC CU UCAAGGACAUCGUCCGGG UAUCCAAG | 2539 |
| 1274 | UUGGAUAG G CUUUUUAG | 828 | CUAAAAAG GGAGGAAACUCC CU UCAAGGACAUCGUCCGGG CUAUCCAA | 2540 |
| 1282 | GCUUUUUA G UAUAGUAU | 829 | AUACUAUA GGAGGAAACUCC CU UCAAGGACAUCGUCCGGG UAAAAAGC | 2541 |
| 1287 | UUAGUAUA G UAUUUUUU | 830 | AAAAAAUA GGAGGAAACUCC CU UCAAGGACAUCGUCCGGG UAUACUAA | 2542 |
| 1299 | UUUUUUUU G UCAUUUUC | 831 | GAAAAUGA GGAGGAAACUCC CU UCAAGGACAUCGUCCGGG AAAAAAAA | 2543 |
| 1315 | CUCCAUCA G CAACCAGG | 832 | CCUGGUUG GGAGGAAACUCC CU UCAAGGACAUCGUCCGGG UGAUGGAG | 2544 |
| 1322 | AGCAACCA G GGAGACUG | 1070 | CAGUCUCC GGAGGAAACUCC CU UCAAGGACAUCGUCCGGG UGGUUGCU | 2545 |
| 1323 | GCAACCAG G GAGACUGC | 1071 | GCAGUCUC GGAGGAAACUCC CU UCAAGGACAUCGUCCGGG CUGGUUGC | 2546 |
| 1324 | CAACCAGG G AGACUGCA | 1072 | UGCAGUCU GGAGGAAACUCC CU UCAAGGACAUCGUCCGGG CCUGGUUG | 2547 |
| 1326 | ACCAGGGA G ACUGCACC | 1073 | GGUGCAGU GGAGGAAACUCC CU UCAAGGACAUCGUCCGGG UCCCUGGU | 2548 |
| 1330 | GGGAGACU G CACCUGAU | 740 | AUCAGGUG GGAGGAAACUCC CU UCAAGGACAUCGUCCGGG AGUCUCCC | 2549 |
| 1336 | CUGCACCU G AUGGAAAA | 741 | UUUUCCAU GGAGGAAACUCC CU UCAAGGACAUCGUCCGGG AGGUGCAG | 2550 |
| 1339 | CACCUGAU G GAAAAGAU | 1074 | AUCUUUUC GGAGGAAACUCC CU UCAAGGACAUCGUCCGGG AUCAGGUG | 2551 |
| 1340 | ACCUGAUG G AAAAGAUA | 1075 | UAUCUUUU GGAGGAAACUCC CU UCAAGGACAUCGUCCGGG CAUCAGGU | 2552 |
| 1345 | AUGGAAAA G AUAUAUGA | 1076 | UCAUAUAU GGAGGAAACUCC CU UCAAGGACAUCGUCCGGG UUUUCCAU | 2553 |
| 1352 | AGAUAUAU G ACUGCUUC | 742 | GAAGCAGU GGAGGAAACUCC CU UCAAGGACAUCGUCCGGG AUAUAUCU | 2554 |
| 1356 | AUAUGACU G CUUCAUGA | 743 | UCAUGAAG GGAGGAAACUCC CU UCAAGGACAUCGUCCGGG AGUCAUAU | 2555 |
| 1363 | UGCUUCAU G ACAUUCCU | 744 | AGGAAUGU GGAGGAAACUCC CU UCAAGGACAUCGUCCGGG AUGAAGCA | 2556 |
| 1401 | ACAUCUAC G UUUUUGGU | 833 | ACCAAAAA GGAGGAAACUCC CU UCAAGGACAUCGUCCGCG GUAGAUGU | 2557 |
| 1407 | ACGUUUUU G GUGGAGUC | 1077 | GACUCCAC GGAGGAAACUCC CU UCAAGGACAUCGUCCGGG AAAAACGU | 2558 |
| 1408 | CGUUUUUG G UGGAGUCC | 834 | GGACUCCA GGAGGAAACUCC CU UCAAGGACAUCGUCCGGG CAAAAACG | 2559 |
| 1410 | UUUUUGGU G GAGUCCCU | 1078 | AGGGACUC GGAGGAAACUCC CU UCAAGGACAUCGUCCGGG ACCAAAAA | 2560 |
| 1411 | UUUUGGUG G AGUCCCUU | 1079 | AAGGGACU GGAGGAAACUCC CU UCAAGGACAUCGUCCGGG CACCAAAA | 2561 |
| 1413 | UUGGUGGA G UCCCUUUU | 835 | AAAAGGGA GGAGGAAACUCC CU UCAAGGACAUCGUCCGGG UCCACCAA | 2562 |
| 1422 | UCCCUUUU G CAUCAUUG | 745 | CAAUGAUG GGAGGAAACUCC CU UCAAGGACAUCGUCCGGG AAAAGGGA | 2563 |
| 1430 | GCAUCAUU G UUUUAAGG | 836 | CCUUAAAA GGAGGAAACUCC CU UCAAGGACAUCGUCCGGG AAUGAUGC | 2564 |
| 1437 | UGUUUUAA G GAUGAUAA | 1080 | UUAUCAUC GGAGGAAACUCC CU UCAAGGACAUCGUCCGGG UUAAAACA | 2565 |
| 1438 | GUUUUAAG G AUGAUAAA | 1081 | UUUAUCAU GGAGGAAACUCC CU UCAAGGACAUCGUCCGGG CUUAAAAC | 2566 |
| 1441 | UUAAGGAU G AUAAAAAA | 746 | UUUUUUAU GGAGGAAACUCC CU UCAAGGACAUCGUCCGGG AUCCUUAA | 2567 |
| 1463 | AACAACUA G GGACAAUA | 1082 | UAUUGUCC GGAGGAAACUCC CU UCAAGGACAUCGUCCGGG UAGUUGUU | 2568 |
| 1464 | ACAACUAG G GACAAUAC | 1083 | GUAUUGUC GGAGGAAACUCC CU UCAAGGACAUCGUCCGGG CUAGUUGU | 2569 |
| 1465 | CAACUAGG G ACAAUACA | 1084 | UGUAUUGU GGAGGAAACUCC CU UCAAGGACAUCGUCCGGG CCUAGUGG | 2570 |
| 1474 | ACAAUACA G AACCCAUU | 1085 | AAUGGGUU GGAGGAAACUCC CU UCAAGGACAUCGUCCGGG UGUAUGGU | 2571 |
| 1500 | UUUCUACA G GGCUGACA | 1086 | UGUCAGCC GGAGGAAACUCC CU UCAAGGACAUCGUCCGGG UGUAGAAA | 2572 |
| 1501 | UUCUACAG G GCUGACAU | 1087 | AUGUCAGC GGAGGAAACUCC CU UCAAGGACAUCGUCCGGG CUGUAGAA | 2573 |
| 1502 | UCUACAGG G CUGACAUU | 837 | AAUGUCAG GGAGGAAACUCC CU UCAAGGACAUCGUCCGGG CCUGUAGA | 2574 |
| 1505 | ACAGGGCU G ACAUUGUG | 747 | CACAAUGU GGAGGAAACUCC CU UCAAGGACAUCGUCCGGG AGCCCUGU | 2575 |
| 1511 | CUGACAUU G UGGCACAU | 838 | AUGUGCCA GGAGGAAACUCC CU UCAAGGACAUCGUCCGGG AAUGUCAG | 2576 |
| 1513 | GACAUUGU G GCACAUUC | 1088 | GAAUGUGC GGAGGAAACUCC CU UCAAGGACAUCGUCCGGG ACAAUGUC | 2577 |
| 1514 | ACAUUGUG G CACAUUCU | 839 | AGAAUGUG GGAGGAAACUCC CU UCAAGGACAUCGUCCGGG CACAAUGU | 2578 |
| 1525 | CAUUCUUA G AGUUACCA | 1089 | UGGUAACU GGAGGAAACUCC CU UCAAGGACAUCGUCCGGG UAAGAAUG | 2579 |
| 1527 | UUCUUAGA G UUACCACA | 840 | UGUGGUAA GGAGGAAACUCC CU UCAAGGACAUCGUCCGGG UCUAAGAA | 2580 |
| 1542 | CACCCCAU G AGGGAAGC | 748 | GCUUCCCU GGAGGAAACUCC CU UCAAGGACAUCGUCCGGG AUGGGGUG | 2581 |
| 1544 | CCCCAUGA G GGAAGCUC | 1090 | GAGCUUCC GGAGGAAACUCC CU UCAAGGACAUCGUCCGGG UCAUGGGG | 2582 |
| 1545 | CCCAUGAG G GAAGCUCU | 1091 | AGAGCUUC GGAGGAAACUCC CU UCAAGGACAUCGUCCGGG CUCAUGGG | 2583 |
| 1546 | CCAUGAGG G AAGCUCUA | 1092 | UAGAGCUU GGAGGAAACUCC CU UCAAGGACAUCGUCCGGG CCUCAUGG | 2584 |
| 1549 | UGAGGGAA G CUCUAAAU | 841 | AUUUAGAG GGAGGAAACUCC CU UCAAGGACAUCGUCCGGG UUCCCUCA | 2585 |
| 1559 | UCUAAAUA G CCAACACC | 842 | GGUGUUGG GGAGGAAACUCC CU UCAAGGACAUCGUCCGGG UAUUUAGA | 2586 |
| 1573 | ACCCAUCU G UUUUUUGU | 843 | ACAAAAAA GGAGGAAACUCC CU UCAAGGACAUCGUCCGGG AGAUGGGU | 2587 |
| 1580 | UGUUUUUU G UAAAAACA | 844 | UGUUUUUA GGAGGAAACUCC CU UCAAGGACAUCGUCCGGG AAAAAACA | 2588 |
| 1589 | UAAAAACA G CAUAGCUU | 845 | AAGCUAUG GAGGGAAACUCC CU UCAAGGACAUCGUCCGGG UGUUUUUA | 2589 |

Input Sequence = HSCD20A. Cut Site = C/.
Stem Length = 8. Core Sequence = GGAGGAAACUCC CU UCAAGGACAUCGUCCGGG
HSCD20A (Human mRNA for CD20 receptor (S7) ; 1597 bp)

What is claimed is:

1. A nucleic acid molecule which down regulates expression of a CD20 gene.

2. The nucleic acid of claim 1, wherein said nucleic acid molecule is used to treat conditions selected from the group consisting of lymphoma, leukemia, arthropathy, B-cell lymphoma, low-grade or follicular non-Hodgkin's lymphoma (NHL), bulky low-grade or follicular NHL, lypmphocytic leukemia, HIV associated NHL, mantle-cell lymphoma (MCL), immunocytoma (IMC), small B-cell lymphocytic lymphoma, immune thrombocytopenia, and inflammatory arthropathy.

3. The nucleic acid molecule of claim 1, wherein said nucleic acid molecule is an enzymatic nucleic acid molecule having one or more binding arms.

4. The nucleic acid of claim 3, wherein said binding arm comprises a sequence complementary to a sequence selected from the group consisting of SEQ ID NOs. 1-1092.

5. The nucleic acid molecule of claim 3, wherein said enzymatic nucleic acid molecule comprises a sequence selected from the group consisting of SEQ ID NOs. 1093-2589.

6. The nucleic acid molecule of claim 1, wherein said nucleic acid molecule is an antisense nucleic acid molecule.

7. The nucleic acid molecule of claim 6, wherein said antisense nucleic acid molecule comprises a sequence selected from the group consisting of SEQ ID NOs. 1-1092.

8. The nucleic acid molecule of claim 3, wherein said enzymatic nucleic acid molecule is in a hammerhead (HH) motif.

9. The nucleic acid molecule of claim 3, wherein said enzymatic nucleic acid molecule is in a hairpin, hepatitis Delta virus, group I intron, VS nucleic acid, amberzyme, zinzyme or RNAse P nucleic acid motif.

10. The nucleic acid molecule of claim 3, wherein said enzymatic nucleic acid molecule is in an Inozyme motif.

11. The nucleic acid molecule of claim 3, wherein said enzymatic nucleic acid molecule is in a G-cleaver motif.

12. The nucleic acid molecule of claim 3, wherein said enzymatic nucleic acid molecule is a DNAzyme.

13. The nucleic acid molecule of claim 3, wherein said enzymatic nucleic acid molecule comprises between 12 and 100 bases complementary to the RNA of CD20 gene.

14. The nucleic acid of claim 3, wherein said enzymatic nucleic acid molecule comprises between 14 and 24 bases complementary to the RNA of CD20 gene.

15. The nucleic acid molecule of claim 1, wherein said nucleic acid is chemically synthesized.

16. The nucleic acid molecule of claim 1, wherein said nucleic acid comprises at least one 2′-sugar modification.

17. The nucleic acid molecule of claim 1, wherein said nucleic acid comprises at least one nucleic acid base modification.

18. The nucleic acid molecule of claim 1, wherein said nucleic acid comprises at least one phosphate backbone modification.

19. A mammalian cell comprising the nucleic acid molecule of claim 1.

20. The mammalian cell of claim 19, wherein said mammalian cell is a human cell.

21. A method of reducing CD20 activity in a cell, comprising the step of contacting said cell with the nucleic acid molecule of claim 1, under conditions suitable for said reduction of CD20 activity.

22. A method of treatment of a patient having a condition associated with the level of CD20, comprising contacting cells of said patient with the nucleic acid molecule of claim 1, under conditions suitable for said treatment.

23. The method of claim 22 further comprising the use of one or more therapies under conditions suitable for said treatment.

24. A method of cleaving RNA of CD20 gene comprising contacting the nucleic acid molecule of claim 3, with said RNA under conditions suitable for the cleavage of said RNA.

25. The method of claim 24, wherein said cleavage is carried out in the presence of a divalent cation.

26. The method of claim 25, wherein said divalent cation is Mg²⁺.

27. The nucleic acid molecule of claim 1, wherein said nucleic acid comprises a cap structure, wherein the cap structure is at the 5′-end or 3′-end or both the 5′-end and the 3′-end.

28. The enzymatic nucleic acid molecule of claim 8, wherein said hammerhead motif comprises a sequence selected from the group consisting of SEQ ID NOs. 1-348.

29. The enzymatic nucleic acid molecule of claim 10, wherein said NCH motif comprises a sequence selected from the group consisting of SEQ JD NOs. 349-684.

30. The enzymatic nucleic acid molecule of claim 11, wherein said G-cleaver motif comprises a sequence selected from the group consisting of SEQ ID NOs. 685-748.

31. An expression vector comprising a nucleic acid sequence encoding at least one nucleic acid molecule of claim 1, in a manner which allows expression of the nucleic acid molecule.

32. A mammalian cell comprising the an expression vector of claim 34.

33. The mammalian cell of claim 35, wherein said mammalian cell is a human cell.

34. The expression vector of claim 34, wherein said nucleic acid molecule is an enzymatic nucleic acid molecule

35. The expression vector of claim 34, wherein said expression vector further comprises a sequence for an antisense nucleic acid molecule complementary to the RNA of CD20 gene.

36. The expression vector of claim 34, wherein said expression vector comprises a sequence encoding at least two of said nucleic acid molecules, which may be the same or different.

37. The expression vector of claim 39, wherein said expression vector further comprises a sequence encoding an antisense nucleic acid molecule complementary to the RNA of CD20 gene.

38. The expression vector of claim 39, wherein said expression vector further comprises a sequence encoding an enzymatic nucleic acid molecule complementary to the RNA of CD20 gene.

39. A method for the treatment of lymphoma, comprising the step of administering to a patient the nucleic acid molecule of claim 1 under conditions suitable for said treatment.

40. A method for the treatment of leukemia, comprising the step of administering to a patient the nucleic acid molecule of claim 1 under conditions suitable for said treatment.

41. An enzymatic nucleic acid molecule which cleaves RNA derived from CD20 gene.

42. The enzymatic nucleic acid molecule of claim 44, wherein said enzymatic nucleic acid molecule is selected from the group consisting of Hammerhead, Hairpin, Inozyme, G-cleaver, DNAzyme, Amberzyme and Zinzyme.

43. The method of claim 42 or claim 43, wherein said method further comprises administering to said patient the nucleic acid molecule of claim 1 in conjunction with one or more other therapies.

44. The method of claim 46, wherein the other therapies are selected from the group consisting of radiation, chemotherapy, and cyclosporin treatment.

45. The nucleic acid molecule of claim 6, wherein said nucleic acid molecule comprises at least five ribose residue; at least ten 2′-O-methyl modifications, and a 3′-end modification.

46. The nucleic acid molecule of claim 48, wherein said nucleic acid molecule further comprises a phosphorothioate core with both 3′ and 5′-end modifications.

47. The nucleic acid molecule of claim 48 or claim 49, wherein said 3′ and/or 5′-end modification is 3′-3′ inverted abasic moiety.

48. The nucleic acid molecule of claim 3, wherein said nucleic acid molecule comprises at least five ribose residue, at least ten 2′-O-methyl modifications, and a 3′-end modification.

49. The nucleic acid molecule of claim 51, wherein said nucleic acid molecule further comprises phosphorothioate linkages on at least three of the 5′ terminal nucleotides.

50. The nucleic acid molecule of claim 51, wherein said 3′-end modification is a 3′-3′ inverted abasic moiety.

51. The enzymatic nucleic acid molecule of claim 12, wherein said DNAzyme comprises at least ten 2′-O-methyl modifications and a 3′-end modification.

52. The enzymatic nucleic acid molecule of claim 54, wherein said DNAzyme further comprises phosphorothioate linkages on at least three of the 5′ terminal nucleotides.

53. The enzymatic nucleic acid molecule of claim 54, wherein said 3′-end modification is 3′-3′ inverted abasic moiety.
